(19) United States
(12) Patent Application Publication
(10) Pub. No.: US 2026/0000626 A1
(43) Pub. Date: Jan. 1, 2026

(54) POLYMERIC NANOPARTICLES FOR LONG ACTING DELIVERY OF A PEPTIDE AND METHODS OF MAKING AND USING THEREOF

(71) Applicant: Brown University, Providence, RI (US)

(72) Inventors: Edith Mathiowitz, Brookline, MA (US); Cameron Baptista, Phillipsburg, NJ (US)

(21) Appl. No.: 18/880,822

(22) PCT Filed: Jul. 7, 2023
(86) PCT No.: PCT/US2023/069771

§ 371 Date: Jan. 2, 2025

Related U.S. Application Data
(60) Provisional application No. 63/359,293, filed on Jul. 8, 2022.
(60) Provisional application No. 63/375,110, filed on Sep. 9, 2022.

(51) Int. Cl. A61K 9/51; A61K 38/26

(52) U.S. Cl. CPC A61K 9/5153; A61K 9/5192; A61K 38/26

(57) ABSTRACT

Disclosed herein are polymeric nanoparticles containing peptides, which provide low burst release and sustained, delivery of the peptides, and pharmaceutical compositions thereof. The polymeric nanoparticles contain a peptide encapsulated or dispersed therein. The nanoparticles can provide sustained release of the peptide, for example, less than 20% of the peptide is released initially (at time 0 hour) following placement into a phosphate buffered saline at pH 7.4 at 37° C. and room pressure. Methods for micronizing a peptide and for preparing polymeric nanoparticles containing solid, micronized peptides are also disclosed. The preparation methods use miscible solvent and non-solvent pairs in phase inversion nanoencapsulation processes. The Gibbs energy of mixing (ΔG_{Mix}) between the solvent and non-solvent can be tailored to achieve desired particle size, encapsulation efficiency, and release profile.

### CROSS-REFERENCE TO RELATED APPLICATIONS

This application claims the benefit of and priority to U.S. Application No. 63/359,293 filed Jul. 8, 2022, and U.S. Application No. 63/375,110 filed Sep. 9, 2022, the disclosures of which are incorporated herein by reference.

### REFERENCE TO THE SEQUENCE LISTING

The Sequence Listing submitted as a text file named “BU_3253_PCT_ST26.xml” created on Jul. 7, 2023, and having a size of 12,770 bytes is hereby incorporated by reference pursuant to 37 C.F.R. § 1.834 (c) (1).

### FIELD OF THE INVENTION

This invention is generally in the field of polymeric nanoparticles and methods of making and using hereof.

### BACKGROUND OF THE INVENTION

Generally, one of the major challenges in the field of peptide and protein therapeutics is their delivery to the site of action. Due to their high molecular weight and susceptibility to degradation by both enzymes and extreme pH values, peptides and proteins usually display poor absorption across epithelial membranes and exhibit low oral bioavailability. For this reason, peptide therapeutics such as GLP-1 RAs are typically administered by subcutaneous injection. In some cases, even upon injection, peptides can be rapidly metabolized and cleared from circulation and therefore require several injections to be administered daily. Thus, from the perspective of both the pharmaceutical industry and patients alike, the ability to reduce the frequency of injections or convert an approved injectable peptide or protein into a non-injected formulation would represent a major advance in treatment.

There are many unique challenges in the formulation of peptide and protein nanoparticle delivery systems that must be addressed to achieve consistent clinical performance. To start, a key barrier to clinical success is often the ability to achieve a tailored sustained release profile of the drug cargo from the nanoparticle delivery system. Compounding this challenge, the size, surface charge, and shape of the nanoparticles must also be tailored as these factors have a role in the biodistribution and fate of the drug in vivo. To add, even in instances where successful therapeutic performance is achieved, formulations often face technical challenges associated with drug stability, drug loading efficiency, scale up feasibility, batch to batch consistency, and economic viability. For example, peptide and protein instability associated with process, storage, and delivery remains a major challenge in the formulation of biologic therapeutics.

There have been a number of studies which report the encapsulation of GLP-1 RAs in PLGA or polylactic acid (PLA) nanoparticles. However, AstraZeneca's Bydureon® is the only PLGA particle formulation to reach the market for the delivery of a GLP-1 RA. Bydureon® is a relatively large particle formulation (particle size ˜50 μm) and is currently limited to a once weekly subcutaneous injection. Currently, the most common methods for producing polymeric nanoparticles require an initial step of emulsifying a polymer solvent in an aqueous non-solvent. This emulsification step poses many challenges for the formulations containing peptide and protein drugs. Both the high sheer rate and oil-water interfaces formed during emulsification are prone to destroying the secondary and tertiary structures of the API. These emulsion processes may also be riddled with low encapsulation efficiencies, due to the use of an aqueous phase in which the drug is soluble. In addition, the emulsion droplet size is often a limiting factor on the ultimate size of the nanoparticles produced. A method termed phase inversion nanoencapsulation (PIN) is designed to combat these issues. The PIN process is based on the mechanism of precipitation by phase inversion and thus utilizes solvent and non-solvent pairs that are completely miscible, avoiding these key issues associated with emulsification. In the PIN process, nanoparticles spontaneously precipitate after the immersion of a solubilized polymer solution in a non-solvent. Upon addition, the mechanism of polymer phase inversion is believed to occur in three key steps: supersaturation, nucleation, and growth.

However, there exists a need for improved methods of micronizing peptides. There also exists a need for improved method for forming polymeric nanoparticles, particularly nanoparticles that encapsulate peptides or have peptides dispersed therein. There also exists a need for compositions that provide low or minimal burst release of an active agent, such as a peptide, and also provide prolonged release of the active agent.

Therefore, it is an object of the invention to provide improved methods of micronizing peptides.

It is a further objection of the invention to provide methods for forming polymeric nanoparticles, particularly nanoparticles that encapsulate peptides or have peptides dispersed therein.

It is another object of the invention to provide compositions that provide low or minimal burst release of an active agent, such as a peptide, and also provide prolonged release of the active agent.

### SUMMARY OF THE INVENTION

Disclosed herein are polymeric nanoparticles containing peptides, which provide low burst release and sustained, delivery of the peptides, and pharmaceutical compositions thereof. The polymeric nanoparticles contain a peptide encapsulated or dispersed therein. The nanoparticles can provide sustained release of the peptide, for example, less than 20% of the peptide is released initially (at time 0 hour) following placement into a phosphate buffered saline at pH 7.4 at 37° C. and room pressure. The nanoparticles described herein can be formulated into pharmaceutical composition or formulations, such as for oral administration, intraperitoneal administration, nasal administration, and/or intravenous administration. For example, the nanoparticles can contain a glucagon-like peptide-1 receptor agonist (GLP-1 RA). These nanoparticles can be formulated into pharmaceutical compositions or formulations for administration to patients in need of treatment for type 2 diabetes (T2D).

Methods for micronizing a peptide are also disclosed. Generally, the method includes (i) dissolving the peptide in an effective amount of a peptide solvent, wherein the peptide and the peptide solvent to form a peptide solution, and (ii) introducing the peptide solution into a peptide non-solvent. Typically, the peptide solvent and the peptide non-solvent are miscible, i.e. the Gibbs energy of mixing (ΔGₘᵢₓ/RT) for the peptide solvent and the peptide non-solvent is negative. For example, the Gibbs energy of mixing (ΔGₘᵢₓ/RT) for the peptide solvent and the peptide non-solvent is less than or equal to about −0.6.

Methods for preparing polymeric nanoparticles containing solid, micronized peptides are also disclosed. The solid, micronized peptides can be encapsulated or dispersed in the nanoparticles. Generally, the method includes: (a) dissolving a polymer in a first suspension containing the solid micronized peptide and a polymer solvent to form a second suspension, and (b) introducing the second suspension into a polymer non-solvent to spontaneously form the nanoparticle. In step (a), the polymer and the solid micronized peptide are in the polymer solvent, and the polymer is soluble in the polymer solvent. In step (b), the polymer non-solvent is also a non-solvent for the peptide. The polymer solvent and the polymer non-solvent are miscible, i.e. the Gibbs energy of mixing (ΔGₘᵢₓ/RT) for the polymer solvent and the polymer non-solvent is negative. For example, the Gibbs energy of mixing (ΔGₘᵢₓ/RT) for the polymer solvent and the polymer non-solvent is less than or equal to about −0.6.

The methods for preparing the polymeric nanoparticles encapsulating peptides described herein can use any suitable solvents and non-solvents, as long as the ΔG_{Mix} of the solvent and non-solvent pairs has a desired value, selected based on the desired size, encapsulation efficiency, and/or release profile. For example, when the desired release profile of the formed nanoparticles is sustained release over a long period of time, such as a lag time period ≥200 hours, without burst release (i.e. ≤20% of theoretical loading at time 0 hr), then the ΔG_{Mix} of the solvent and non-solvent pairs is more negative, such as equal to or more negative than −0.6 (i.e. less than or equal to about −0.6). When the desired release profile of the formed nanoparticles is rapid release over a short period of time, such as a lag time period ≤24 hours, with burst release (i.e. >20% of theoretical loading at time 0 hr), then the ΔG_{Mix} of the solvent and non-solvent pairs is less negative, such as less negative than −0.6 (i.e., more than −0.6, such as −0.2).

In some embodiments, the peptide can be micronized using the method described above for micronizing peptides and then encapsulated in polymeric nanoparticles using the method described herein. In these embodiments, the non-solvent for the peptide (i.e., peptide non-solvent) is optionally selected such that it is also the solvent for the polymer (i.e., polymer solvent). Thus, in some embodiments, the peptide non-solvent is the same as the polymer solvent.

### BRIEF DESCRIPTION OF THE DRAWINGS

FIGS. 1A-1I show representative SEM images of micronized GLP-1 RA produced from phase inversion with different solvent and non-solvent pairs: water and tert-butanol (FIG. 1A), methanol and tert-butanol (FIG. 1B), methanol and acetone (FIG. 1C), methanol and acetonitrile (FIG. 1D), methanol and DCM (FIG. 1E), methanol and chloroform (FIG. 1F), methanol and ethyl acetate (FIG. 1G), methanol and THF (FIG. 1H), and methanol and 2-propanol (FIG. 1I).

FIG. 2 is a graph showing the relationship between the average size (i.e. hydrodynamic diameter) (nm) of micronized GLP-1 RA (y-axis) and the Gibbs free energy of mixing between the solvent and non-solvent pair in the micronization process (x-axis).

FIG. 3 shows a representative SEM image of the GLP-1 RA peptide as received from Sanofi S.A (i.e. prior to micronization).

FIGS. 4A-4B show representative images of micronized GLP-1 RA particles formed using 1 mg/mL GLP-1 RA in water (FIG. 4A) and 20 mg/mL GLP-1 RA in water (FIG. 4B), shown at the same magnification level to illustrate the effect of the GLP-1 RA concentration on micronization particle size.

FIG. 5 shows the FTIR spectrum of the GLP-1 RA peptide with labeled Amide, C—H and P—O bond excitements. The expanded view illustrates the deconvolution of the Amide I band based on secondary structure components.

FIGS. 6A-6B are graphs showing the peak deconvolution comparison of the FTIR spectrum Amide I band observed for micronized GLP-1 RA produced from GLP-1 RA as received and a representative organic solvent and non-solvent pair: GLP-1 RA as received (FIG. 6A) and methanol and TBA (FIG. 6B).

FIGS. 7A-7J are representative HPLC Chromatograms of GLP-1RA elution time (minutes) after micronization with different solvent and non-solvent combinations: standard GLP-1RA (FIG. 7A), methanol only (FIG. 7B), methanol and THF (FIG. 7C), methanol and propanol (FIG. 7D), methanol and ACN (FIG. 7E), methanol and ethyl acetate (FIG. 7F), methanol and TBA (FIG. 7G), methanol and DCM (FIG. 7H), methanol and chloroform (FIG. 7I), methanol and acetone (FIG. 7J). The shape of the HPLC curve and retention time was used to gain insight into peptide stability.

FIGS. 8A-8U are SEM images of exemplary polymeric (PLGA) nanoparticles produced from phase inversion with varying solvent and non-solvents: dichloromethane and ethanol (FIG. 8A), dichloromethane and 2-propanol (FIG. 8B), dichloromethane and tert-butanol (FIG. 8C), dichloromethane and heptane (FIG. 8D), chloroform and ethanol (FIG. 8E), chloroform and 2-propanol (FIG. 8F), chloroform and tert-butanol (FIG. 8G), chloroform and heptane (FIG. 8H), ethyl acetate and ethanol (FIG. 8I), ethyl acetate and 2-propanol (FIG. 8J), ethyl acetate and tert-butanol (FIG. 8K), ethyl acetate and heptane (FIG. 8L), acetonitrile and ethanol (FIG. 8M), acetonitrile and 2-propanol (FIG. 8N), acetonitrile and tert-butanol (FIG. 8O), acetonitrile and water (FIG. 8P), acetone and ethanol (FIG. 8Q), acetone and 2-propanol (FIG. 8R), acetone and tert-butanol (FIG. 8S), acetone and heptane (FIG. 8T), and acetone and water (FIG. 8U). The images were taken using magnification 8000× (FIGS. 8A-8O, 8Q-8T), 6500× (FIG. 8P), or 10000× (FIG. 8U).

FIG. 9 is a graph showing the relationship between PLGA nanoparticle hydrodynamic diameter size (y-axis) and the Gibbs energy of mixing between solvents in the phase inversion process (x-axis).

FIGS. 10A-10F are graphs showing the GLP-1 RA release curves from all nanoparticle formulations produced. Each row presents a different type of PLGA used: PLGA (75:25) 4-15 kDa (FIGS. 10A and 10D), PLGA (50:50) 2.3 kDa (FIGS. 10B and 10E), and PLGA (50:50) 7-17 kDa (FIGS. 10C and 10F). Each column presents a different PLGA/GLP-1 RA non-solvent used: 2-Propanol (FIGS. 10A-10C) and Heptane (FIGS. 10D-10F). Within each individual graph, the corresponding curves represent formulations produced with different PLGA solvents/GLP-1 RA non-solvents (i.e., dichloromethane, chloroform, tetrahydrofuran, ethyl acetate, acetone, and acetonitrile)

FIGS. 11A-11B are graphs for showing the GLP-1 RA release curves from PLGA nanoparticles produced with dichloromethane and chloroform (FIG. 11A) or ethyl acetate and tetrahydrofuran as a polymer solvent (FIG. 11B).

FIG. 12A-12B are graphs showing the relationship between Gibbs energy of mixing between the solvent and non-solvent used in the PIN process and the burst release of GLP-1 RA at the 0 hr time point (FIG. 12A) or the diffusion release of GLP-1 RA after 24 hours (FIG. 12B).

FIGS. 13A-13B are graphs showing the relationship between the PLGA and solvent X₁₂ and the corresponding burst release of GLP-1 RA at the 0 hr time point (FIG. 13A) or diffusion release of GLP-1 RA after 24 hours (FIG. 13B).

FIG. 14 is a graph showing the representative release curves produced with dichloromethane as a solvent and 2-propanol as a non-solvent, demonstrating the variation in release behavior between different types of PLGAs.

FIGS. 15A-15B are graphs showing the in-vivo GLP-1RA bioactivity after encapsulation and release from PLGA nanoparticles: blood glucose concentration over 8 hours (FIG. 15A) and plasma insulin concentration at 0.5 and 1 hours after administration (FIG. 15B).

### DETAILED DESCRIPTION OF THE INVENTION

### I. Compositions

Polymeric nanoparticles containing peptides and pharmaceutical compositions thereof are disclosed herein. The polymeric nanoparticles can provide low burst release and sustained, delivery of the peptides. The term “low burst release” refers to release of 0-20% of the theoretical loading at time 0, such as when the nanoparticles are placed in PBS (pH 7.4) at 37° C. and room pressure (i.e., 1 atm), without mixing.

These polymeric nanoparticles can be formulated into pharmaceutical compositions or formulations suitable for a variety of administrations, such as for oral administration and/or intravenous administration. An exemplary polymeric nanoparticle is PLGA nanoparticle containing glucagon-like peptide-1 receptor agonist (“GLP-1 RA”).

### A. Polymeric Nanoparticles

The disclosed polymeric nanoparticle contains one or more peptides encapsulated or dispersed therein, which provides sustained release of the peptide(s). For example, the nanoparticle provides sustained release of the peptide with less than 20% of the peptide released initially (at time 0 hour) following placement into a phosphate buffered saline at pH 7.4 at 37° C. and room pressure. In some embodiments, the nanoparticles can release less than 50% of the peptide at 24 hours following placement into the phosphate buffered saline. In some embodiments, the nanoparticles can release less than 50% of the peptide at 200 hours following placement into the phosphate buffered saline. In some embodiments, the nanoparticles can release ≥50% of the peptide at or after 400 hours following placement into the phosphate buffered saline.

The polymeric nanoparticles can have a number average size less than 1 micron, such as in a range from about 10 nm to about 1 micron, from about 10 nm to about 500 nm, from about 10 nm to about 400 nm, from about 10 nm to about 300 nm, from about 50 nm to about 500 nm, from about 50 nm to about 400 nm, from about 50 nm to about 300 nm, from about 50 nm to about 250 nm, from about 100 nm to about 500 nm, from about 100 nm to about 400 nm, or from about 100 nm to about 300 nm. In some embodiments, the polymeric nanoparticles can have a number average size of about 300 nm or less, such as in a range from about 10 nm to about 300 nm, from about 50 nm to about 300 nm, or from about 100 nm to about 300 nm.

The size and/or release profiles of the nanoparticles can be tuned, such as by selecting Gibbs energy of mixing (ΔGₘᵢₓ/RT) for the polymer solvent and the polymer non-solvent forming the nanoparticles (described in details below).

### 1. Peptides

Any suitable peptide can be micronized and/or encapsulated in the nanoparticles described herein. Generally, the peptide has a molecular weight of 6,000 Da or less. Exemplary peptides include, but are not limited to, glucagon, pramlintide, insulin, leuprolide, an luteinizing-hormone-releasing hormone (LHRH) agonist, parathyroid hormone (PTH) or its pharmaceutically active sub-units, amylin, botulinum toxin, hematide, an amyloid peptide, cholecystikinin, gastric inhibitory peptide, an insulin-like growth factor, growth hormone releasing factor, anti-microbial factor, glatiramer, glucagon-like peptide-1 (GLP-1), a GLP-1 agonist, e.g., exenatide, interferons, insulin, insulin analogs, c-peptide, amylin, analogues thereof, and mixtures thereof.

For example, the peptide can be a Glucagon-like peptide-1 (GLP-1) or a truncated biologically active portion thereof or an analogue thereof.

a. Glucagon-Like Peptide-1

Glucagon-like peptide-1 (GLP-1), a member of the glucagon peptide family, is a 30 amino acid long peptide hormone deriving from the tissue-specific posttranslational processing of the proglucagon gene.

Human GLP-1 (1-37) has the amino acid sequence:

(SEQ ID NO: 1)
HDEFERHAEGTFTSDVSSYLEGQAAKEFIAWLVKGRG.

The initial product GLP-1 (1-37) is susceptible to amidation and proteolytic cleavage, which gives rise to the two truncated and equipotent biologically active forms, GLP-1 (7-36) amide and GLP-1 (7-37).

Human GLP-1 (7-37) has the amino acid sequence:

(SEQ ID NO: 2)
HAEGTFTSDVSSYLEGQAAKEFIAWLVKGRG.

Human GLP-1 (7-36) has the amino acid sequence:

(SEQ ID NO: 3)
HAEGTFTSDVSSYLEGQAAKEFIAWLVKGR

Active GLP-1 contains two a-helices from amino acid position 13-20 and 24-35 (of SEQ ID NO:1) separated by a linker region.

DPP-IV cleaves the peptide bond in Ala8-Glu9 (of SEQ ID NO:1), and the resulting metabolite GLP-1(9-36)-NH₂ is found to have 100-fold lower binding affinity compared to the intact peptide (Manadhar and Ahn, J. Med. Chem. 2015, 58, 1020-1037). The metabolite also exhibits negligible agonistic activity (>10000-fold decrease).

b. Glucagon-Like Peptide-1 Analogues

Modifying the two sites in the GLP-1 molecule susceptible to cleavage: the position 8 alanine and the position 34 lysine, can help prolong the half-life of GLP-1. These, and other chemical modifications, help in creating compounds known as GLP-1 receptor agonists, which have a longer half-life, and can be used for therapeutic purposes.

Suitable GLP-1 analogues include, for example, exenatide (BYETTA®, BYDUREON®), liraglutide (VICTOZA®, SAXENDA®), lixisenatide (LYXUMIA®, ADLYXIN®), albiglutide (TANZEUM™), dulaglutide (TRULICITY®), semaglutide (OZEMPIC®), and taspoglutide.

i. Exenatide

Exenatide, a functional analogue of GLP-1, is a synthetic version of exendin-4, a hormone found in the saliva of the Gila monster. Exenatide has the amino acid sequence:

(SEQ ID NO: 4)
HGEGTFTSDLSKQMEEEAVRLFIEWLKNGGPSSGAPPPS.

BYETTA® is an immediate-release exenatide formulated for subcutaneous (SC) injection. \

ii. Liraglutide

Liraglutide is a long-acting, fatty acylated GLP-1 analogue with prolonged action and half-life of 11-15 hours. The improved properties of liraglutide are credited to the attachment of the fatty acid palmitic acid to GLP-1 that reversibly binds to albumin and protects it from degradation and elimination and facilitates slow and consistent release. Liraglutide has the amino acid sequence HAEGTFTSDVSSYLEGQAAKEFIAWLVRGRG (SEQ ID NO:5), and has a C-16 fatty acid (palmitic acid) attached with a glutamic acid spacer on the lysine residue at position 26 of the peptide precursor (bold/italics in SEQ ID NO:5). Liraglutide is 97% homologous to native human GLP-1 with a substituted arginine for lysine at position 34.

VICTOZA® and SAXENDA® are liraglutide formulations for subcutaneous injection. \

iii. Lixisenatide

Lixisenatide is “des-38-proline-exendin-4 (Heloderma suspectum)-(1-39)-peptidylpenta-L-lysyl-L-lysinamide,” meaning it is derived from the first 39 amino acids in the sequence of the peptide exendin-4, omitting proline at position 38 and adding six lysine residues. The amino acid sequence of lixisenatide is

(SEQ ID NO: 6)
HGEGTFTSDLSKQMEEEAVRLFIEWLKNGGPSSGAPPSKKKKKK.

ADLYXIN® and LYXUMIA® are lixisenatide formulations for subcutaneous injection.

iv. Albiglutide

Albiglutide is a dipeptidyl peptidase-4-resistant GLP-1 dimer fused to human albumin. The two GLP-1-likes domains have a single amino acid substitution relative to GLP-1(7-36). The amino acid sequence for albiglutide is:

(SEQ ID NO: 7)
HGEGTFTSDVSSYLEGQAAKEFIAWLVKGRHGEGTFTSDVSSYLEGQAA
KEFIAWLVKGRDAHKSEVAHRFKDLGEENFKALVLIAFAQYLQQCPFED
HVKLVNEVTEFAKTCVADESAENCDKSLHTLFGDKLCTVATLRETYGEM
ADCCAKQEPERNECFLQHKDDNPNLPRLVRPEVDVMCTAFHDNEETFLK
KYLYEIARRHPYFYAPELLFFAKRYKAAFTECCQAADKAACLLPKLDEL
RDEGKASSAKQRLKCASLQKFGERAFKAWAVARLSQRFPKAEFAEVSKL
VTDLTKVHTECCHGDLLECADDRADLAKYICENQDSISSKLKECCEKPL
LEKSHCIAEVENDEMPADLPSLAADFVESKDVCKNYAEAKDVFLGMFLY
EYARRHPDYSVVLLLRLAKTYETTLEKCCAAADPHECYAKVFDEFKPLV
EEPQNLIKQNCELFEQLGEYKFQNALLVRYTKKVPQVSTPTLVEVSRNL
GKVGSKCCKHPEAKRMPCAEDYLSVVLNQLCVLHEKTPVSDRVTKCCTE
SLVNRRPCFSALEVDETYVPKEFNAETFTFHADICTLSEKERQIKKQTA
LVELVKHKPKATKEQLKAVMDDFAAFVEKCCKADDKETCFAEEGKKLVA
ASQAALGL.

TANZEUM™ is an albiglutide formulation for subcutaneous injection.

v. Dulaglutide

Dulaglutide is GLP-1 receptor agonist that includes a dipeptidyl peptidase-IV-protected GLP-1 analogue covalently linked to a human IgG4-Fc heavy chain by a small peptide linker. The amino acid sequence for dulaglutide is:

(SEQ ID NO: 8)
HGEGTFTSDVSSYLEEQAAKEFIAWLVKGGGGGGGSGGGGSGGGGSAES
KYGPPCPPCPAPEAAGGPSVFLFPPKPKDTLMISRTPEVTCVVVDVSQE
DPEVQFNWYVDGVEVHNAKTKPREEQFNSTYRVVSVLTVLHQDWLNGKE
YKCKVSNKGLPSSIEKTISKAKGQPREPQVYTLPPSQEEMTKNQVSLTC
LVKGFYPSDIAVEWESNGQPENNYKTTPPVLDSDGSFFLYSRLTVDKSR
WQEGNVFSCSVMHEALHNHYTQKSLSLSLG.

TRULICITY is a dulaglutide formulation for subcutaneous injection.

vi. Semaglutide

Semaglutide is GLP-1 analogue that differs to others in the following ways: amino acid substitutions at position 8 (alanine to alpha-aminoisobutyric acid, a synthetic amino acid) and position 34 (lysine to arginine), and acylation of the peptide backbone with a spacer and C-18 fatty di-acid chain to lysine at position 26. These changes permit a high-affinity albumin binding and stabilize semaglutide against dipeptidylpeptidase-4, giving it a long plasma half-life.

The amino acid sequence for semaglutide is:

HXEGTFTSDVSSYLEGQAAKEFIAWLVRGRG (SEQ ID NO:9), where X is alpha-aminoisobutyric acid and Lys20 is acylated with C-18 stearic diacid (AEEAc-AEEAc-y-Glu-17-carboxyheptadecanoyl).

OZEMPIC® is a semaglutide formulation for subcutaneous injection.

vii. Taspoglutide

Taspoglutide is the 8-(2-methylalanine)-35-(2-methylalanine)-36-L-argininamide derivative of the amino acid sequence 7-36 of human GLP-1. Thus, the sequence of taspoglutide is HXEGTFTSDVSSYLEGQAAKEFIAWLVKXX (SEQ ID NO:10), wherein X2 is 2-methylalanine, X29 is 2-methylalanine, and X30 is L-argininamide.

### 2. Polymers

The nanoparticles contain one or more biocompatible polymers. Exemplary suitable polymers include biodegradable polyesters (e.g., polyhydroxyesters), polyanhydrides, or blends or copolymers thereof. Exemplary polymers include poly(lactic acid), poly(glycolic acid), and poly(lactic-co-glycolic acid).

The nanoparticles can contain one or a mixture of two or more polymers. The polymers may be used alone, as physical mixtures (blends), or as co-polymers. The nanoparticles may contain other entities such as stabilizers, surfactants, or lipids.

The nanoparticles can contain one more of the following polyesters: homopolymers including glycolic acid units, referred to herein as “PGA”, and lactic acid units, such as poly-L-lactic acid, poly-D-lactic acid, poly-D,L-lactic acid, poly-L-lactide, poly-D-lactide, and poly-D,L-lactide, collectively referred to herein as “PLA”, and caprolactone units, such as poly(e-caprolactone), collectively referred to herein as “PCL”; and copolymers including lactic acid and glycolic acid units, such as various forms of poly(lactic acid-co-glycolic acid) and poly(lactide-co-glycolide) characterized by the ratio of lactic acid: glycolic acid, collectively referred to herein as “PLGA”; and polyacrylates, and derivatives thereof.

Copolymers including lactic acid and glycolic acid units, such as various forms of poly(lactic acid-co-glycolic acid) and poly(lactide-co-glycolide) can be characterized by the ratio of lactic acid: glycolic acid, collectively referred to herein as “PLGA”; and polyacrylates, and derivatives thereof. In some embodiments, the nanoparticles do not solely contain carboxyl terminated poly(lactide-co-glycolide). However, the nanoparticles may contain a blend of polymers, wherein one of the polymers in the blend is PLGA or carboxyl terminated PLGA.

The polyanhydrides can be formed from the polymerization of dicarboxylic acids. The dicarboxylic acids can be linear saturated dicarboxylic acids or linear unsaturated dicarboxylic acids. Suitable polyanhydrides include polyadipic anhydride, polyfumaric anhydride, polysebacic anhydride, polymaleic anhydride, polymalic anhydride, polyphthalic anhydride, polyisophthalic anhydride, polyaspartic anhydride, polyterephthalic anhydride, polyisophthalic anhydride, poly carboxyphenoxypropane anhydride and copolymers with other polyanhydrides at different mole ratios. For example, a copolymer could contain a first polyanhydride and a second polyanhydride at molar ratios ranging from 5:95 to 95:5, 20:80 to 80:20; or 30:70 to 70:30.

Exemplary polymers also include copolymers of polyethylene glycol (PEG) and the aforementioned polyesters, such as various forms of PLGA-PEG, PGA-PEG, or PLA-PEG copolymers, collectively referred to herein as “PEGylated polymers”. In certain embodiments, the PEG region can be covalently associated with polymer to yield “PEGylated polymers” by a cleavable linker.

Optionally, the polymer does not have PEG conjugated thereto. Optionally, the polymer does not have a terminal carboxylic acid, or corresponding salt thereof.

a. Molecular Weight of Polymer

The polymer forming the nanoparticles disclosed herein can have any suitable molecular weight, such as a molecular weight in a range from about 2 kDa to about 100 kDa, from about 2 kDa to about 50 kDa, from about 2 kDa to about 20 kDa, from about 2 kDa to about 10 kDa, from about 2 kDa to about 5 kDa, from about 4 kDa to about 100 kDa, from about 4 kDa to about 50 kDa, from about 4 kDa to about 20 kDa, from about 4 kDa to about 15 kDa, from about 7 kDa to about 100 kDa, from about 7 kDa to about 50 kDa, from about 7 kDa to about 20 kDa, such as from about 4 kDa to about 15 kDa, from about 7 kDa to about 17 kDa, or about 2 kDa.

When the polymer forming the nanoparticles is a copolymer containing more than one type of monomer, the monomers can have any suitable weight ratios. For example, when the polymer is PLGA, the weight ratio between lactic acid and glycolic acid in the PLGA can be in a range from 1:100 to 100:1, from 10:90 to 90:10, from 20:80 to 80:20, or from 25:75 to 75:25, such as 50:50 or 75:25.

In some embodiments, the polymer forming the nanoparticles containing peptides is PLGA. The PLGA can have a molecular weight in a range from about 1 kDa to about 20 kDa, from about 1 kDa to about 3 kDa, from about 4 kDa to about 15 kDa, or from about 7 kDa to about 17 kDa, such as about 20 kDa, about 50 kDa, or about 100 kDa. The weight ratio of lactic acid to glycolic acid in the PLGA can be in a range from 25:75 to 75:25, such as 50:50 or 75:25.

b. Bioadhesive Polymers

In some embodiments, particularly when the nanoparticles are included in oral formulations, the polymer is optionally a bioadhesive polymer. A bioadhesive polymer is one that binds to mucosal epithelium under normal physiological conditions. Bioadhesion in the gastrointestinal tract proceeds in two stages: (1) viscoelastic deformation at the point of contact of the synthetic material into the mucus substrate, and (2) formation of bonds between the adhesive synthetic material and the mucus or the epithelial cells. In general, adhesion of polymers to tissues may be achieved by (i) physical or mechanical bonds, (ii) primary or covalent chemical bonds, and/or (iii) secondary chemical bonds (i.e., ionic). Physical or mechanical bonds can result from deposition and inclusion of the adhesive material in the crevices of the mucus or the folds of the mucosa. Secondary chemical bonds, contributing to bioadhesive properties, consist of dispersive interactions (i.e., van der Waals interactions) and stronger specific interactions, which include hydrogen bonds. The hydrophilic functional groups primarily responsible for forming hydrogen bonds are the hydroxyl and the carboxylic groups.

Representative bioadhesive polymers include bioerodible hydrogels, such as those described by Sawhney, et al., in Macromolecules, 1993, 26:581-587, the teachings of which are incorporated herein by reference. Other suitable bioadhesive polymers are described in U.S. Pat. No. 6,235,313 to Mathiowitz et al., the teachings of which are incorporated herein by reference, and include polyhydroxy acids, such as poly(lactic acid), polyhyaluronic acids, casein, gelatin, glutin, polyanhydrides, polyacrylic acid, alginate, chitosan; poly(fumaric-co-sebacic) acid, poly(bis carboxy phenoxy propane-co-sebacic anhydride), polyorthoesters, and combinations thereof.

Suitable polyanhydrides include polyadipic anhydride, polyfumaric anhydride, polysebacic anhydride, polymaleic anhydride, polymalic anhydride, polyphthalic anhydride, polyisophthalic anhydride, polyaspartic anhydride, polyterephthalic anhydride, polyisophthalic anhydride, poly carboxyphenoxypropane anhydride and copolymers with other polyanhydrides at different mole ratios.

Optionally, the polymer is a blend of hydrophilic polymers and bioadhesive hydrophobic polymers. Suitable hydrophilic polymers include, but are not limited to, hydroxypropylmethylcellulose, hydroxypropylcellulose, carboxymethylcellulose, polyvinylalcohols, polyvinylpyrollidones, and polyethylene glycols. The hydrophobic polymer may contain gastrosoluble polymers that dissolve in stomach contents, such as Eudragit® E100. The hydrophobic polymer may contain entero-soluble materials that dissolve in the intestine above pH 4.5, such as Eudragit® L-100, Eudragit® S-100, cellulose acetate phthalate, hydroxypropyl methylcellulose phthalate, hydroxypropyl methylcellulose acetate succinate, Eastacryl® 30D dispersion from Eastman Chemicals., Sureteric® (polyvinyl acetate phthalate) and Acryl Eze®.

In some embodiments, the bioadhesive material is a polymer containing a plurality of aromatic groups containing one or more hydroxyl groups. Such polymers are described in detail in U.S. Patent Application Publication No. 2005/0201974 to Schestopol, et al., the disclosure of which is incorporated herein by reference. Suitable aromatic moieties include, but are not limited to, catechol and derivatives thereof, trihydroxy aromatic compounds, or polyhydroxy aromatic moieties. In one embodiment, the aromatic moiety is 3,4-dihydroxyphenylalanine (DOPA), tyrosine, or phenylalanine, all of which contain a primary amine. In a preferred embodiment, the aromatic compound is 3,4-dihydroxyphenylalanine.

The degree of substitution by the aromatic moiety can vary based on the desired adhesive strength; it may be as low as 10%, 20%, 25%, 50%, or up to 100% substitution. On average at least 50% of the monomers in the polymeric backbone are substituted with the at least one aromatic moiety. Preferably, 75-95% of the monomers in the backbone are substituted with at least one of the aromatic groups or a side chain containing one or more aromatic groups. In the preferred embodiment, on average 100% of the monomers in the polymeric backbone are substituted with at least one of the aromatic groups or a side chain containing one or more of the aromatic groups.

The bioadhesive polymer can be formed by first coupling the aromatic compound to a monomer or monomers and polymerizing the monomer or monomers to form the bioadhesive polymer. In this embodiment, the monomers may be polymerized to form any polymer, including biodegradable and non-biodegradable polymers. Alternatively, polymer backbones can be modified by covalently attaching the aromatic moieties to the polymer backbone. In those embodiments where the aromatic moieties are grafted to a polymer chain, the aromatic moieties can be part of a compound, side chain oligomer, and/or polymer.

Regardless of the mechanism, the monomer or polymer generally contains one or more reactive functional groups which can react with the aromatic moiety to form a covalent bond. In one embodiment, the aromatic moiety contains an amino group and the monomer or polymer contains one or more amino reactive groups. Suitable amino reactive groups include, but are not limited to, aldehydes, ketones, carboxylic acid derivatives, cyclic anhydrides, alkyl halides, acyl azides, isocyanates, isothiocyanates, and succinimidyl esters.

The polymer that forms that backbone of the bioadhesive material containing the aromatic groups may be any non-biodegradable or biodegradable polymer.

Suitable polymer backbones include, but are not limited to, polyanhydrides, polyamides, polycarbonates, polyalkylenes, polyalkylene oxides such as polyethylene glycol,, polyvinyl alcohols, polyvinyl ethers, polyvinyl esters, polyethylene, and copolymers thereof, modified celluloses, alkyl cellulose, hydroxyalkyl celluloses, cellulose ethers, cellulose esters, nitro celluloses, polymers of acrylic and methacrylic esters, methyl cellulose, ethyl cellulose, hydroxypropyl cellulose, hydroxy-propyl methyl cellulose, hydroxybutyl methyl cellulose, cellulose acetate, cellulose propionate, cellulose acetate butyrate, cellulose acetate phthalate, carboxylethyl cellulose, cellulose triacetate, and cellulose sulfate sodium salt.

Exemplary biodegradable polymers include synthetic polymers such as poly hydroxy acids, such as polymers of lactic acid and glycolic acid, polyanhydrides, poly(ortho) esters, polyesters, polyurethanes, poly(butic acid), poly(valeric acid), poly(caprolactone), poly(hydroxybutyrate), poly(lactide-co-glycolide) and poly(lactide-co-caprolactone), and natural polymers such as alginate and other polysaccharides, collagen, chemical derivatives thereof (substitutions, additions of chemical groups, for example, alkyl, alkylene, hydroxylations, oxidations, and other modifications routinely made by those skilled in the art), albumin and other hydrophilic proteins, zein and other prolamines and hydrophobic proteins, copolymers and mixtures thereof. In general, these materials degrade either by enzymatic hydrolysis or exposure to water in vivo, by surface or bulk erosion. The foregoing materials may be used alone, as physical mixtures (blends), or as co-polymers.

c. Bioadhesive Oligomers

Polymers with enhanced bioadhesive properties can be provided wherein bioadhesive monomers or oligomers, such as anhydride monomers or oligomers, are incorporated into the polymer. The oligomer excipients can be blended or incorporated into a wide range of hydrophilic and hydrophobic polymers including proteins, polysaccharides and synthetic biocompatible polymers. Anhydride oligomers may be combined with metal oxide particles to improve bioadhesion even more than with the organic additives alone. The incorporation of oligomer compounds into a wide range of different polymers which are not normally bioadhesive can increase their adherence to tissue surfaces, such as mucosal membranes.

As used herein, the term “anhydride oligomer” refers to a diacid or polydiacids linked by anhydride bonds, and having carboxy end groups linked to a monoacid such as acetic acid by anhydride bonds. The anhydride oligomers have a molecular weight less than about 5000, typically between about 100 and 5000 Da, or are defined as including between one to about 20 diacid units linked by anhydride bonds. In one embodiment, the diacids are those normally found in the Krebs glycolysis cycle. The anhydride oligomer compounds have high chemical reactivity.

The oligomers can be formed in a reflux reaction of the diacid with excess acetic anhydride. The excess acetic anhydride is evaporated under vacuum, and the resulting oligomer, which is a mixture of species which include between about one to twenty diacid units linked by anhydride bonds, is purified by recrystallizing, for example from toluene or other organic solvents. The oligomer is collected by filtration, and washed, for example, in ethers. The reaction produces anhydride oligomers of mono and poly acids with terminal carboxylic acid groups linked to each other by anhydride linkages.

The anhydride oligomer may be hydrolytically labile. As analyzed by gel permeation chromatography, the molecular weight may be, for example, on the order of 200 to 400 for fumaric acid oligomer (FAPP) and 2000 to 4000 for sebacic acid oligomer (SAPP). The anhydride bonds can be detected by Fourier transform infrared spectroscopy by the characteristic double peak at 1750 cm 1 and 1820 cm 1, with a corresponding disappearance of the carboxylic acid peak normally at 1700 cm 1.

In one embodiments, the oligomers may be made from diacids described for example in U.S. Pat. No. 4,757,128 to Domb et al., U.S. Pat. No. 4,997,904 to Domb, and U.S. Pat. No. 5,175,235 to Domb et al., the disclosures of which are incorporated herein by reference. For example, monomers such as sebacic acid, bis(p carboxy phenoxy) propane, isophathalic acid, fumaric acid, maleic acid, adipic acid or dodecanedioic acid may be used.

d. Bioadhesive Additives

Additives can be added to the polymer to alter the properties of the polymer provided the additives do not adversely affect the formation of the nanoparticles. Suitable additives include, but are not limited to, dyes and excipients which alter porosity, permeability, hydration, and/or disintegration properties.

Organic dyes because of their electronic charge and hydrophobicity/hydrophilicity can be used to either increase or decrease the bioadhesive properties of polymers when incorporated into the polymer. Suitable dyes include, but are not limited to, acid fuchsin, alcian blue, alizarin red s, auramine o, azure a and b, Bismarck brown y, brilliant cresyl blue ald, brilliant green, carmine, cibacron blue 3GA, congo red, cresyl violet acetate, crystal violet, eosin b, eosin y, erythrosin b, fast green fcf, giemsa, hematoylin, indigo carmine, Janus green b, Jenner's stain, malachite green oxalate, methyl blue, methylene blue, methyl green, methyl violet 2b, neutral red, Nile blue a, orange II, orange G, orcein, paraosaniline chloride, phloxine b, pyronin b and y, reactive blue 4 and 72, reactive brown 10, reactive green 5 and 19, reactive red 120, reactive yellow 2,3, 13 and 86, rose bengal, safranin o, Sudan III and IV, Sudan black B and toluidine blue.

The bioadhesives properties can also be improved by adding metal compounds, such as water-insoluble metal oxides and metal hydroxides, which are capable of becoming incorporated into and associated with a polymer to thereby improve the bioadhesiveness of the polymer as described in U.S. Pat. No. 5,985,312, which is incorporated herein by reference in its entirety. As defined herein, a water-insoluble metal compound is defined as a metal compound with little or no solubility in water, for example, less than about 0.0-0.9 mg/ml.

The water-insoluble metal compounds, such as metal oxides, can be incorporated by one of the following mechanisms: (a) physical mixtures which result in entrapment of the metal compound; (b) ionic interaction between metal compound and polymer; (c) surface modification of the polymers which would result in exposed metal compound on the surface; and (d) coating techniques such as fluidized bead, pan coating or any similar methods known to those skilled in the art, which produce a metal compound enriched layer on the surface of the device.

The water-insoluble metal compounds can be derived from metals including calcium, iron, copper, zinc, cadmium, zirconium and titanium. For example, a variety of water-insoluble metal oxide powders may be used to improve the bioadhesive properties of polymers such as ferric oxide, zinc oxide, titanium oxide, copper oxide, barium hydroxide, stannic oxide, aluminum oxide, nickel oxide, zirconium oxide and cadmium oxide. The incorporation of water-insoluble metal compounds such as ferric oxide, copper oxide and zinc oxide can tremendously improve adhesion of the polymer to tissue surfaces such as mucosal membranes, for example in the gastrointestinal system. The polymers incorporating a metal compound thus can be used to form or coat the nanoparticles to improve their bioadhesive properties.

### 3. Peptide Release Profiles

The nanoparticles disclosed herein can provide sustained release of the peptide with less than 20% of the peptide released initially (at time 0 hour) following placement into a phosphate buffered saline at pH 7.4 at 37° C. and room pressure. In some embodiments, the nanoparticles can release less than 50% of the peptide at 24 hours following placement into the phosphate buffered saline. In some embodiments, the nanoparticles can release less than 50% of the peptide at 200 hours following placement into the phosphate buffered saline. In some embodiments, the nanoparticles can release ≥50% of the peptide at or after 400 hours following placement into the phosphate buffered saline. As discussed below, the release of peptides at 24 hours following placement into the phosphate buffered saline is primarily by diffusion through pores on and/or in the nanoparticles, while release of peptides at 200 hours and 400 hours is generally due to polymer degradation that exposes the encapsulated peptides.

Peptide release behavior from the polymeric nanoparticles can be divided into four categories: burst release, diffusion release, lag time, and degradation release. The initial burst release, such as the amount of peptide released at time 0 hr, generally relates to the release of poorly encapsulated peptide located on the surface of the nanoparticles, which is immediately released upon suspension of the particles. In cases where sustained release is desired, burst is undesirable as it can result in a loss of peptide content or toxic high dosages. Following the initial burst, peptides may diffuse from the polymeric nanoparticles through a network of pores. While peptides may not directly diffuse through the polymer matrix, they may release through pores left behind by the surface encapsulated peptides from the initial burst. Deeper encapsulated peptides are thought to diffuse through the free volume of empty pores left behind by peptide released closer to the surface, diffusing though a network of pores. In the absence of this network of pores, the peptides may remain encapsulated by the polymers and result in a lag time. Lag time refers to a period in which no peptide release occurs, between the initial burst/diffusion and the onset of polymer degradation. For example, in the case of PLGA nanoparticles encapsulating GLP-1 RA, PLGA eventually begins to degrade by hydrolysis. Upon degradation, a second phase of GLP-1 RA release occurs, in which the encapsulated peptide is exposed as the polymer chains break down.

As described below, a desired release profile of peptides from the nanoparticles can be achieved by selecting the appropriate ΔG_{Mix} of the solvent and non-solvent pairs used to form the nanoparticles encapsulating peptides. For example, when the desired release profile of the formed nanoparticles is sustained release over a long period of time, such as a lag time period ≥200 hours, without an initial burst release (i.e. ≤20% release of theoretical loading at time 0 hr), then the solvent and non-solvent pairs for forming the nanoparticles are selected to have a more negative ΔG_{Mix}, such as equalto or more negative than −0.6 (i.e. less than or equal to about −0.6, such as less than −0.8, less than −1.0, such as −1.2). When the desired release profile of the formed nanoparticles is rapid release over a short period of time, such as a lag time period ≤24 hours, with burst release (i.e. >20% of theoretical loading at time 0 hr), then the ΔG_{Mix} of the solvent and non-solvent pairs for forming the nanoparticles should be less negative, such as less negative than −0.6 (i.e. greater than −0.6, such as −0.2).

### B. Pharmaceutical Compositions or Formulations

Pharmaceutical compositions or formulations that contain the polymeric nanoparticles described herein in a form suitable for administration to a mammal are disclosed. For example, the pharmaceutical composition or formulation containing the disclosed nanoparticles can be in a liquid form or a solid form. The pharmaceutical composition or formulation containing the disclosed nanoparticles can be in any suitable form for delivery by the desired method, such as oral, intraperitoneal administration, nasal administration, injection (intraperitoneal, subcutaneous, intramuscular, intravenous), sublingual, inhalation, and transdermal delivery.

The pharmaceutical composition or formulation may include one or more pharmaceutically acceptable carriers and/or one or more pharmaceutically acceptable excipients. For example, the pharmaceutical formulation may be in the form of a liquid, such as a solution or a suspension, and contain the disclosed nanoparticles in an aqueous medium and, optionally, one or more suitable excipients for the liquid formulation. For example, if the liquid formulation is in an aqueous medium as an aqueous solution for administration, the nanoparticles are dissolved or suspended in the aqueous medium just before use to prevent degradation of the polymer. When the liquid formulation is in an oil medium, such oil formulation of nanoparticles can be stored and stable for a longer period of time, such as one day, three days, one week, 2 weeks, 1 month, 3 months, 6 months, etc. at room temperature or in a refrigerator. Optionally, the pharmaceutical composition or formulation is in a solid form, and contains the nanoparticles and one or more suitable excipients for a solid formulation.

The pharmaceutical composition or formulation can contain one or more pharmaceutically acceptable carriers and/or excipients. Suitable pharmaceutically acceptable carriers and excipients are generally recognized as safe (GRAS), and may be administered to an individual without causing undesirable biological side effects or unwanted interactions.

Representative carriers and excipients include solvents (including buffers), diluents, pH modifying agents, preservatives, antioxidants, suspending agents, wetting agents, viscosity modifiers, tonicity agents, and stabilizing agents, and a combination thereof.

Nanoparticles for delivering peptides to the blood circulation or a site of the mammal can be dissolved or suspended in a suitable carrier to form a liquid pharmaceutical formulation, such as sterile saline, phosphate buffered saline (PBS), balanced salt solution (BSS), viscous gel, or other pharmaceutically acceptable carriers for administration. The pharmaceutical composition or formulation may also be a sterile solution, suspension, or emulsion in a nontoxic, parenterally acceptable diluent or solvent. For example, formulations containing the polymeric nanoparticles disclosed herein are in a solid form and stored dry to avoid degradation of the polymer. At the time of use or shortly before us, the dry nanoparticles are dissolved or suspended in a suitable aqueous medium.

Excipients can be added to a liquid or solid pharmaceutical formulation to assist in sterility, stability (e.g. shelf-life), integration, and to adjust and/or maintain pH or isotonicity of the nanoparticles in the pharmaceutical composition or formulation, such as diluents, pH modifying agents, preservatives, antioxidants, suspending agents, wetting agents, viscosity modifiers, tonicity agents, and stabilizing agents, and a combination thereof.

### II. Methods of Micronizing Peptides

Methods for micronizing a peptide, such as insulin or a GLP-1 RA, are also disclosed. Examples of additional peptides that can be micronized using the methods described herein include, but are not limited to, glucagon, pramlintide, insulin, leuprolide, an luteinizing-hormone-releasing hormone (LHRH) agonist, parathyroid hormone (PTH) or its pharmaceutically active sub-units, amylin, botulinum toxin, hematide, an amyloid peptide, cholecystikinin, gastric inhibitory peptide, an insulin-like growth factor, growth hormone releasing factor, anti-microbial factor, glatiramer, glucagon-like peptide-1 (GLP-1), a GLP-1 agonist, e.g., exenatide, interferons, insulin, insulin analogs, c-peptide, amylin, analogues thereof, and mixtures thereof. The micronization methods disclosed herein can retain the bioactivity of the peptides being micronized, even with the exposure to organic solvents.

Generally, the method includes (i) dissolving the peptide in an effective amount of a peptide solvent, wherein the peptide and the peptide solvent to form a peptide solution, and (ii) introducing the peptide solution into a peptide non-solvent. Typically, the peptide solvent and the peptide non-solvent are miscible, i.e., the Gibbs energy of mixing (ΔGₘᵢₓ/RT) for the peptide solvent and the peptide non-solvent is negative. For example, the Gibbs energy of mixing (ΔGₘᵢₓ/RT) for the peptide solvent and the peptide non-solvent is less than or equal to about −0.6.

The peptide solvent and the peptide non-solvent can have any suitable volume ratio, as long as the (ΔGₘᵢₓ/RT) for the pair has a desired value. For example, the volume ratio of peptide solvent: peptide non-solvent can be in a range from 1:1000 to 1000 to 1, from 1:500 to 500 to 1, from 1:200 to 200 to 1, from 1:100 to 100 to 1, from 1:50 to 50 to 1, from 1:20 to 20 to 1, from 1:10 to 10 to 1, or from 1:5 to 5 to 1, such as about 1:60. The peptide in the peptide solution during step (i) can have any suitable concentration. For example, the concentration of the peptide is in a range from about 0.1 mg/mL to about 200 mg/mL, from about 1 mg/mL to about 100 mg/mL, from about 5 mg/mL to about 50 mg/mL, or from about 10 mg/mL to about 30 mg/mL, such as about 20 mg/mL, in the peptide solution.

In some embodiments, during step (ii), the peptide is precipitated to produce a composition of micronized nanoparticles of peptide. Generally, the micronized peptide nanoparticles have a number average size of less than 5 microns, less than 4 microns, less than 3 microns, less than 2 microns, less than 1 micron, such as in a range from about 10 nm to about 1 micron, from about 10 nm to about 500 nm, from about 10 nm to about 400 nm, from about 10 nm to about 300 nm, from about 50 nm to about 500 nm, from about 50 nm to about 400 nm, from about 50 nm to about 300 nm, from about 50 nm to about 250 nm, from about 100 nm to about 500 nm, from about 100 nm to about 400 nm, or from about 100 nm to about 300 nm. For example, the micronized peptide nanoparticles have a number average size of about 300 nm or less, such as in a range from about 10 nm to about 300 nm, from about 50 nm to about 300 nm, or from about 100 nm to about 300 nm.

The peptide solvent and peptide non-solvent for micronizing the peptide can be any suitable solvents, such as water and organic solvents. The organic solvent, when used as the peptide non-solvent, can be polar or non-polar, and protic or aprotic. In some embodiments, the organic solvent, when used as the peptide non-solvent, is protic. For example, peptide solvents suitable for micronizing the peptide can be methanol or water, or a combination thereof. Examples of peptide non-solvent include, but are not limited to, tert-butanol, 2-propanol, acetonitrile, acetone, ethyl acetate, tetrahydrofuran, dichloromethane, and chloroform, and a combination thereof. In some forms, the peptide solvent and peptide non-solvent pair for micronizing the peptide is tert-butanol-methanol, 2-propanol-methanol, acetonitrile-methanol, acetone-methanol, dicholormethane-methanol, chloroform-methanol, ethyl acetate-methanol, or tetrahydrofuran-methanol.

The micronization and characterization of GLP-1 RA peptide nanoparticles are exemplified below. Specifically, a phase inversion process was used to micronize peptides. While the Examples refer to micronized GLP-1 RA nanoparticles, similar results are expected with other peptides, particularly peptides having a molecular weight of less than about 6000 Da. Data is provided to show the mechanism of micronized peptide nanoparticle formation by supersaturation, nucleation, and growth upon adding a peptide solution to a miscible peptide non-solvent. This mechanism is correlated to the Gibbs energy of mixing (ΔG_{Mix}) between the solvent and non-solvent used in the phase inversion process, calculated using the activity coefficients obtained from the UNIFAC method.

By choosing the peptide solvent and peptide non-solvent pairs having appropriate Gibbs energy of mixing (ΔGₘᵢₓ/RT) to micronize the peptide, properties of the formed micronized peptide nanoparticles, such as size of the peptide nanoparticles, can be tuned. For example, the size of micronized GLP-1 RA nanoparticles decreases with increasingly negative ΔG_{Mix} of the peptide solvent and peptide non-solvent pairs used to produce them. It is noted that when ΔG_{Mix} is discussed (unless otherwise indicated, such as in the equation), it is referred to the value of ΔG_{Mix}/RT, where R is the universal gas constant and Tis the temperature in Kelvin. For example, for producing micronized a micronized peptide, such as GLP-1RA peptide, of <550 nm under the micronization conditions describe in the Examples below, the solvent non-solvent pair is selected to have a ΔGₘᵢₓ/RT that is more negative than −0.333. In these forms, any suitable solvents and non-solvents for micronizing peptides can be used that achieve a ΔGₘᵢₓ/RT that is more negative than −0.333 (i.e. less than-0.333). Exemplary peptide solvent-peptide non-solvent pairs suitable for achieving such a ΔGₘᵢₓ/RT include, but are not limited to, tert-butanol-methanol, 2-propanol-methanol, acetonitrile-methanol, acetone-methanol, dicholormethane-methanol, chloroform-methanol, ethyl acetate-methanol, and tetrahydrofuran-methanol.

Thus, the micronization method described herein can be used for micronizing any suitable peptides to achieve a desired nanoparticle size while retaining their bioactivities.

### A. UNIFAC Gibbs Energy Calculations

The Gibbs energy of mixing between solvents and non-solvents were calculated using the UNIFAC method.

Briefly, the Gibbs energy of mixing for an ideal mixture is defined as:

$$Δ {G}^{Ideal} = - RT \sum {x}_{i} Ln ( {x}_{i} )$$

Where, R is the gas constant, T is temperature (Kelvin), and xi is the mole fraction of each component (Prausnitz, J., Lichtenthaler, R. & Gomes de Azevedo, E. Molecular Thermodynamics of Fluid-Phase Equilibria. (Prentice Hall PTR, 1999)). However, mixtures of real fluids typically only behave close to ideal when each component has similar properties (as ideal behavior assumes the interactions between all components are the same). For example, in an ideal binary mixture of component A and component B, there is no distinguishing between A-A, B-B, and A-B interactions. For this reason, the UNIFAC method was adopted for estimating the non-ideal Gibbs energy of mixing between solvents used in the PIN process.

The UNIFAC method aids in describing deviations from ideal solution behavior through the calculation of excess functions. Excess functions contain activity coefficients that give a quantitative measure of interactions between individual components within a mixture (i.e. A-A, B-B, and A-B interactions). The non-ideal behavior of the Gibbs energy of mixing between real fluids then is accounted for by an excess Gibbs energy term:

$${G}_{E} = RT \sum {x}_{i} Ln ( {γ}_{i} )$$

Where γᵢ is the activity coefficient determined by the UNIFAC method. Thus, the non-ideal Gibbs energy of mixing is calculated by:

$$Δ {G}_{Mix} = Δ {G}_{Ideal} + {G}_{E} = RT \sum {x}_{i} Ln ( {x}_{i} * {γ}_{i} )$$

The UNIFAC method was first put forth in 1977 in a publication by Aage Fredenslund, Jürgen Gmehling, and Peter Rasmussen (Fredenslund, A., Gmehling, J. & Rasmussen, P. Vapor-liquid Equilibria Using Unifac. (Elsevier, 1977). doi: 10.1016/B978-O-444-41621-6.X5001-7). The UNIFAC method is a semi-empirical method based on the universal quasichemical (UNIQUAC) method for calculating activity coefficients. Accordingly, UNIFAC is an abbreviation for the UNIQUAC Function Group Activity Coefficents (UNIFAC) method. Through the analysis of each component's functional groups, the semi-empirical nature of the UNIFAC method allows for estimations of activity coefficients without any experimental data needed.

TABLE 1
List of functional groups used to describe each solvent
in the UNIFAC calculations of Gibbs energy

| Solvent | Functional Groups |
|---|---|
| Dichloromethane | 1-CH₂Cl₂ |
| Chloroform | 1-CCl₃ |
| Acetonitrile | 1-CH₃CN |
| Acetone | 1-CH₃, 1-CH₃CO |
| Ethyl Acetate | 1-CH₃COO, 1-CH₂, 1-CH₃ |
| Tert-Butanol | 3-CH₃, 1-OH |
| 2-Propanol | 1-(CH₃)₂CHOH |
| Ethanol | 1-CH₃, 1-CH₂, 1-OH |
| Heptane | 2-CH₃, 5-CH₂ |
| Water | 1-H₂O |

Table 1 lists the functional groups used to describe each solvent in calculations. All Gibbs energy calculations were done using an Excel sheet provided by Lira and Elliott as a digital supplement to the Introductory Chemical Engineering Thermodynamics textbook (Lira, C. & Elliott, R. Introductory Chemical Engineering Thermodynamics. (Pearson, 2012)).

### III. Methods for Forming Nanoparticles Containing Micronized Peptides

Methods for forming nanoparticles containing solid, micronized peptides are disclosed. Generally, the method includes: (a) dissolving a polymer in a first suspension containing the solid micronized peptide and a polymer solvent to form a second suspension, and (b) introducing the second suspension into a polymer non-solvent to spontaneously form the nanoparticle. The polymer non-solvent is also a non-solvent for the solid, micronized peptide. In some embodiments, step (b) does not include emulsification, agitation, and/or stirring.

Typically, the polymer solvent and the polymer non-solvent are miscible, i.e., the Gibbs energy of mixing (ΔGₘᵢₓ/RT) for the polymer solvent and the polymer non-solvent is negative. For example, the Gibbs energy of mixing (ΔGₘᵢₓ/RT) for the polymer solvent and the polymer non-solvent is less than or equal to about −0.6.

The polymer solvent and the polymer non-solvent can have any suitable volume ratio, as long as the (ΔGₘᵢₓ/RT) for the pair has a desired value. For example, the volume ratio of polymer solvent: polymer non-solvent can be in a range from 1:1000 to 1000 to 1, from 1:500 to 500 to 1, from 1:200 to 200 to 1, from 1:100 to 100 to 1, from 1:50 to 50 to 1, from 1:20 to 20 to 1, from 1:10 to 10 to 1, or from 1:5 to 5 to 1, such as about 1:60.

The polymer is soluble in the polymer solvent and thus after dissolving in the polymer solvent, the polymer can have a suitable concentration in the second suspension for forming the nanoparticles. For example, the concentration of the polymer is in a range from about 1 mg/mL to about 1000 mg/mL, from about 10 mg/mL to about 500 mg/mL, or from about 50 mg/mL to about 200 mg/mL, such as about 100 mg/mL, in the second suspension.

The preparation and characterization of PLGA nanoparticles loaded with a GLP-1 RA peptide is exemplified below. Here, a phase inversion process was used to micronize peptides and a phase inversion nanoencapsulation process was used to prepare polymeric nanoparticles encapsulating the micronized peptides. While the Examples refer to GLP-1 RA and PLGA nanoparticles, similar results are expected with other peptides and/or other polymers, particularly biocompatible, biodegradable polymers, such as any one of those described above. Data is provided to show the mechanism of particle formation by supersaturation, nucleation, and growth upon adding a solubilized polymer or drug solution to a miscible non-solvent. This mechanism is correlated to the Gibbs energy of mixing (ΔG_{Mix}) between the solvent and non-solvent used in the phase inversion process, calculated using the activity coefficients obtained from the UNIFAC method.

By choosing the polymer solvent and polymer non-solvent pairs having appropriate ΔG_{Mix} for the phase inversion process, it is demonstrated that particle size, encapsulation efficiency, and release profile can be modified and tailored to prepare polymeric nanoparticles having desired release properties, such as no or low burst release and sustained release of the encapsulated or dispersed agent (e.g., peptide). For example, the size of polymeric nanoparticles decreases with increasingly negative ΔG_{Mix} of the polymer solvent and polymer non-solvent pairs used to produce them. For example, polymeric nanoparticles formed using ΔG_{Mix} of the polymer solvent and polymer non-solvent pairs that is more negative, such as equals to or more negative than −0.6, the nanoparticles can release the peptide encapsulated or dispersed therein in a sustained manner, without burst release (such as 0-20% of the theoretical loading at time 0 hr and majority of the peptide released after an approximate 200 hour lag period). For example, polymeric nanoparticles formed using ΔG_{Mix} of the polymer solvent and polymer non-solvent pairs that is less negative, such as less negative than-0.6, the nanoparticles can release the peptide encapsulated or dispersed therein with a large burst release (such as approximately 20-70% of the theoretical loading at time 0 hr and approximately 60-100% of the theoretical loading released by 24 hour time point).

In some forms, by choosing the polymer solvent and polymer non-solvent pairs having appropriate Gibbs energy of mixing (ΔGₘᵢₓ/RT) to produce the polymeric nanoparticles, size of the polymeric nanoparticles, can be tuned. For example, for producing Resomer® RG 752 H, PLGA (75:25) 4-15 kDa nanoparticles of <500 nm under the PIN conditions described in the Examples below, ΔGₘᵢₓ/RT is selected to be more negative than-0.297 (i.e. less than −0.297). In these forms, any suitable solvents and non-solvents for producing polymeric particles can be that achieve a ΔGₘᵢₓ/RT that is more negative than-0.297. Exemplary polymer solvent-polymer non-solvent pairs suitable for achieving such a ΔGₘᵢₓ/RT for polymers such as PLGA (75:25) 4-15 kDa, include, but are not limited to, dicholormethane-tertbutanol, dichloromethane-2-propanol, dichloromethane-ethanol, dichloromethane-ethanol, dichloromethane-heptane, chloroform-tertbutanol, chloroform-2-propanol, chloroform-ethanol, chloroform-heptane, ethyl acetate-tertbutanol, ethyl acetate-ethanol, ethyl acetate-heptane, acetone-tertbutanol, acetone-2-propanol, acetone-ethanol, acetonitrile-tertbutanol, acetonitrile-2-propanol, acetonitrile-ethanol, and acetonitrile-water.

The same parameters used for the selection of solvent and non-solvent, described in Section II above with respect to forming micronized peptide nanoparticles, apply to the selection of a polymer solvent and polymer non-solvent when this process is used to form polymeric nanoparticles encapsulating peptides. For example, the polymer solvent and polymer non-solvent each can be polar or non-polar, and protic or aprotic. In some embodiments, the polymer solvent is non-polar. In some forms, the polymer solvent is dichloromethane, chloroform, ethyl acetate, acetone, acetonitrile, or tetrahydrofuran. For example, the polymer solvent is dichloromethane, chloroform, ethyl acetate, or acetonitrile. In some forms, the polymer non-solvent is tert-butanol, 2-propanol, ethanol, heptane, water. In some forms, the polymer solvent and polymer non-solvent pair for producing polymeric nanoparticles encapsulating micronized peptides is dicholormethane-tertbutanol, dichloromethane-2-propanol, dichloromethane-ethanol, dichloromethane-ethanol, dichloromethane-heptane, chloroform-tertbutanol, chloroform-2-propanol, chloroform-ethanol, chloroform-heptane, ethyl acetate-tertbutanol, ethyl acetate-ethanol, ethyl acetate-heptane, acetone-tertbutanol, acetone-2-propanol, acetone-ethanol, acetonitrile-tertbutanol, acetonitrile-2-propanol, acetonitrile-ethanol, or acetonitrile-water.

Generally, the polymeric nanoparticles encapsulating peptides have a number average size of less than 10 microns, less than 8 microns, less than 6 microns, less than 4 microns, less than 2 micron, such as in a range from about 10 nm to about 10 microns, from about 10 nm to about 5 microns, from about 10 nm to about 2 microns, from about 50 nm to about 10 microns, from about 50 nm to about 5 microns, from about 50 nm to about 2 microns, from about 100 nm to about 10 microns, from about 100 nm to about 5 microns, or from about 100 nm to about 2 microns. For example, the polymeric nanoparticles encapsulating peptides have a number average size of less than 10 microns, less than 8 microns, less than 6 microns, less than 4 microns, less than 2 micron, such as in a range from about 10 nm to about 2 microns, from about 50 nm to about 2 microns, or from about 100 nm to about 2 microns.

In some embodiments, the method for forming the polymeric nanoparticles described herein can include (i) micronizing a peptide to form the first suspension comprising the micronized peptide, prior to step (a) dissolving a polymer in a first suspension containing the solid micronized peptide and a polymer solvent to form the second suspension.

In some embodiments, the peptide is micronized using the method described above for micronizing peptides and then encapsulated in polymeric nanoparticles using the method described herein. In these embodiments, the non-solvent for the peptide (i.e. peptide non-solvent) is optionally selected such that it is also the solvent for the polymer (i.e. polymer solvent). Thus, in some embodiments, the peptide non-solvent is the same as the polymer solvent. An advantage of this process is that separation of the micronized peptide after the first micritization process is avoided, thus making the process easier to scale up compared to previous PIN processes, such as that described in U.S. Publication No. 2010/0172998.

In some other embodiments, the peptide is micronized using any suitable method to provide nanoparticles of the peptide, and the nanoparticles of the peptide are dried to produce a powder by filtration and/or lyophilization. A polymer solution is prepared separately and the powder of micronized peptide nanoparticles is added to the polymer solution to form a dispersion that is then added to a polymer non-solvent, see, for example, the method described in U.S. Publication No. 2010/0172998.

The polymer solvent and polymer non-solvent for forming the polymeric nanoparticles encapsulating the peptides can be any suitable solvents, as long as they have an appropriate ΔG_{Mix} for forming the polymeric nanoparticles. For example, polymer solvents can be dichloromethane or chloroform, or a combination thereof. Polymer non-solvents can be 2-propanol or heptane, or a combination thereof.

### IV. Methods of Use

Nanoparticles formed by the methods described herein that encapsulate or have dispersed therein one or more peptides can be formulated into a pharmaceutical formulation or composition. The nanoparticles can be formulated into a variety of different drug delivery dosage forms and administered to a patient by any suitable method, including oral, intraperitoneal administration, nasal administration, injection (intraperitoneal, subcutaneous, intramuscular, intravenous), sublingual, inhalation, and transdermal delivery. Most typically, the compositions are formulated for and/or delivered by oral administration.

The pharmaceutical formulations can be administered to a subject in need of treatment and deliver an effective amount of the peptide encapsulated or dispersed in the nanoparticles for a sustained period of time, such as for at least 1 week, for at least 2 weeks, or for longer than 2 weeks, such as for up to 1 month following administration.

The compositions can be administered in an effective amount to a subject in need thereof. The terms “effective amount” and “therapeutically effective amount” typically means a dosage sufficient to alleviate one or more symptoms of a disorder, disease, or condition being treated, or to otherwise provide a desired pharmacologic and/or physiologic effect. In some embodiments, the composition leads to increased bioavailability, bioactivity, or a combination thereof an active agent relative to a control. In some embodiments, the composition allows the active agent to be used at a lower dosage or less frequent administrations relative to a control.

By way of example, the peptide in the nanoparticles can be GLP-1, a truncated biologically active portion thereof, or an analogue thereof. An effective amount of the pharmaceutical composition can be administered to significantly reduce fasting blood glucose, post-prandial blood glucose, HbA1c, weight, and/or daily insulin requirements (see Gupta, Indian J Endocrinol Metab. 2013 May-Jun.; 17 (3): 413-421.). In some embodiments, a pharmaceutical composition containing polymeric nanoparticles containing GLP-1, or a biologically active portion thereof or an analogue thereof is administered in an effective amount to reduce fasting blood glucose, post-prandial blood glucose, glycated haemoglobin (HbA1c), weight, or daily insulin requirements, or a combination thereof.

GLP-1, a truncated biologically active portion thereof, or an analogue thereof can be administered to treat Type I and Type II diabetes, and have shown a substantial beneficial pleiotropic effect, extending to virtually every organ system. For example, GLP-1 analogues have been shown improve cardiovascular parameters, having a positive effect on myocardial contractility, hypertension (natriuretic/diuretic effect), endothelium (anti-atherosclerotic), and lipid profile (improvement in HDL cholesterol, fasting triglycerides).

GLP-1, a truncated biologically active portion thereof, or an analogue thereof can be administered to facilitate neuronal protection, resulting in an improvement in cognition, memory, and spatial learning. It modifies eating behavior by inducing satiety, thereby reducing energy intake by approximately 12%. Via interaction with the peripheral nervous system (vagus) central, GLP-1 augmentation causes gastric slowing, inducing a post-prandial satiety. Weight loss, which can also be induced by GLP-1 analogues, is dose dependent and progressive.

GLP-1, a truncated biologically active portion thereof, or an analogue thereof can be administered to reduce insulin sensitivity through restoration of insulin signaling and reduction of hepatic gluconeogenesis. Enhanced insulin secretion causes increased uptake of glucose in the muscle and adipocytes, and reduced expression of glucose from the liver. By promoting weight loss, GLP-1 analogues can improve peripheral insulin-mediated glucose uptake. Reduced insulin resistance is evident locally, at the level of beta-cell and fat cell (reduced release of free fatty acids) and systemically (down-gradation of markers of inflammation).

Optionally, a pharmaceutical composition containing polymeric nanoparticles containing GLP-1, a truncated biologically active portion thereof, or an analogue thereof is administered in an effective amount to improve cardiovascular heath, enhance neuroprotection, induce weight loss, reduce insulin sensitivity, or a combination thereof. In some embodiments, the compositions are administered in an effect amount to alter one or more physiological or biochemical parameters or symptoms discussed herein.

The precise dosage of compositions will vary according to a variety of factors such as subject-dependent variables (e.g., age, immune system health, etc.), the disease or disorder being treated, as well as the route of administration and the pharmacokinetics of the agent being administered. Exemplary dosages for subcutaneous administration of common GLP-1 analogues are introduced above and otherwise known in the art. In some embodiments, the dosage of GLP-1, a truncated biologically active portion thereof, or an analogue thereof administered in the disclosed nanoparticle formulations is the same or similar to those mentioned above or known in the art. In some embodiments, the dosage is higher or lower that the art recognized dosage. For example, in some embodiments, the dosage GLP-1, a truncated biologically active portion thereof, or an analogue thereof in a nanoparticle formulation administered orally is the same or higher than a traditional subcutaneous administration without nanoparticles. In some embodiments, the dosage GLP-1, a truncated biologically active portion thereof, or an analogue thereof in a particle formulation administered subcutaneously is the same or lower than a traditional subcutaneous administration without nanoparticles.

The disclosed particles, pharmaceutical formulations, and methods can be further understood through the following numbered paragraphs.
1. A pharmaceutical composition for delivering a peptide comprising a micronized peptide encapsulated or dispersed in a nanoparticle,
wherein the nanoparticle comprises one or more polymers, and
wherein the composition provides sustained release of the peptide with less than 20% of the peptide released initially (0 hour) following placement into a phosphate buffered saline at 37° C. and room pressure (i.e., 1 atm).
2. The pharmaceutical composition of paragraph 1, wherein the peptide has a molecular weight of up to 6,000 Da.
3. The pharmaceutical composition of paragraph 1 or 2, wherein less than 50% of the peptide is released at 24 hours
4. The pharmaceutical composition of paragraph 1 or 2, wherein less than 50% of the peptide is released at 200 hours.
5. The pharmaceutical composition of paragraph 1 or 2, wherein ≥50% of the peptide is released at or after 400 hours.
6. The pharmaceutical composition of any one of paragraphs 1-5, wherein the nanoparticle has a number average size of 10 microns or less, 5 microns or less, 2 microns or less, 750 nm or less, 500 nm or less, or 300 nm or less; and/or wherein the micronized nanoparticles have a number average size of 5 microns or less, 1 micron or less, 300 nm or less.
7. The pharmaceutical composition of any one of paragraphs 1-6, wherein the polymer is a biodegradable polymer, such as a polymer selected from the group consisting of biodegradable polyesters (e.g., polyhydroxyesters), polyanhydrides, poly(lactic acid), poly(glycolic acid), and poly(lactic-co-glycolic acid), or a copolymer thereof, or a mixture thereof.
8. The pharmaceutical composition of any one of paragraphs 1-7, wherein the polymer is PLGA.
9. The pharmaceutical composition of paragraph 8, wherein the PLGA has a molecular weight in a range from about 2 kDa to about 20 kDa, from about 2 kDa to about 3 kDa, from about 4 kDa to about 15 kDa, or from about 7 kDa to about 17 kDa.
10. The pharmaceutical composition of paragraph 8 or 9, wherein the weight ratio of lactic acid to glycolic acid in the PLGA is in a range from 25:75 to 75:25, such as 50:50 or 75:25.
11. The pharmaceutical composition of any one of paragraphs 1-10, wherein the peptide is a glucagon-like peptide-1 receptor agonist (“GLP-1 RA”).
12. A method for micronizing a peptide, comprising:
(i) dissolving the peptide in an effective amount of a peptide solvent to form a peptide solution,
(ii) introducing the peptide solution into a peptide non-solvent,
wherein the peptide solvent and the peptide non-solvent are miscible, and
wherein the Gibbs energy of mixing (DGₘᵢₓ/RT) for the peptide solvent and the peptide non-solvent is less than or equal to about-0.6.
13. The method of paragraph 12, wherein during step (ii), the peptide is precipitated to produce a composition comprising micronized nanoparticles of the peptide, and wherein the micronized nanoparticles have a number average size of 5 microns or less, 1 micron or less, or 300 nm or less.
14. The method of paragraph 12 or 13, wherein the peptide solvent is methanol or water, or a combination thereof, and/or wherein the peptide non-solvent is selected from the group consisting of tert-butanol, 2-propanol, acetonitrile, acetone, ethyl acetate, tetrahydrofuran, dichloromethane, and chloroform, or a combination thereof.
15. The method of any one of paragraphs 12-14, wherein the peptide is a glucagon-like peptide-1 receptor agonist (“GLP-1 RA”).
16. A method for forming nanoparticles comprising a solid, micronized peptide, comprising:
(a) dissolving a polymer in a first suspension to form a second suspension, wherein the first suspension comprises the solid micronized peptide and a polymer solvent, and
(b) introducing the second suspension into a polymer non-solvent to spontaneously form the nanoparticles,
wherein the polymer solvent and the polymer non-solvent are miscible, and
wherein the Gibbs energy of mixing (ΔGₘᵢₓ/RT) for the polymer solvent and the polymer non-solvent is less than or equal to about-0.6.
17. The method of paragraph 16, wherein the method further comprises, prior to step (a),
(i) micronizing a peptide to form the first suspension comprising the micronized peptide.
18. The method of paragraph 16 or 17, wherein step (b) does not include emulsification, agitation, and/or stirring.
19. The method of any one of paragraphs 16-18, wherein the polymer solvent is dichloromethane or chloroform, or a combination thereof, and/or wherein the polymer non-solvent is 2-propanol or heptane, or a combination thereof.
20. Polymeric nanoparticles comprising a micronized peptide encapsulated or dispersed therein, wherein the nanoparticles provide sustained release of the peptide with less than 20% of the peptide released initially (0 hour) following placement into a phosphate buffered saline at 37° C. and room pressure (i.e., 1 atm).
21. The polymeric nanoparticles of paragraph 20, wherein the peptide has a molecular weight of up to 6,000 Da.
22. The polymeric nanoparticles of paragraph 20 or 21, wherein less than 50% of the peptide is released at 24 hours following placement into the phosphate buffered saline.
23. The polymeric nanoparticles of paragraph 20 or 21, wherein less than 50% of the peptide is released at 200 hours following placement into the phosphate buffered saline.
24. The polymeric nanoparticles of paragraph 20 or 21, wherein >50% of the peptide is released at or after 400 hours following placement into the phosphate buffered saline.
25. The polymeric nanoparticles of any one of paragraphs 20-24, wherein the nanoparticles have a number average size of 300 nm or less.
26. The polymeric nanoparticles of any one of paragraphs 20-25, wherein the polymer is a biodegradable polymer, such as a polymer selected from the group consisting of biodegradable polyesters (e.g., polyhydroxyesters), polyanhydrides, poly(lactic acid), poly(glycolic acid), and poly(lactic-co-glycolic acid), or a copolymer thereof, or a mixture thereof.
27. The polymeric nanoparticles of any one of paragraphs 20-26, wherein the polymer is PLGA.
28. The polymeric nanoparticles of paragraph 27, wherein the PLGA has a molecular weight in a range from about 1 kDa to about 20 kDa, from about 1 kDa to about 3 kDa, from about 4 kDa to about 15 kDa, or from about 7 kDa to about 17 kDa.
29. The polymeric nanoparticles of paragraph 27 or 28, wherein the weight ratio of lactic acid to glycolic acid in the PLGA is in a range from 25:75 to 75:25, such as 50:50 or 75:25.
30. The polymeric nanoparticles of any one of paragraphs 20-29, wherein the peptide is a glucagon-like peptide-1 receptor agonist (“GLP-1 RA”).

### EXAMPLES

### Example 1. PLGA Nanoparticles for Long Acting Delivery of a GLP-1 Receptor Agonist Peptide in the Treatment of Type 2 Diabetes

For example, solvent and non-solvent pair with a ΔG_{Mix}/RT value of −1.293 (dichloromethane and heptane) yielded PLGA particles with a number average size of 260 nm while those with a less negative ΔG_{Mix}/RT value of −0.205 (acetone and heptane) yielded particles with a larger number average size of 638 nm. The ΔG_{Mix} of solvent and non-solvent pairs used in the phase inversion process correlates to the GLP-1 RA encapsulation efficiency and release behavior of the drug from PLGA particles. GLP-1 RA encapsulated using solvent and non-solvent pairs with ΔG_{Mix}/RT values (between approximately −1.4 and −0.6) displayed low burst release (0-20% of the theoretical loading at time 0) and released the majority of drug after approximate an 8-day (200 hour) lag period. On the other hand, GLP-1 RA encapsulation using solvent and non-solvent pairs with less negatives ΔG_{Mix}/RT values (between approximately −0.6 and −0.2) displayed a large burst release (approximately 20-70% of the theoretical loading at time 0), with the majority of the drug being released by the 1-day (24 hour) time point (approximately 60-100% of the theoretical loading).

### Materials and Methods

### Preparation of Micronized GLP-1 RA, PLGA Nanoparticles, and GLP-1 RA Loaded PLGA Nanoparticles

All nanoparticles were prepared by phase inversion as described previously (Mathiowitz, E. Adv. Drug Deliv. Rev. 65, 811-821 (2013); Jacob, J. S. & Mathiowitz, E. Carr. Based Drug Deliv. 214-223 (2004). doi: 10.1021/bk-2004-0879.ch015). The phase inversion nanoencapsulation process includes the steps of first micronizing the drug and then encapsulating in a polymer nanoparticle. Specifically, the drug is micronized by solubilizing in a good solvent and then adding the solution to a reservoir of non-solvent for the drug. The solvent and non-solvent are miscible with each other, which allows for the drug solvent to be extracted into the non-solvent and thus result in drug precipitation. Upon extraction of the solvent by the miscible non-solvent, the drug is believed to precipitate through the steps of supersaturation, nucleation and growth. The resulting product is micronized drug nanoparticles.

Here, micronization and encapsulation of a single peptide drug (a GLP-1 receptor agonist peptide used to treat type 2 diabetes, provided by Sanofi S.A. (referred to as “Sanofi drug”)) is exemplified. Various solvent and non-solvent combinations were analyzed during the GLP-1 RA micronization process. Both methanol and water were analyzed as peptide solvents to solubilize the GLP-1 RA. In all instances the peptide was dissolved as a concentration of 20 mg/mL. From these solvents, the GLP-1 RA was precipitated into different peptide non-solvents: tert-butanol, 2-propanol, acetonitrile, acetone, ethyl acetate, tetrahydrofuran, dichloromethane, and chloroform. In all instances, the ratio of solvent to non-solvent was 1 mL: 60 mL, in order to ensure reservoir conditions for the extraction of solvent.

Phase inversion of pure PLGA particles (i.e., blank particles, not containing drug) was studied to better understand the phase inversion precipitation process without the peptide. In the analysis of blank PLGA particle formation, PLGA (75:25) with Mw=4-15 kDa was used (Resomer RG 752H, CAS 26780-50-7). The following polymer solvents were tested in the phase inversion process: dichloromethane, ethyl acetate, chloroform, acetone, acetonitrile, and tetrahydrofuran. The following polymer non-solvents were tested: tert-butanol, 2-propanol, ethanol, heptane, and water. In all instances, the polymer was solubilized in each solvent at a concentration of 100 mg/mL. The solubilized polymer solution was then added to an excess of non-solvent to facilitate precipitation and particle formation. The ratio of solvent to non-solvent was 1 mL: 60 mL, to ensure reservoir conditions for the extraction of solvent.

In the study of GLP-1 RA encapsulation and release from PLGA nanoparticles, three types of PLGA were used (PLGA (50:50) with Mw=7-17 kDa, Resomer 502H (CAS Number 26780-50-7), PLGA (75:25) with Mw=4-15 kDa Resomer 752H (CAS Number 26780-50-7), and PLGA (50:50) with Mw 2.3 kDa, Evonik. GLP-1 RA was micronized using methanol as the peptide solvent in all instances. Upon solubilizing the drug in methanol, the solution was added to different peptide non-solvents to create the micronized drug product (non-solvents: dichloromethane, chloroform, ethyl acetate, tetrahydrofuran, acetonitrile, and acetone). In each instance of micronization, the peptide was solubilized at a concentration of 20 mg/mL and precipitated with a peptide solvent: peptide non-solvent ratio of 1 mL: 60 mL. Upon precipitating the GLP-1 RA peptide in various peptide non-solvents, the PLGA was then solubilized in each of the suspended micronized drug solution. The GLP-1 RA was micronized with peptide non-solvents of dichloromethane, chloroform, ethyl acetate, tetrahydrofuran, acetone, and acetonitrile. While these organic solvents are not solvents for the peptide, they are solvents for the polymer. Thus, in the cases where the PIN process uses both polymers and peptides to form polymeric nanoparticles encapsulating peptides, the polymer solvent is denoted as PLGA solvent or GLP-1 RA non-solvent and the polymer non-solvent is denoted as PLGA non-solvent, which is also a GLP-1 RA non-solvent (see, e.g., denotations in Table 5 below). In this exemplary case of PLGA nanoparticles encapsulating GLP-1 RA, the peptide non-solvent serves as the polymer solvent, such that PLGA is dissolved in the peptide non-solvent of the solution of micronized GLP-A RA. Thus, the PLGA was added to each micronized drug solution at a concentration of 100 mg/mL to produce a solution of solubilized polymer and suspended micronized drug. The final step in the phase inversion nanoencapsulation process was to add each solution to a non-solvent for both the polymer and drug. 2-propanol and heptane were used as non-solvents for the final nanoparticle products. Upon phase inversion and precipitation, the nanoparticles were run through a positive pressure filtration column with a PTFE filter to collect the resulting nanoparticles and then lyophilized to remove residual solvents. An advantage of this process is that separation of the micronized peptide after the first micritization process is avoided and thus making the process easier to scale up, compared to previous PIN process, such as that described in U.S. Publication No. 2010/0172998.

### Interaction Parameter Calculations

Upon immersing the polymer/drug solution to a non-solvent, there is a complex web of interactions that has an impact on the resulting product. A complete understanding of this process would require consideration of not only solvent/non-solvent interactions, but also polymer/solvent, polymer/non-solvent, drug/solvent, drug/non-solvent, and polymer/drug interactions. For the bulk of this analysis, ΔG_{Mix} between these components was assumed to be negligible. This assumption is justified in part by the utilization of dilute polymer/drug solutions, which reduced their contribution to the total ΔG_{Mix}.

However, in some instances, the affinity of the polymer to the solvent was considered using the Flory Huggins interaction parameter-dimensionless parameter X₁₂, which can be approximated based on the Hildebrand solubility parameter:

$${X}_{12} \approx \frac{{v}_{1}}{RT} {( {δ}_{2} - {δ}_{2} )}^{2}$$

where v₁ is the molar volume of the solvent and 81 and 82 are the solubility parameters of solvent and polymer respectively. The Flory Huggins interaction parameter accounts for the energies between polymer-polymer, polymer-solvent, and solvent-solvent. This equation is not regarded as being accurate for quantitative measurements, but rather serves as a qualitative guide when considering polymer solubility. The general notation is such that low X₁₂ values indicate good solubility.

### Size Measurements

The size of both the micronized GLP-1RA and blank PLGA NPs were determined using a Zetasizer Nano ZS Size Analyzer from Malvern Panalytical. Suspensions of 1 mg/mL were produced for all GLP-1 RA and PLGA NPs by brief vortex and sonication. Milli-Q water was used as a dispersant for PLGA and acetonitrile was used as a dispersant for GLP-1RA. Each suspension was placed in a quartz cuvette for size analysis. Scan were conducted in triplicates on each sample and the average particle sizes from each scan were compared.

### Scanning Electron Microscopy

Particles were imaged with a ThermoScientifc Apreo VS SEM. Sample were prepared by applying the particles to a carbon-adhesive tab, mounted on an aluminum stub and sputter-coated with Au-Pd using a Polaron Sputter Coater. Samples were examined at 3 kV accelerating voltage.

### Fourier Transform Infrared Spectroscopy

Following lyophilization of the micronized drug, 1.0±0.05 mg of each micronization was combined with 99.0±0.05 mg of FTIR-grade KBr and pressed into a pellet. First a background scan was conducted, and absorption spectra were recorded over 32 scans from 400-4000 cm −1 in 1 cm −1 steps. Using Spectragryph software, each spectrum was baseline corrected and normalized using the amide peak at 1650 cm −1. FITYK software was then used for the deconvolution and analysis.

### In-Vitro Release Studies

In-vitro drug release profiles were assessed by placing approximately 20 mg of each nanoparticle formulation into a 1.5 mL eppendorf tube containing phosphate buffered saline (PBS). At varying times points, the nanoparticles were spun down in a microcentrifuge at 8000 rpm for 10 minutes and the supernatant was collected for HPLC analysis. The nanoparticles were then resuspended in fresh PBS by brief vortex and sonication. Samples obtained from the release study were analyzed against a standard curve by reverse phase chromatography (using a Phenomenex, Aeris 3.6 μm Widepore XB-C18 column). A Waters 600 controller was used to maintain a flow rate of 0.5 mL/min of an acetonitrile and water mobile phase gradient containing 0.1% formic acid. A UV detection wavelength of 215 nm was used to obtain the chromatograms.

### Results and Discussion

### GLP-1 RA Micronization and Stability

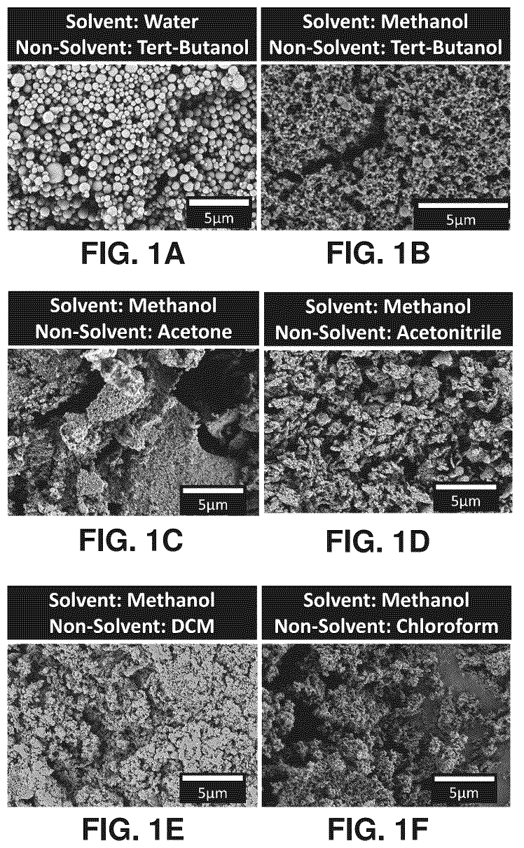

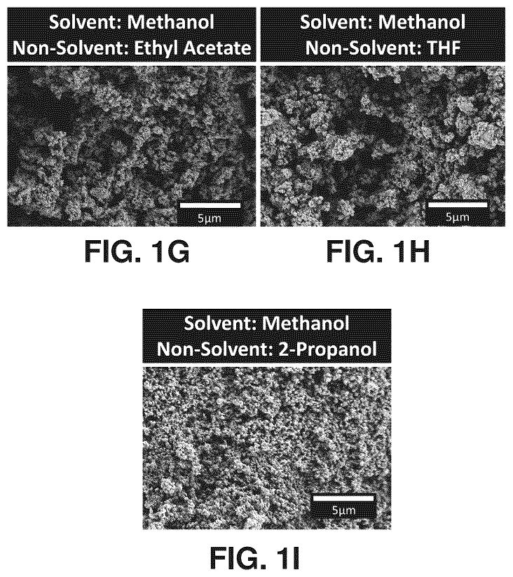

Micronization of the GLP-1 RA were performed under the conditions described in Table 2. The resulting GLP-1 RA particle diameter was determined using a Malvern Zetasizer. Corresponding SEM images of each GLP-1 RA micronization are shown in FIGS. 1A-1I. Briefly, the GLP-1 RA was solubilized in either water or methanol at a concentration of 20 mg/mL and then precipitated into one of eight different non-solvents to produce a micronized particle (non-solvents: tert-butanol, 2-propanol, dichloromethane, chloroform, ethyl acetate, tetrahydrofuran, acetone, and acetonitrile). From the SEM images, each micronization process resulted in the formation of discrete spherical nanoparticles. As demonstrated by the SEM images (FIGS. 1A-1I), the drug particles are smaller than 5 microns, such as smaller than 4 microns, smaller than 3 microns, smaller than 2 microns, or smaller than 1 micron. Particle size analysis showed that the particles produced with methanol as the peptide solvent have a number average particle size (also referred to herein as “average particle diameter”) between 302-537 nm, while the particles produced with water as the peptide solvent were substantially larger with an average diameter of 1304 nm (Table 2). These are consistent with the SEM images (FIGS. 1A-1I).

TABLE 2
GLP-1 RA micronization conditions with the corresponding Gibbs energy of mixing
between the solvent/non-solvent and the resulting micronized drug particle size

| | | | | Average | |
|---|---|---|---|---|---|
| Drug | Concentration | Solvent | Non-Solvent | Size (nm) | ΔG_{Mix} /RT |
| GLP-1 RA | 20 mg/mL | Water | TBA | 1304 | −0.333 |
| peptide | | Methanol | TBA | 418 | −0.658 |
| | | | 2-Propanol | 324 | −0.97 |
| | | | Acetonitrile | 302 | −0.729 |
| | | | Acetone | 537 | −0.443 |
| | | | Dichloromethane | 460 | −0.506 |
| | | | Chloroform | 414 | −0.553 |
| | | | Ethyl Acetate | 404 | −0.486 |
| | | | THF | 381 | — |

The micronization of GLP-1 RA was to understand the effect of varying solvent and non-solvent pairs on the formation of drug particles upon phase inversion. The solvents and non-solvents used for the micronization of the GLP-1 RA were miscible in all proportions.

Without being bound to any theories, when two miscible fluids come into contact, there is an initial sharp interface in which diffusion of one liquid into the other occurs. Over time the fluids eventually mix creating a homogenous solution. Upon mixing, the precipitation of micronized drug particles is thought to occur in three general steps: supersaturation, nucleation, and growth. Supersaturation occurs when the solution contains more dissolved solute than is thermodynamically stable. Thus, to gain thermodynamic stability, the solute begins to precipitate by nucleation and growth. Based on this mechanism, the rate at which the solvent and non-solvent mix is believed to impact the degree of supersaturation, nucleation, and growth of particles in the phase inversion process. For example, if the diffusion of solvent to non-solvent is ‘fast’ then a high degree of supersaturation is expected, resulting in more nucleation centers, less growth, and the formation of smaller nanoparticles. On the other hand, if the diffusion of solvent to non-solvent is ‘slow’ then the GLP-1 RA peptide is expected to reach a lower degree of supersaturation, allowing for less nucleation and larger particles to form.

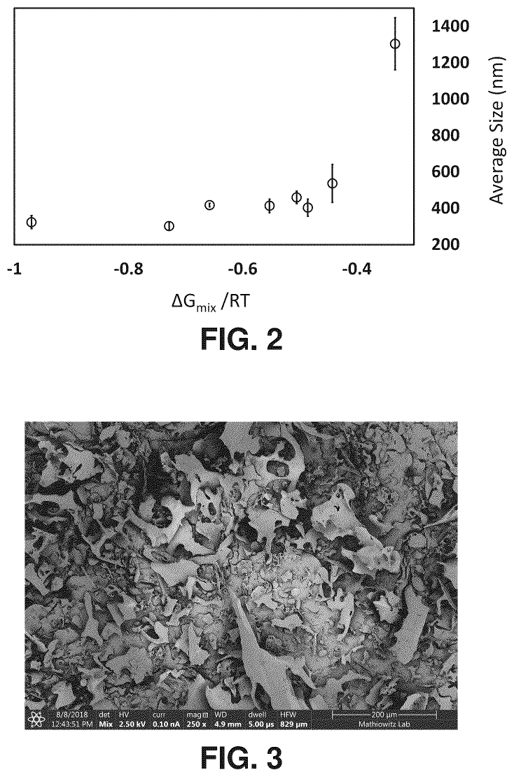

The semi-empirical UNIFAC method was adopted here for modeling non-ideal liquid mixtures as a means of describing the interactions between the solvents and non-solvents used for particle formation. Specifically, the UNIFAC interaction parameters were used to calculate the Gibbs energy of mixing (ΔG_{Mix}) for each solvent and non-solvent pair. ΔG_{Mix} was used because it correlates to the rate at which the solvent diffuses into the non-solvent. A negative ΔG_{Mix} indicates favorable diffusion between the solvent and non-solvent. Accordingly, if ΔG_{Mix} is positive, the solvent and non-solvent will phase separate and a homogenous mixture will not spontaneously form. Thus, a comparative analysis of nanoparticle size and Gibbs energy of mixing was conducted (FIG. 2). As demonstrated in FIG. 2, use of the method described herein allows one to select and control particle size by selecting solvent-non solvent pairs and calculating ΔG_{Mix} for a given set of conditions (e.g. same temperature, same mixing conditions, or no mixing). For example, if the desired size of the micronized GLP-1RA peptide is <550 nm, then ΔGₘᵢₓ/RT should be more negative than-0.333. Exemplary solvent-non-solvent pairs suitable for achieving such a ΔGₘᵢₓ/RT and thus micronized GLP-1RA peptide <550 nm include, but are not limited to, tert-butanol-methanol, 2-propanol-methanol, acetonitrile-methanol, acetone-methanol, dicholormethane-methanol, chloroform-methanol, ethyl acetate-methanol, and tetrahydrofuran-methanol.

### Micronized Drug Particle Size

FIG. 3 shows an SEM image of the GLP-1 RA peptide as it was received from Sanofi (i.e. prior to micronization). Based on the SEM image, the peptide is in the form of a lyophilized white powder, with large aggregates and sheets on the order of 50-200 μm. The first goal was to achieve bioactive micronization of the GLP-1 RA peptide. Achieving sufficiently small micronization during the PIN process affects the encapsulation and subsequent long-acting controlled release of the drug from phase inverted PLGA nanoparticles. When sufficiently small, the solid micronized GLP-1 RA particles are expected to act as a core nucleus in which the polymer in solution will precipitate around during the PIN process.

The Gibbs energy of mixing calculated by the UNIFAC method is shown in Table 2 for all solvent and non-solvent pairs used in the GLP-1 RA micronization process. FIG. 2 demonstrates the relationship between ΔG_{Mix} and the resulting GLP-1 RA particle size. Generally, the largest particles (average diameter=1304 nm) were formed from water and tert-butanol mixing, which also had the least negative ΔG_{Mix} (ΔG_{Mix}/RT=−0.333). All other solvent and non-solvents with increasingly negative ΔG_{Mix} (those produced from methanol as a solvent with ΔG_{Mix}/RT between −0.443 and −0.970) did not show a significant correlation with size (average diameters between 302 nm and 537 nm). This indicates that the GLP-1 RA particles formed may have reached a minimum critical size that is stable against dissolution.

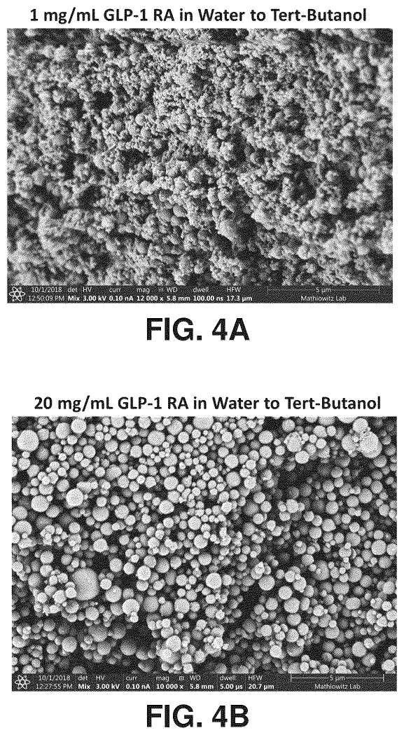

The concentration of the solute in solvent is also a factor affecting the size of the precipitated nanoparticles (Jacob, J. S. & Mathiowitz, E. A Novel Mechanism for Spontaneous Encapsulation of Active Agents: Phase Inversion Nanoencapsulation. Carr. Based Drug Deliv. 214-223 (2004). doi: 10.1021/bk-2004-0879.ch015). This is demonstrated in FIGS. 4A and 4B with the GLP-1 RA peptide precipitated from water to tert-butanol. In each instance of FIGS. 4A and 4B, the GLP-1 RA was solubilized in water at either a concentration of 1 mg/mL (left) or 20 mg/mL (right). The peptide was then precipitated by immersing the soluble solution to a non-solvent (tert-butanol) with a solvent to non-solvent ratio of 1 mL: 60 mL. As shown in FIGS. 4A and 4B, lower concentrations of drug in the solvent (1 mg/mL compared to 20 mg/mL) yield smaller particles upon phase inversion. Without being bound to any theories, upon nucleation, particles are thought to grow as a result of condensation, in which the addition of single molecules collect on the nucleation surface to form a particle. The ability for this growth to occur appears to directly relate to the concentration of drug in proximity to the primary nuclei. The trend shown in FIG. 2 may also indicate that nanoparticles converge on a minimum size based on the constant 20 mg/mL concentration used, regardless of the ΔG_{Mix} between the solvent and non-solvent.

### GLP-1 RA Stability Upon Micronization

The feasibility of the GLP-1 RA peptide to retain its native bioactive structure upon micronization was assessed. The GLP-1 RA peptide has a well-defined and unique folded state, in which the tertiary structure attributes to the native bioactive form. Typically, the unfolding of this state is considered to denature the peptide and result in the loss of therapeutic bioactivity. The use of organic solvents (such as those used for drug micronization in Table 1) have conventionally been thought to denature peptides from their native bioactive state. The feasibility of retaining the native GLP-1 RA state upon micronization with organic solvents as described above was explored.

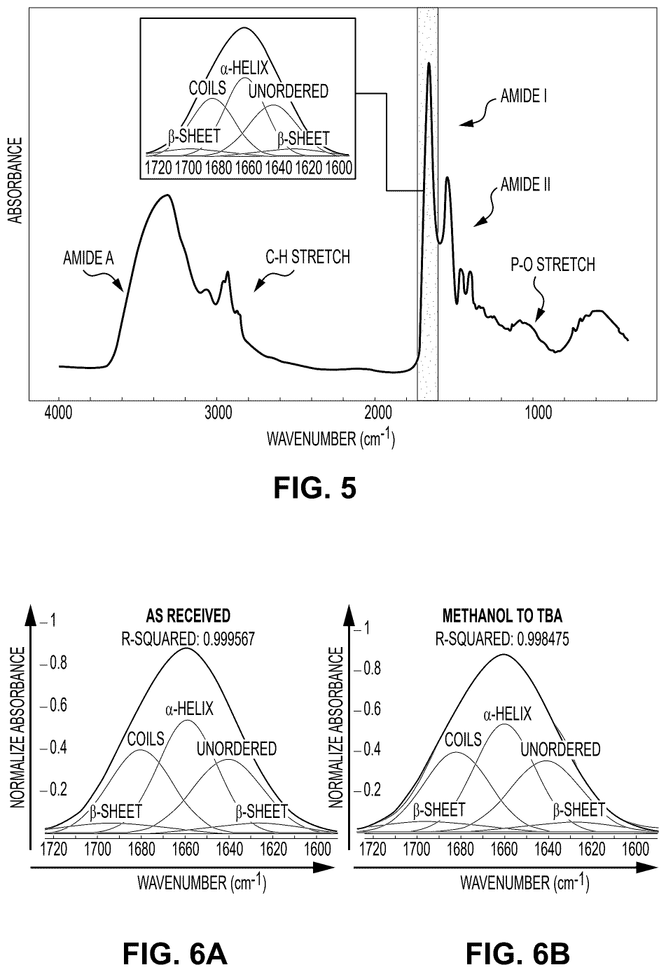

FTIR spectroscopy was used to provide data showing the GLP-1 RA conformation and stability upon micronization with various organic solvents. The Amide I band is sensitive to protein secondary structure, with shifts in the frequency indicating structural change. In addition, due to the unique hydrogen bonding environments of the coil, a-helix, β-sheet, and amorphous secondary structures, the corresponding contributions can be approximated by peak deconvolution. FIG. 5 shows the full FTIR spectrograph of the GLP-1 RA as it was received from Sanofi. A peak deconvolution was performed on the Amide I band of the as-received GLP-1 RA FTIR spectrograph to achieve a curve fitting correlation of R²>0.99. The deconvolution assignments of coils, a-helix, β-sheet, and amorphous secondary structures were based on previous reports (Jackson, M. & Mantsch, H. H. The Use and Misuse of FTIR Spectroscopy in the Determination of Protein Structure. Crit. Rev. Biochem. Mol. Biol. 30, 95-120 (1995)). The peak deconvolution obtained from the as-received GLP-1 RA FTIR was then applied to the Amide I FTIR spectra of each micronized GLP-1 RA that were subjected to organic solvent environments (see, e.g., the representative spectra shown in FIGS. 6A-6B). Surprisingly, using an identical deconvolution profile, no significant shifts or changes in secondary structure conformation was detected in the micronized drug, with each curve fitting correlation demonstrating an R² value greater than 0.99.

HPLC was used to compare the conformation of the micronized GLP-1 RA peptide to the drug as it was received from Sanofi, in order to give further insight to the stability of the peptide during the micronization process. Upon denaturation by organic solvent, peptide residues that are typically buried in the core of the folded native structure are thought to become exposed via unfolding and interact with the external solvent. As a result of this unfolding, denaturation typically results in the peptide occupying a larger volume compared to the compact native state. In addition, denaturation is expected to change the hydrophilic/hydrophobic nature of the peptide, as hydrophobic residues that were once buried become exposed to the solvent. The HPLC method here utilized a reverse phase Phenomenex, Aeris 3.6 μm Widepore XB-C18 (100×2.1 mm) column, which contained a hydrophobic stationary phase and allowed for peptide separation based on size and charge.

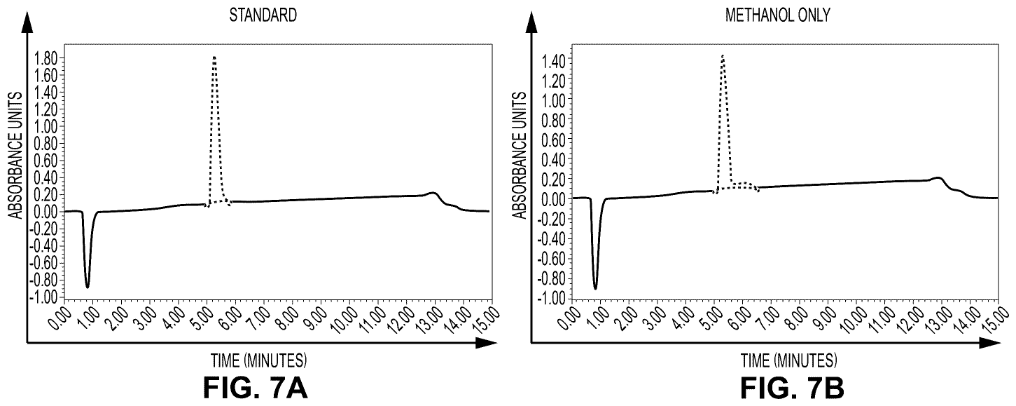

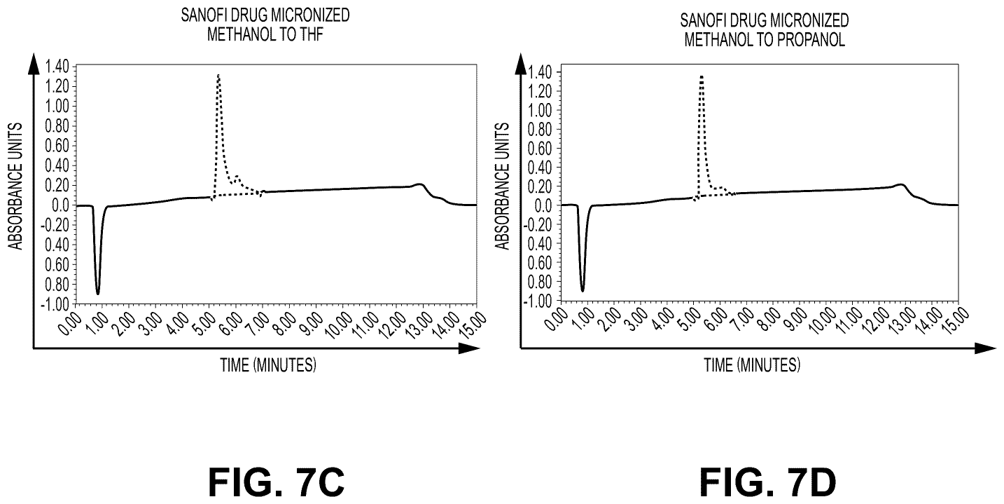

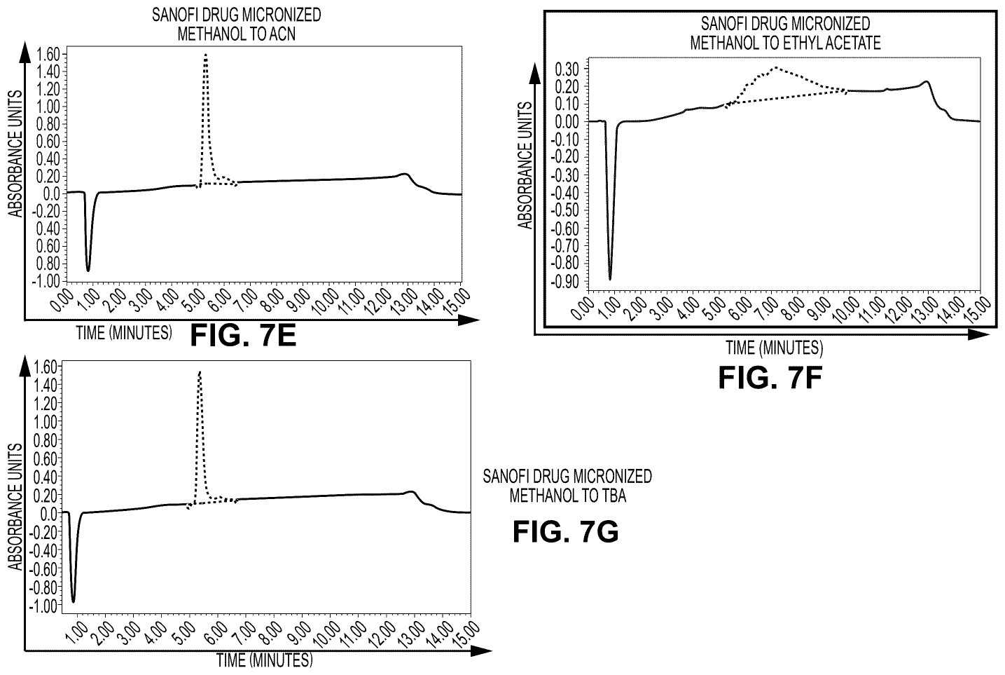

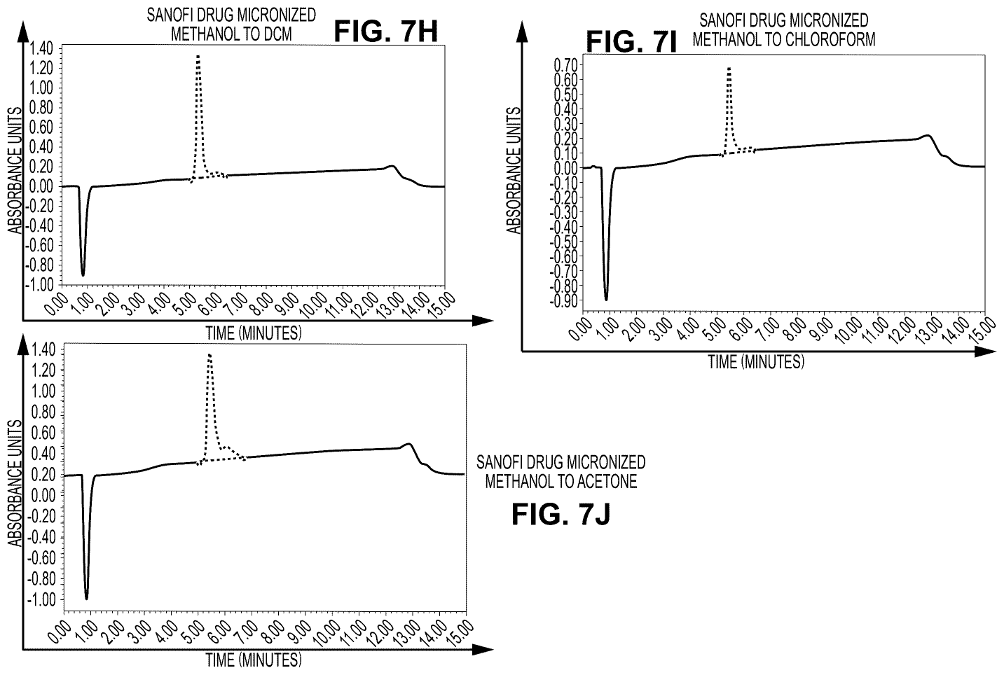

HPLC chromatograms of eluted GLP-1 RA are shown in FIGS. 7A-7J (where the x-axis is time (minutes) and the y-axis is (absorbance units)). These HPLC chromatograms demonstrate changes in the chromatogram peak shape of different micronization solvent-non-solvent pairs. The qualified retention time of each micronization shown in FIGS. 7A-7J is provided in Table 3. Table 3 shows that the as received GLP-1 RA peptide had a retention time of 5.354 min, while micronization using different solvent-non-solvent pairs had retention times ranging from 5.303 min to 5.446 min. The similarity of the retention times between the as received GLP-1 RA provides and micronized GLP-1 RA further supports that the drug retained the native state upon micronization, with no significant change in hydrophobicity or size.

While retention time was predominantly similar between the samples, FIGS. 7A-7J show that some micronizations resulted in the formation of a small secondary peak around a 6 min retention time. This secondary peak is most significant in the micronization with ethyl acetate, tetrahydrofuran, or acetone as the non-solvent (See, e.g., FIGS. 7F, 7C, 7J, respectively). The formation of this peak indicates that some of the drug has undergone a conformational change with a variation in hydrophobicity or size compared to the native as received GLP-1 RA. As the retention time is longer (˜6 min) compared to the native state (˜5.3 min), these results show that the conformational change is more hydrophobic with a greater interaction to the hydrophobic stationary phase of the reverse phase HPLC column. When looking at the chemical structure of the organic solvents used during micronization, ethyl acetate, tetrahydrofuran, and acetone are all aprotic solvents and contain highly electronegative oxygen species which can act as hydrogen bond ‘acceptors’. Thus it may be that aprotic organic solvents can disrupt the intra-molecular bonds that stabilize the GLP-1 RA tertiary structure, resulting in buried hydrophobic residues being exposed to the surface. While in some micronization steps, the particular peptide non-solvent appeared to alter the native conformation in a portion of the GLP-1 RA, the majority of the drug maintained the same size and hydrophobicity across all micronization conditions as demonstrated by the FTIR spectra and HPLC chromatograms (see, e.g., FIGS. 6A-6B and 7A-7J).

TABLE 3
Reverse Phase HPLC retention times of GLP-1 RA micronized
from various solvent and non-solvent combinations

| | Solvent | Non-solvent | Retention Time |
|---|---|---|---|
| As Received | | | 5.354 |
| Methanol Only | | | 5.316 |
| | Methanol | Tert-Butanol | 5.331 |
| | | Dichloromethane | 5.335 |
| | | Chloroform | 5.396 |
| | | Ethyl Acetate | 5.399 |
| | | Tetrahydrofuran | 5.341 |
| | | Acetone | 5.446 |
| | | Acetonitrile | 5.303 |
| | | 2-Propanol | 5.311 |
| | Water | Tert-Butanol | 5.331 |

### PLGA Nanoparticle Formation by Phase Inversion

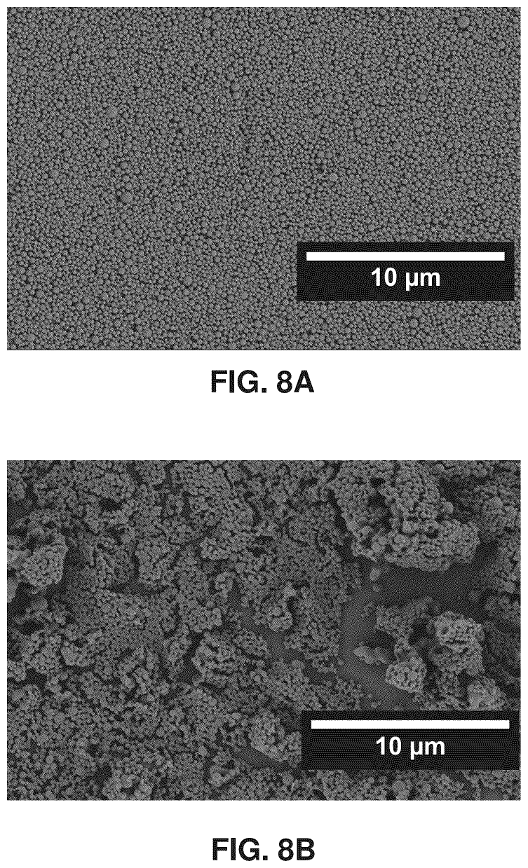

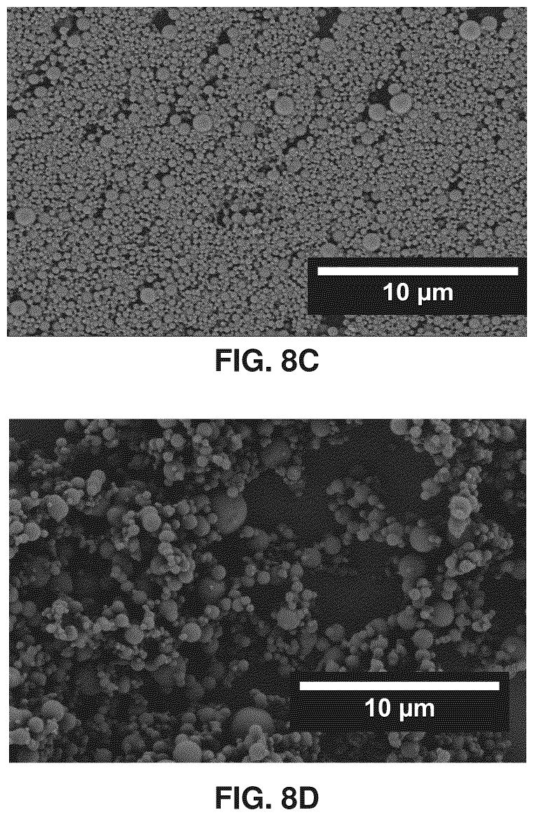

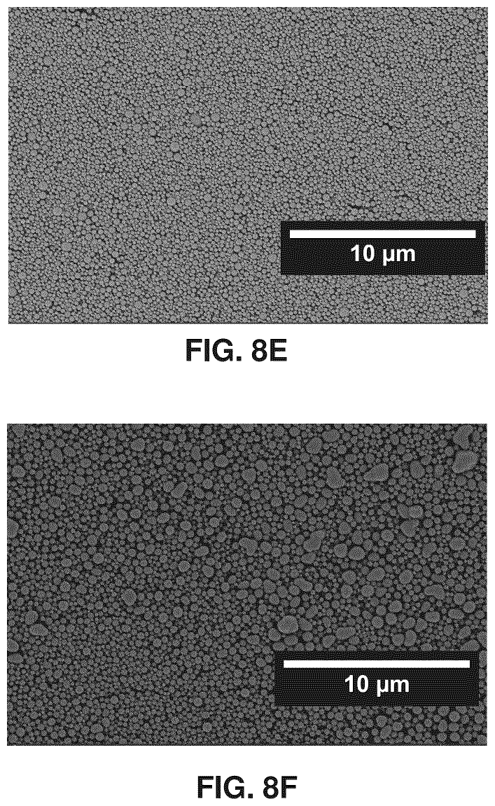

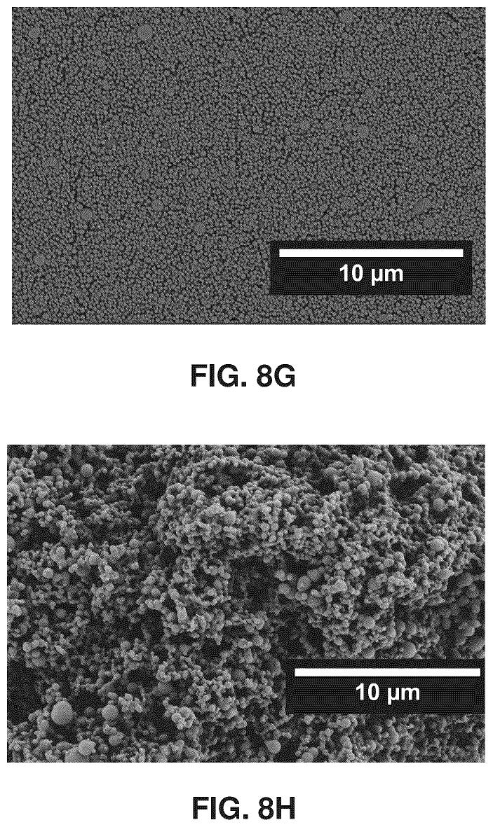

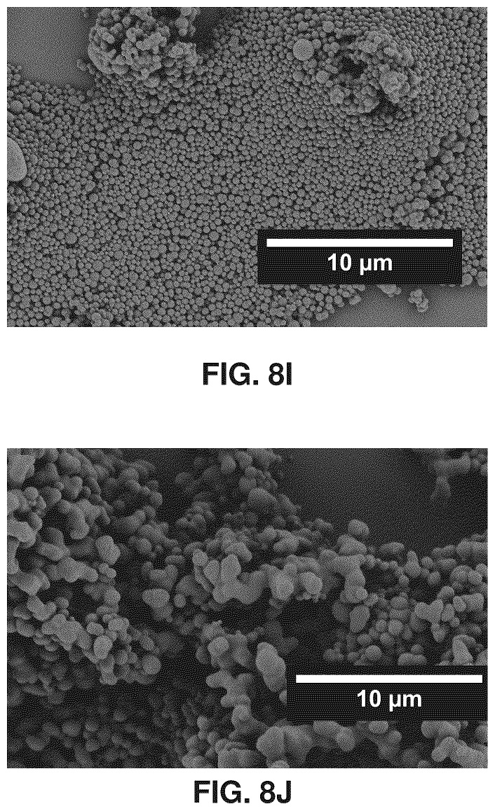

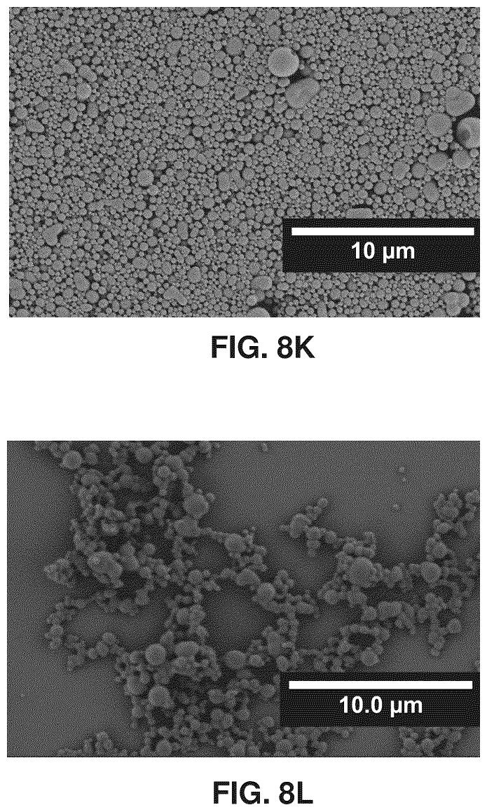

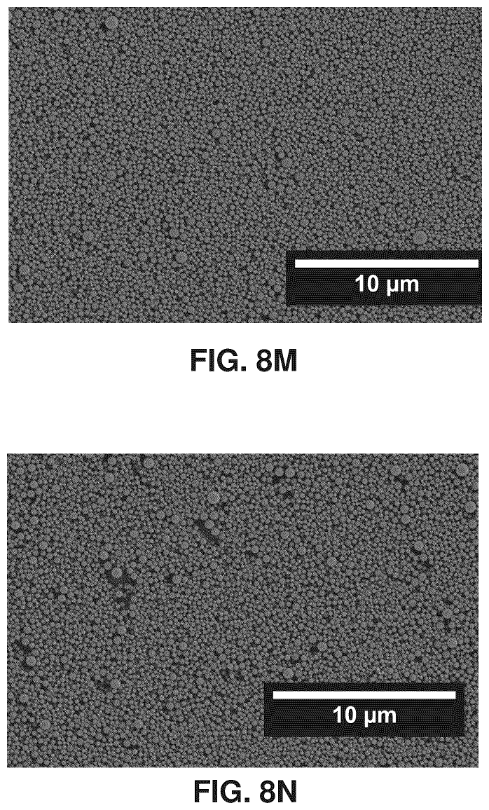

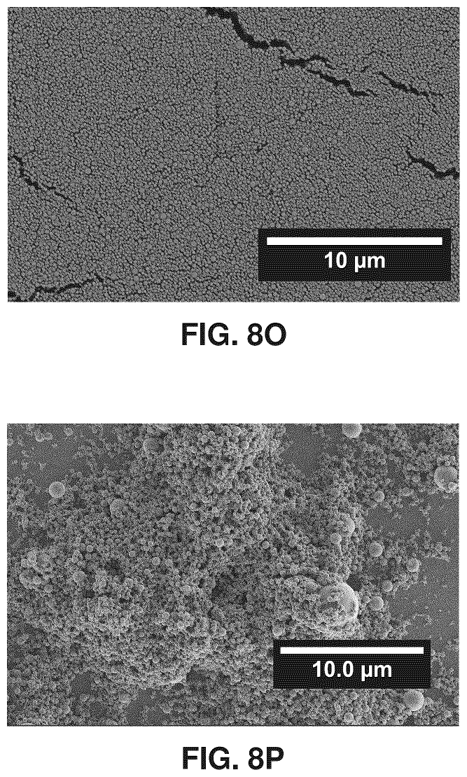

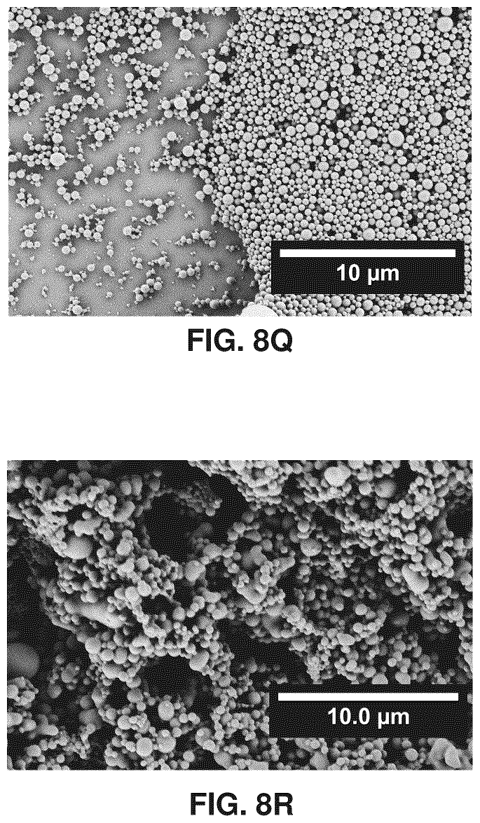

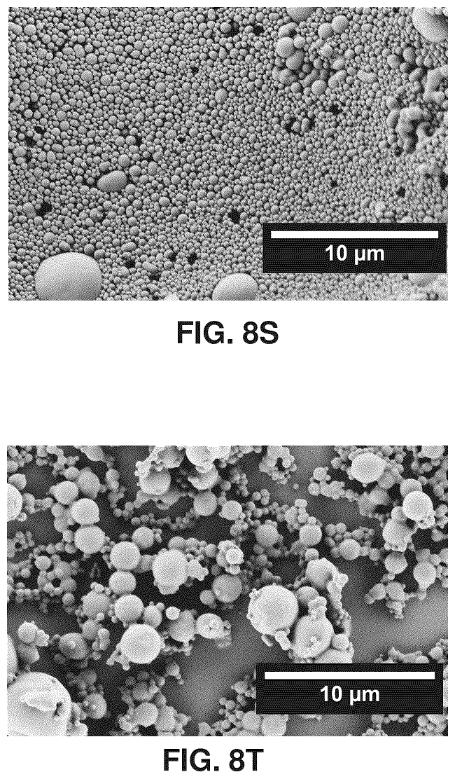

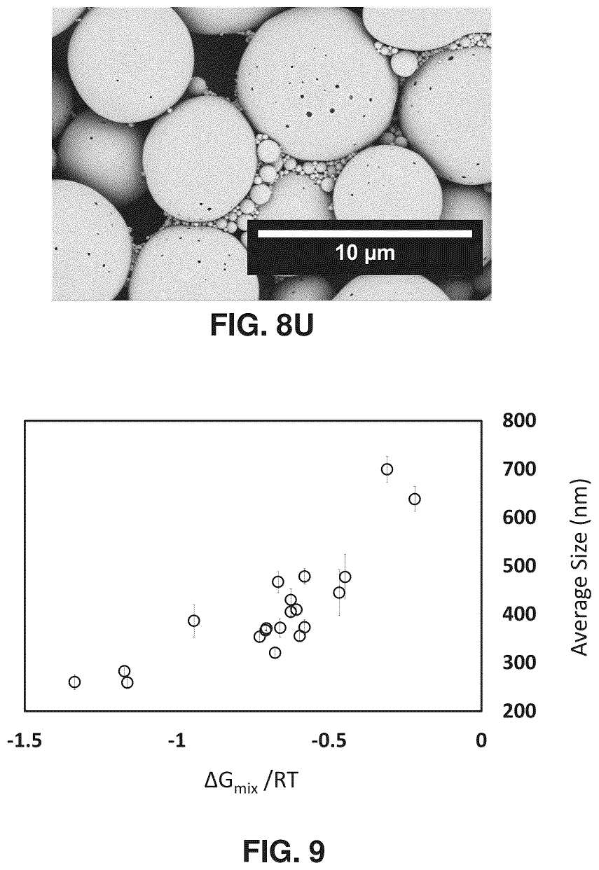

To better understand the phase inversion precipitation process, the impact of solvent and non-solvent on the formation of pure PLGA nanoparticles by phase inversion (i.e., blank nanoparticles, not containing drug) was studied. 4-15 kDa PLGA (75:25) was used as an example. PLGA particles were produced by phase inversion under the conditions described in Table 4 and the resulting particle diameter was determined using a Malvern Zetasizer. In addition, corresponding SEM images of each PLGA nanoparticle formed are shown in FIGS. 8A-8U. As shown in the SEM images, each precipitation process resulted in the formation of discrete particles. The images shown in FIGS. 8A-8U provide a qualitative assessment of the particle size and morphology that supports the size quantified by the Malvern Zetasizer shown in Table 4.

TABLE 4
Conditions used to produce PLGA nanoparticles with corresponding
Gibbs energy of mixing between each solvent pair and the
resulting average size of the formed nanoparticles

| | | | | Average | |
|---|---|---|---|---|---|
| Polymer | Concentration | Solvent | Non-Solvent | Size (nm) | ΔG_{Mix} /RT |
| Resomer ® | 100 mg/mL | Dichloromethane | Tert-Butanol | 386.87 | −0.929 |
| RG 752 H, | | | 2-Propanol | 282.93 | −1.161 |
| PLGA (75:25) | | | Ethanol | 367.1 | −0.71 |
| 4-15 kDa | | | Heptane | 260.17 | −1.293 |
| | | Chloroform | Tert-Butanol | 467.47 | −0.666 |
| | | | 2-Propanol | 259.3 | −1.164 |
| | | | Ethanol | 430.1 | −0.62 |
| | | | Heptane | 320.6 | −0.63 |
| | | Ethyl Acetate | Tert-Butanol | 372.17 | −0.661 |
| | | | 2-Propanol | 699.87 | −0.297 |
| | | | Ethanol | 478.97 | −0.565 |
| | | | Heptane | 373.43 | −0.593 |
| | | Acetone | Tert-Butanol | 477.8 | −0.441 |
| | | | 2-Propanol | 405.03 | −0.627 |
| | | | Ethanol | 445 | −0.464 |
| | | | Heptane | 638.53 | −0.205 |
| | | | Water | 5201.67 | −0.128 |
| | | Acetonitrile | Tert-Butanol | 409.57 | −0.539 |
| | | | 2-Propanol | 353.7 | −0.712 |
| | | | Ethanol | 370.8 | −0.701 |
| | | | Water | 355.5 | −0.473 |

FIG. 9 shows the correlation of ΔG_{Mix} to the resulting PLGA nanoparticle size. ΔG_{Mix} is indicative of the rate at which the solvent and non-solvent mix to create a homogenous solution, impacting the degree of supersaturation, nucleation, and growth of the PLGA nanoparticles upon phase inversion. As shown in FIG. 9, there is a general correlation between ΔG_{Mix} and PLGA nanoparticle size, with the least negative ΔG_{Mix} resulting in larger particles than those with the most negative ΔG_{Mix}. This correlation further confirms the impact of ΔG_{Mix} that was observed for the GLP-1 RA nanoparticles described above, and demonstrates that Gibbs energy of mixing between solvent and non-solvent is useful for predicting phase inverted nanoparticle size. The information shown in Table 4 and FIG. 9 regarding size measured by Malvern and Gibbs energy is qualitatively supported by the SEM images as shown in FIGS. 8A-8U. For the large particles produced using an acetone-water pair, both a large size (˜5000 nm) was measured by Malvern (Table 4) and large particles were shown by SEM (see, e.g., FIG. 8U). As shown by SEM images, the particles produced by each solvent-non-solvent pair are generally spherical in shape and similar in size. Only FIGS. 8T and 8U show particles that are larger than 5 microns. As shown by the SEM images, all of the particles produced using the various solvent-non-solvent pairs are less than 10 microns, less than 5 microns, or less than 2 microns in diameter.

Further, FIG. 9 demonstrates that use of the method described herein allows one to select and control polymeric particle size by selecting solvent-non solvent pairs and calculating ΔG_{Mix}. For example, if the desired size of the Resomer® RG 752 H, PLGA (75:25) 4-15 kDa is <500 nm, then ΔGₘᵢₓ/RT should be more negative than-0.297. Exemplary solvent-non-solvent pairs suitable for achieving such a ΔGₘᵢₓ/RT include, but are not limited to, dicholormethane-tertbutanol, dichloromethane-2-propanol, dichloromethane-ethanol, dichloromethane-ethanol, dichloromethane-heptane, chloroform-tertbutanol, chloroform-2-propanol, chloroform-ethanol, chloroform-heptane, ethyl acetate-tertbutanol, ethyl acetate-ethanol, ethyl acetate-heptane, acetone-tertbutanol, acetone-2-propanol, acetone-ethanol, acetonitrile-tertbutanol, acetonitrile-2-propanol, acetonitrile-ethanol, and acetonitrile-water.

GLP-1 RA Encapsulation and In-Vitro Release from PLGA Nanoparticles

The effect of solvent/non-solvent choice in the PIN process on the resulting encapsulation and release of GLP-1 RA from PLGA nanoparticles was studied. Table 5 lists all 33 nanoparticle formulations that were produced for the in-vitro release experiments. A fixed drug loading of 5% (w/w) GLP-1 RA in all nanoparticle formulations was used. The variables assed between formulations were (1) PLGA molecular weight and monomer ratio, (2) polymer solvent/drug non-solvent type, and (3) non-solvent type for both the polymer and drug in the encapsulation process.

Three different types of PLGA were assessed in this study (2.3 kDa PLGA (50:50), 4-15 kDa PLGA (50:50), and 7-17 kDa PLGA (75:25)). These polymers were chosen to give insight into the impact of varying molecular weight and monomer ratio on the final product and to determine the robustness of the formulation between these variations.

As examples, several polymer solvents (i.e., peptide non-solvent) types were analyzed (dichloromethane, choloroform, acetonitrile, acetone, ethyl acetate, and tetrahydrofuran). The term “polymer solvent” refers to the organic solvent into which the GLP-1 RA is precipitated, such as one of dichloromethane, choloroform, acetonitrile, acetone, ethyl acetate, and tetrahydrofuran, or a combination thereof. Upon peptide precipitation, the PLGA polymer is added to the suspension containing the micronized peptide. In these instances, the GLP-1 RA is a solid, micronized peptide, while the PLGA is soluble in the polymer solvent. The variations in these organic solvents were chosen to represent a spectrum of polar/nonpolar and protic/aprotic solvents that are compatible with the PIN process.

TABLE 5
GLP-1 RA loaded PLGA nanoparticles formulations produced for in-vitro studies
along with the various solvents and non-solvents used to produce them

| | | Concentration | PLGA Solvent / | PLGA and |
|---|---|---|---|---|
| | GLP-1 RA | of Polymer in | GLP-1 RA | GLP-1 RA |
| PLGA type | Loading | Solvent | Non-Solvent | Non-Solvent |
| 2.3 kDa PLGA | 5% (w/w) | 100 mg/mL | Dichlormethane | 2-Propanol |
| (50:50) Evonik | | | | Heptane |
| | | | Chloroform | 2-Propanol |
| | | | | Heptane |
| | | | Ethyl Acetate | 2-Propanol |
| | | | | Heptane |
| | | | Tetrahydrofuran | 2-Propanol |
| | | | | Heptane |
| | | | Acetone | 2-Propanol |
| | | | | Heptane |
| | | | Acetonitrile | 2-Propanol |
| 7-17 kDa | 5% (w/w) | 100 mg/mL | Dichlormethane | 2-Propanol |
| PLGA (50:50), | | | | Heptane |
| Resomer 502H | | | Chloroform | 2-Propanol |
| | | | | Heptane |
| | | | Ethyl Acetate | 2-Propanol |
| | | | | Heptane |
| | | | Tetrahydrofuran | 2-Propanol |
| | | | | Heptane |
| | | | Acetone | 2-Propanol |
| | | | | Heptane |
| | | | Acetonitrile | 2-Propanol |
| 4-15 kDa | 5% (w/w) | 100 mg/mL | Dichlormethane | 2-Propanol |
| PLGA (75:25), | | | | Heptane |
| Resomer 752H | | | Chloroform | 2-Propanol |
| | | | | Heptane |
| | | | Ethyl Acetate | 2-Propanol |
| | | | | Heptane |
| | | | Tetrahydrofuran | 2-Propanol |
| | | | | Heptane |
| | | | Acetone | 2-Propanol |
| | | | | Heptane |
| | | | Acetonitrile | 2-Propanol |

Although not exemplified, the methods for preparing the polymeric nanoparticles encapsulating peptides can use any suitable solvents and non-solvents, as long as the ΔG_{Mix} of the solvent and non-solvent pairs has a desired value, selected based on the desired size, encapsulation efficiency, and/or release profile. For example, when the desired release profile of the formed nanoparticles is sustained release over a long period of time, such as a lag time period ≥200 hours, without burst release (i.e., ≤20% of theoretical loading at time 0), then the ΔG_{Mix} of the solvent and non-solvent pairs is more negative, such as equals to or more negative than −0.6. When the desired release profile of the formed nanoparticles is rapid release over a short period of time, such as a lag time period ≤24 hours, with burst release (i.e., >20% of theoretical loading at time 0), then the ΔG_{Mix} of the solvent and non-solvent pairs is less negative, such as less negative than-0.6.

As examples, two types of polymer non-solvent were assessed (heptane and 2-propanol). Both the GLP-1 RA and the PLGA are not soluble in the polymer non-solvent. Upon addition of the drug-polymer suspension into the polymer non-solvent, the polymer is precipitated to form the polymeric nanoparticles encapsulating GLP-1 RA. Heptane was used as an exemplary non-polar solvent and 2-propanol was used as an exemplary polar solvent.

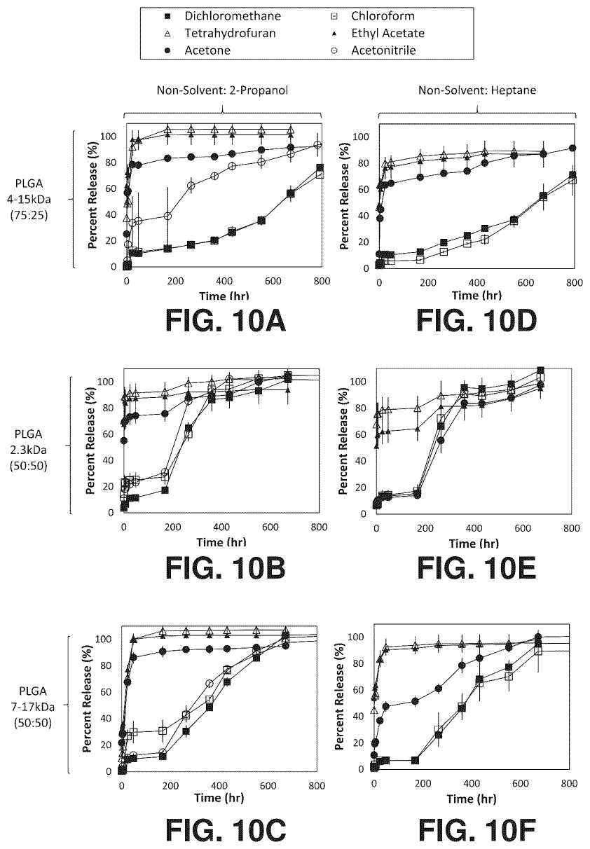

In-vitro drug release experiments were conducted in a PBS medium at pH 7.4 and HPLC was used to quantify the release over time. The in-vitro drug release profiles for each formulation according to Table 5 are shown in FIGS. 10A-10F. FIGS. 10A-10F are organized such that each individual graph displays the various release profiles obtained using different polymer solvents, with the graphs in the left column (i.e., FIGS. 10A-10C) showing the release profiles of particles precipitated into 2-propanol and the graphs in the right column (i.e., FIGS. 10D-10F) showing the release profiles of particles precipitated into heptane. Each row of the graphs represents the particles formed from either PLGA (75:25) 4-15 kDa (i.e., FIGS. 10A and 10D), PLGA (50:50) 2.3 kDa (i.e., FIGS. 10B and 10E), or PLGA (50:50) 7-17 kDa (i.e., FIGS. 10C and 10F).

The impact of solvent and non-solvent on the encapsulation and in-vitro release profile of GLP-1 RA from phase inverted PLGA nanoparticles were determined. Without being bound to any theories, it is believed that the solvent and non-solvent used in phase inversion may have an impact on the affinity for the polymer to encapsulate the peptide. In the PIN process, nanoparticles spontaneously precipitate after the addition of a solubilized polymer solution to a non-solvent. Upon precipitation, the presence of micronized GLP-1 RA particles are expected to act as a core nucleus in which the polymer in solution will precipitate around.

Data described above demonstrate that solvent choice can alter the size and morphology of particle formation by phase inversion. The deviations between solvents are thought to arise from the degree of supersaturation that is achieved based on the ΔG_{Mix} of the solvent and non-solvent pairs. How this mechanism may extend to the PIN process and the ultimate encapsulation and in-vitro release of the GLP-1 RA from PLGA nanoparticles were studied.

When analyzing FIGS. 10A-10F, drug release behavior from PLGA nanoparticles can be divided into four categories: burst release, diffusion release, lag time, and degradation release. The initial burst release refers to the poorly encapsulated drug on the surface of the nanoparticles, which is immediately released upon suspension of the particles. In cases where sustained release is desired, this burst is undesirable as it can result in a loss of drug content or toxic high dosages. Following the initial burst, large macromolecule drugs may diffuse from the polymer particle through a network of pores. While large molecules such as peptides and proteins may not directly diffuse through the polymer matrix, they may release through pores left behind by the surface encapsulated drug from the initial burst. Deeper encapsulated drug is thought to diffuse through the free volume of empty pores left behind by drug released closer to the surface, essentially diffusing though a network of pores. In the absence of this network of pores, large molecule drugs may remain encapsulated by the polymer and result in a lag time. The lag time refers to a period in which no drug release occurs, between the initial burst/diffusion and the onset of polymer degradation. In the case of PLGA, the polymer eventually begins to degrade by hydrolysis. Upon degradation a second phase of drug release occurs, in which the previously encapsulated drug is exposed as the polymer chains break down.

Upon analyzing all 33 GLP-1 RA release profiles in FIGS. 10A-10F, several trends emerged and are discussed in detail below.

### Variations Between Polymer Solvents

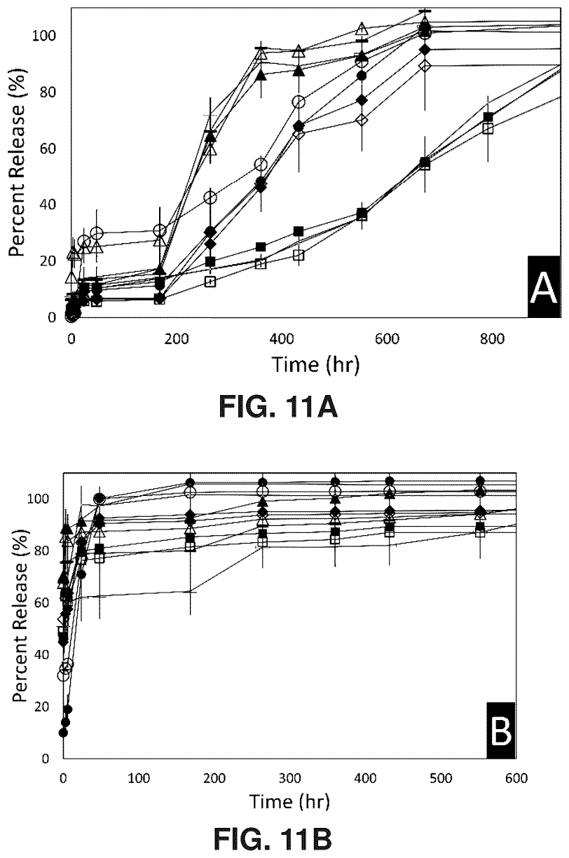

FIG. 11A shows all formulations which utilized dichloromethane or chloroform as a polymer solvent in the PIN process. FIG. 11B shows all those which utilized ethyl acetate or tetrahydrofuran as a polymer solvent. The release profiles of formulations produced with different polymer solvents can be generally broken down into two categories: those which release primarily by degradation (FIG. 11A) and those which release primarily by burst and diffusion (FIG. 11B). As shown in FIG. 11A, across all three PLGA types and varying non-solvent type (2-propanol or heptane), these formulations consistently showed a low burst release (ranging from 0% to 14%), with the majority of drug releasing after a 200-hour lag time and upon the onset of polymer degradation. While on the other hand, as shown in FIG. 11B, formulations which utilized ethyl acetate or tetrahydrofuran showed higher burst release (ranging from 10% to 68%) with a majority of the drug being released at either the 0 hr time point or by diffusion within the first 6-24 hours. In an attempt to understand the mechanism underlying the trend shown in FIGS. 11A and 11B, the release was analyzed as a function of ΔG_{Mix} between the solvents used in PIN and X₁₂ (i.e., the interaction parameter between the polymer and solvent).

### Encapsulation Efficiency and Release Profile as a Function of Solvent/Non-Solvent ΔG_{Mix}

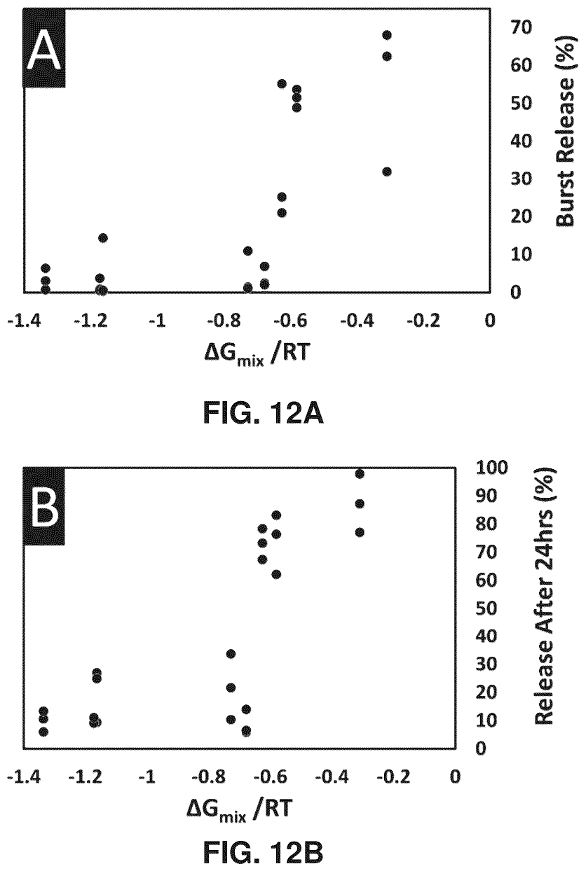

FIGS. 12A and 12B show the relationship between the amount of GLP-1RA released from the PLGA nanoparticles and the ΔG_{Mix} of polymer solvent/poymer non-solvent pair. Specifically, FIG. 12A shows the GLP-1RA burst release at the 0 hr time point as a function of the ΔG_{Mix} between the polymer solvent/polymer non-solvent pair used to produce the nanoparticles. FIG. 12B shows the amount of GLP-1 RA released at the 24 hr time point as a function of the ΔG_{Mix} between the polymer solvent/polymer non-solvent pair used to produce the nanoparticles. Table 6 shows the ΔG_{Mix} of each polymer solvent/polymer non-solvent pair. Acetonitrile and heptane have a positive value of ΔG_{Mix} and are therefore not miscible. For this reason, nanoparticles could not be produce using these solvents and this formulation could not be studied (Table 6). Although acetone and heptane are miscible, they have low favorability of mixing with the least negative ΔG_{Mix}/RT value (Table 6). As a result, nanoparticles formed from these solvents were substantially larger than the other formulations (see FIG. 8). For this reason, the formulations produced from acetone and heptane were also excluded from analysis.

Generally, FIGS. 12A and 12B show that nanoparticles produced from polymer solvents/polymer non-solvent pairs with more negative ΔGₘᵢₓ tend towards a lower GLP-1 RA burst release (FIG. 12A) and lower diffusion within the first 24 hours (FIG. 12B). Table 6 shows that dichloromethane to 2-propanol, dichloromethane to heptane, chloroform to 2-propanol, chloroform to heptane, and acetonitrile to 2-propanol have comparatively low ΔGₘᵢₓ/RT values ranging from −1.336 to −0.678. Correspondingly, FIGS. 12A and 12B show that these formulations have minimal burst release (ranging from 0.33% to 14.4% at time 0) and 24 hour diffusion (ranging from 5.95% to 33.64%). The release curves shown in FIGS. 10A-10F and FIG. 11A show that these formulations retain the GLP-1 RA through a lag period of approximately 200 hours and the majority of release upon the initiation of polymer degradation.

On the other hand, Table 6 shows that ethyl acetate and acetone had comparatively high ΔGₘᵢₓ values with the polymer non-solvent. The nanoparticles formed from these polymer solvent/polymer non-solvent pairs tended towards a larger GLP-1 RA burst release and 24 hour diffusion (FIGS. 12A and 12B). Specifically, Table 6 shows that ethyl acetate to 2-propanol, ethyl acetate to heptane, and acetone to 2-propanol have ΔGₘᵢₓ/RT values ranging from −0.626 to −0.31. Correspondingly, FIGS. 12A and 12B show that nanoparticles formed from the mixing of these polymer solvent/polymer non-solvent pairs release the GLP-1 RA primarily by burst and diffusion, with the burst release ranging from 21% to 68% at time 0 and the 24 hour release ranging from 62.19% to 97.81%.

FIGS. 12A and 12B also show that there is a correlation between the ΔGₘᵢₓ of the polymer solvent/polymer non-solvent and the resulting particle encapsulation efficiency and mode of release. This correlation may be explained by the supersaturation achieved based on the favorability of solvent/non-solvent mixing. Without being bound to any theories, it is believed that increasingly negative ΔGₘᵢₓ between the solvent and non-solvent used to produce the particles indicates a more favorable and thus faster rate of mixing. Increasingly negative ΔGₘᵢₓ between the solvent and non-solvent used to produce peptide nanoparticles and polymeric particles without peptide encapsulation resulted in smaller nanoparticle size. This may relate to the degree of solute supersaturation induced by the mixing, with faster mixing inducing a higher degree of supersaturation and thus more nucleation and smaller growth of particle size. Thus, without being bound to any theories, the GLP-1 RA burst release and mode of release as a function of supersaturation is thought to be achieved by the ΔGₘᵢₓ and the corresponding rate of solvent/non-solvent mixing. Increasingly negative ΔGₘᵢₓ is thought to correspond to an increased rate of mixing between the solvent and non-solvent in the PIN process. Faster rates of mixing are thought to induce a higher state of polymer supersaturation. As the polymer becomes more thermodynamically unstable with increasing supersaturation, the micronized drug in the suspension may become a more favorable center for nucleation and result in better encapsulation.

On the other hand, solvent/non-solvent pairs with less negative ΔGₘᵢₓ are thought to have a slower rate of mixing. Accordingly, slower rates of mixing are expected to induce lowers states of polymer supersaturation. Polymer and drug phase inversion from solvent/non-solvent pairs with less negative ΔGₘᵢₓ resulted in larger particles sizes. The underlying mechanism may be attributed the degree of supersaturation achieved based on the rate of mixing. Less negative ΔGₘᵢₓ results in slower mixing, which in turn results in lower degrees of solute supersaturation. With less supersaturation, the polymer is thought to form fewer nucleation centers and grow larger particles. With less supersaturation the polymer may have a lower affinity for the suspended micronized drug as a nucleation center, resulting in less efficient encapsulation.

TABLE 6
Gibbs energy of mixing between the solvents and
non-solvents used to produce each PIN formulation
ΔGₘᵢₓ /RT

Non-solvents

| Solvents | 2-Propanol | Heptane |
|---|---|---|
| Dichloromethane | −1.173 | −1.336 |
| Chloroform | −1.163 | −0.678 |
| Ethyl Acetate | −0.31 | −0.581 |
| Acetone | −0.626 | −0.219 |
| Acetonitrile | −0.728 | 0.145 |

### Encapsulation Efficiency and Release Profile as a Function of X₁₂

In addition to the ΔGₘᵢₓ between solvent and non-solvent in the PIN process, the PLGA interaction with the solvent and the corresponding impact on encapsulation and release of GLP-1 RA were considered. Specifically, the Flory-Huggins interaction parameter (X₁₂) was used to analyze the affinity between the PLGA and each solvent used in the encapsulation process (Table 7). The interaction parameter X₁₂ is a dimensionless parameter that depends on the nature of both polymer and solvent and defines the total of interactions between pairs of polymer segments, between pairs of solvent molecules, and between polymer segments and solvent molecules. X₁₂ may be determined using the Hildebrand solubility parameter:

$${X}_{12} \approx \frac{{v}_{1}}{RT} {( {δ}_{2} - {δ}_{2} )}^{2}$$

where v₁ is the molar volume of the solvent and δ₁ and δ₂ are the Hildebrand solubility parameters of the solvent and polymer respectively. The criterion of a good solvent is typically regarded as the δ₁≈δ₂. Thus, for this analysis, the general notation is such that a lower X₁₂ value indicates more favorable interaction between polymer and solvent.

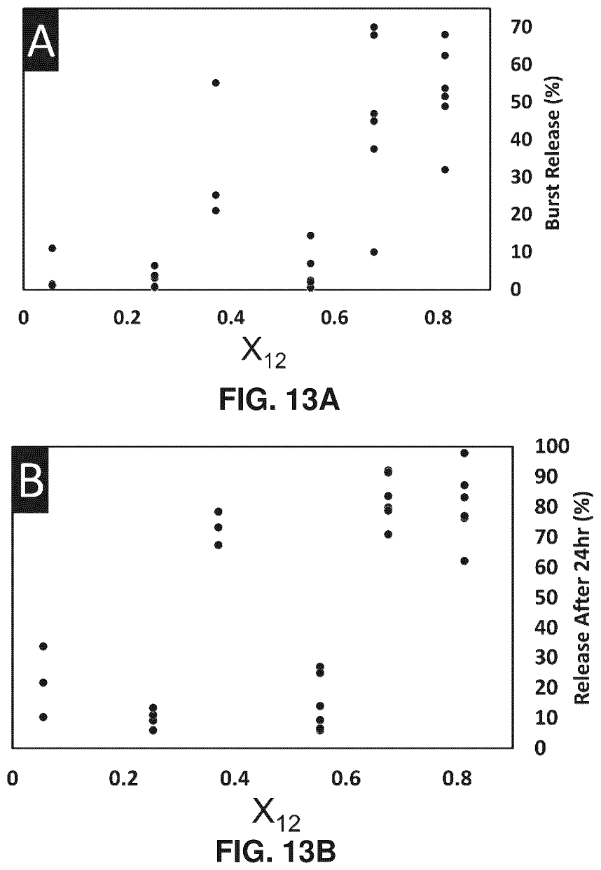

FIGS. 13A and 13B show the relationship between the amount of GLP-1 RA released from the PLGA nanoparticles and the PLGA interaction parameter, X₁₂, with the solvent used to create the particles. Specifically, FIG. 13A shows the GLP-1RA burst release at the 0 hr time point as a function of the X₁₂ between the PLGA and solvent used to produce the nanoparticles. FIG. 13B shows the amount of GLP-1RA released at the 24 hr time point as a function of the X₁₂ between the PLGA and solvent used to produce the nanoparticles. Table 7 shows the X₁₂ of each solvent with PLGA.

Generally, as shown in FIGS. 13A and 13B, nanoparticles produced from solvents with low interaction parameters with the PLGA tend towards a lower GLP-1 RA burst release (FIG. 13A) and lower diffusion within the first 24 hours (FIG. 13B). Table 7 shows that dichloromethane, chloroform, and acetonitrile have comparatively low X₁₂ values with PLGA, ranging from 0.055 to 0.55. Correspondingly, FIGS. 13A and 13B shows these formulations have minimal burst release (ranging from 0.33% to 14.4% at time 0 hr) and 24 hour diffusion (ranging from 5.95% to 33.64%). The release curves from FIGS. 10A-10F and FIG. 11A show that these formulations predominantly retain the GLP-1 RA through a lag period of approximately 200 hours and the majority of release upon the initiation of polymer degradation.

On the other hand, ethyl acetate and tetrahydrofuran show higher X₁₂ values with PLGA (Table 7) and nanoparticles formed from these solvents tended towards a larger GLP-1 RA burst release and 24 hour diffusion (FIGS. 13A and 13B). Table 7 shows that ethyl acetate has a X₁₂ of 0.81 with PLGA, and tetrahydrofuran has a X₁₂ of 0.68 with PLGA. Correspondingly, FIGS. 13A and 13B show that nanoparticles formed from these solvents released the GLP-1 RA primarily by burst and diffusion, with the burst release ranging from 10% to 70% at time 0 hr and the 24 hour release ranging from 62.19% to 97.81%.

FIGS. 13A and 13B show that the interaction parameter between the PLGA and solvent may correspond to the ability of PLGA to encapsulate the micronized GLP-1 RA. This correlation may be explained by the chain conformation of the solubilized PLGA in solvents of varying affinity. Solvents with lower PLGA X₁₂ value are thought to have a higher affinity to the polymer and thus interact with a higher number of monomers within the polymer chain, overall resulting in a more extended chain conformation of the solubilized polymer. On the other hand, solvents with higher PLGA X₁₂ values are thought to have a lower affinity to the polymer and thus interact less favorably with the monomers in the polymer chain, resulting in a more compact chain conformation of the solubilized polymer. The extended PLGA chain conformation facilitated by good solvents such as dichloromethane, chloroform, and acetonitrile may result in a higher state of super saturation and greater ability to encapsulate the micronized GLP-1 RA upon precipitation in the PIN process.

Acetone shows a X₁₂ value of 0.37 with PLGA. Despite having a comparatively low interaction parameter with PLGA (i.e., lower than that of chloroform), acetone releases the GLP-1 RA primarily by burst release (ranging from 21% to 55% at time 0 hr) and 24 hour diffusion (ranging from 67.4% to 78.4%) (FIGS. 13A and 13B). This deviation may arise from the polar nature of acetone. Amongst the six polymer solvents tested in Table 7, acetone and acetonitrile are considered to be polar with dipole moments of 2.88 D and 3.92 D, respectively. Whereas, dichloromethane, chloroform, tetrahydrofuran, and ethyl acetate are considered to be non-polar or only ‘borderline’ polar with dipole moments ranging from 1.04 D to 1.78 D. The encapsulated GLP-1 RA is most soluble in highly polar solvents (i.e., water and methanol). While the suspended micronized GLP-1 RA is not soluble in acetone, it may have a higher degree of solvation with a polar solvent compared to a non-polar solvent. This strong surface interaction of the micronized GLP-1 RA with acetone may in part inhibit its availability to act as a nucleation center upon polymer precipitation in the PIN process. However, this phenomenon was not observed in the nanoparticles produced with acetonitrile (which is also considered highly polar). Acetonitrile has lowest X₁₂ value with PLGA of 0.055 and comparatively lower ΔGₘᵢₓ with the non-solvent when compared to acetone (see Table 6). Thus, a combination of X₁₂, ΔGₘᵢₓ, and polarity may be factors when considering the ability of PLGA to encapsulate the micronized GLP-1 RA.

TABLE 7
Interaction parameters between PLGA and each
solvent used in the encapsulation process

| Polymer | Solvent | X₁₂ |
|---|---|---|
| PLGA | Dichloromethane | 0.25286 |
| | Chloroform | 0.553401 |
| | Tetrahydrofuran | 0.675793 |
| | Ethyl Acetate | 0.812947 |
| | Acetone | 0.370597 |
| | Acetonitrile | 0.055169 |

### Effect of PLGA Monomer Ratio and Molecular Weight on Release

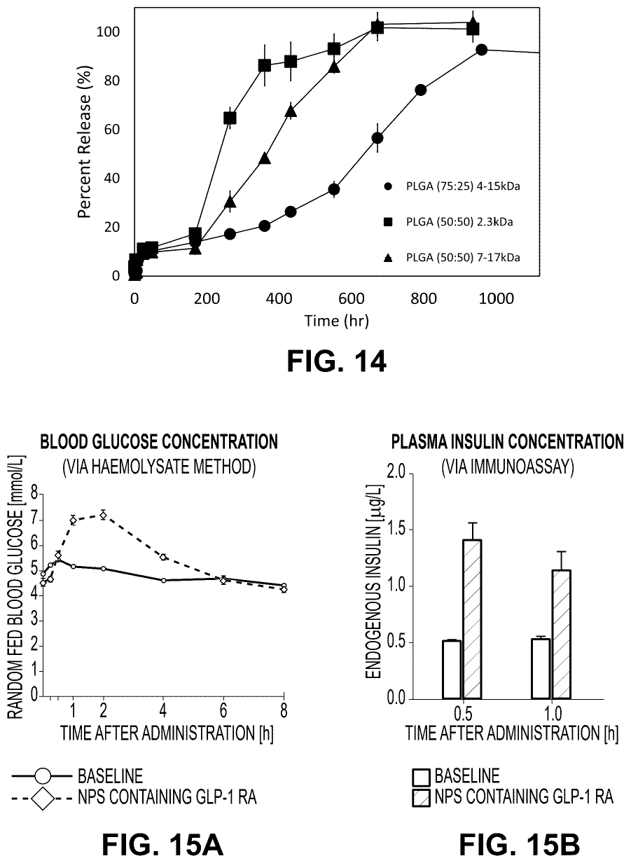

The effect of varying PLGA monomer ratio and molecular weight on the release of GLP-1 RA from PLGA nanoparticles was studied. FIG. 14 shows representative GLP-1 RA release curves from PLGA nanoparticles formed under identical conditions. The only variable between the three release curves in FIG. 14 was the type of PLGA used to encapsulate the peptide (i.e. PLGA (75:25) 4-15 kDa, PLGA (50:50) 2.3 kDa, or PLGA (50:50) 7-17 kDa). FIG. 14 shows all formulations produced with dichloromethane as a polymer solvent and 2-propanol as a polymer non-solvent. Similar trends were observed for formulations produced with dichloromethane/heptane, chloroform/2-propanol, chloroform/heptane, and acetonitrile/2-propanol (FIGS. 10A-10F and 11A). FIG. 14 serves to represent the general trend of varying PLGA type amongst these formulations.

As shown in FIG. 14, all three formulations with varying PLGA type had similar burst release, diffusion, and lag period. With burst release ranging from 0.57% to 3.80% at time 0 hr and release after 24 hours ranging from 9.20% to 11.06%. The lag period appeared to be approximately 200 hours for each formulation. Upon the onset of polymer degradation, each formulation began to release the GLP-1 RA and showed distinct release profile based on the molecular weight and monomer ratio, with the PLGA (50:50) 2.3 kDa releasing at the fastest rate and the PLGA (75:25) 4-15 kDa releasing at the slowest rate.

Generally, PLGA degrades by hydrolysis of ester links resulting in bulk or heterogeneous erosion. Upon addition to an aqueous environment, PLGA begins to hydrate as water penetrates the amorphous regions. Penetration of water molecules disrupts the secondary bonding within the PLGA network, resulting in a decrease in glass transition temperature. This ultimately leads to the initial degradation of the polymer, in which cleavage of covalent ester linkages occurs. Upon the onset of degradation, carboxylic end groups may auto catalyze the degradation process. Finally, the fragments of the degraded polymer chain begin to solubilize and diffuse into the aqueous environment.

The degradation process of PLGA depends on both molecular weight and monomer ratio. Reports have shown that increasing molecular weight from 10-20 to 100 kDa resulted in the degradation varying from several weeks to several months (Gentile, P., Chiono, V., Carmagnola, I. & Hatton, P. Int. J. Mol. Sci. 15, 3640-3659 (2014)). In addition, since lactic acid is more hydrophobic than glycolic acid, PLGA with higher lactic acid content is less hydrophilic, absorbs less water, and degrades more slowly (Gentile, P., Chiono, V., Carmagnola, I. & Hatton, P. Int. J. Mol. Sci. 15, 3640-3659 (2014)). FIG. 14 shows that PLGA (50:50) 2.3 kDa degraded the fastest and released the GLP-1 RA fastest due to the higher glycolic acid content and low molecular weight. Similarly, increasing the molecular weight but keeping the monomer ratio fixed (i.e., PLGA (50:50) 7-17 kDa) resulted in a decreased degradation and release rate. Finally, increasing both the molecular weight and the lactic acid content (i.e., PLGA (75:25) 4-15 kDa) resulted in the slowest degradation and release rate.

### In-Vivo Bioactivity

Nanoparticles containing micronized GLP-1 RA (produced from solvent: water and non-solvent: tert-butanol) were subcutaneously administered to Wistar rats and serial blood samples were then drawn for blood glucose and insulin analysis. In the treatment of type 2 diabetes, GLP-1 RA is required for the production of insulin. In turn, insulin aids in preventing hyperglycemia by transporting glucose from the bloodstream into cells (Donnelly, D. Br. J. Pharmacol. 166, 27-41 (2012)). In rats administered with subcutaneous dosages of PLGA nanoparticles containing micronized GLP-1 RA, the blood glucose and plasma insulin concentrations were increased significantly as compared to baseline values (FIGS. 15A and 15B). In rats there is a paradoxical impact on blood glucose due to activation of the sympathetic nervous system (Bruce, D. G., Chisholm, D. J., Storlien, L. H., Kraegen, E. W. & Smythe, G. A. Diabetologia 35, 835-843 (1992)). This in-vivo glucose and insulin response demonstrate that the micronized GLP-1 RA remains in the native active state.

### CONCLUSION

The data demonstrate successful encapsulation and release of an exemplary GLP-1 RA peptide from PLGA nanoparticles. The PLGA nanoparticles encapsulating GLP-1 RA peptide were produced using a process termed phase inversion nanoencapsulation. Specifically, data was provided towards the mechanism of particle formation by supersaturation, nucleation, and growth upon adding a solubilized polymer or drug solution to a miscible non-solvent. This mechanism is correlated to the Gibbs energy of mixing (ΔG_{Mix}) between the solvent and non-solvent used in the phase inversion process, calculated using the activity coefficients obtained from the UNIFAC method. By choosing the appropriate solvent and non-solvent pairs for the phase inversion process, it is demonstrated that particle size, encapsulation efficiency, and release profile can be modified and tailored for a desired function. For example, the size of GLP-1 RA nanoparticles and PLGA blank nanoparticles tends to decrease with increasingly negative ΔG_{Mix} of the solvent and non-solvent pairs used to produce them. For example, solvent and non-solvent pair with a ΔG_{Mix}/RT value of −1.293 (dichloromethane and heptane) yielded PLGA nanoparticles with a number average size of 260 nm, while those with a less negative ΔG_{Mix}/RT value of −0.205 (acetone and heptane) yielded nanoparticles with a comparatively larger number average size of 638 nm. The ΔG_{Mix} of solvent and non-solvent pairs used in the phase inversion process correlates to the GLP-1 RA encapsulation efficiency and release behavior of the drug from PLGA nanoparticles. GLP-1 RA encapsulated in PLGA nanoparticles using polymer solvent and polymer non-solvent pairs with ΔG_{Mix}/RT values (between approximately −1.4 and −0.6) displayed low burst release (0-20% of the theoretical loading at time 0 hr) and released the majority of drug after an approximate 8-day (200 hour) lag period. On the other hand, GLP-1 RA encapsulated in PLGA nanoparticles using polymer solvent and polymer non-solvent pairs with less negatives ΔG_{Mix}/RT values (between approximately −0.6 and −0.2) displayed large burst release (approximately 20-70% of the theoretical loading at time 0 hr) with the majority of the drug being released by the 1-day (24 hour) time point (approximately 60-100% of the theoretical loading). This correlating between ΔG_{Mix} and particle size, encapsulation efficiency, and release profile may be through the mechanism of supersaturation, nucleation, and growth.

### REFERENCES

1. Brazel, C. & Rosen, S. Fundamental Principles of Polymeric Materials. (Wiley, 2012).
2. Nishida, H. & Tokiwa, Y. Effects of higher-order structure of Poly(3-hydrolxybutyrate) on biodegradation. II Effects of crystal structure on microbial degradation. J. Environ. Polym. Degrad. 1, 65-80 (1993).
3. Shalaev, E., Wu, K., Shamblin, S., Krzyzaniak, J. F. & Descamps, M. Crystalline mesophases: Structure, mobility, and pharmaceutical properties. Adv. Drug Deliv. Rev. 100, 194-211 (2016).
4. Ulery, B. D., Nair, L. S. & Laurencin, C. T. Biomedical applications of biodegradable polymers. J. Polym. Sci. Part B Polym. Phys. 49, 832-864 (2011).
5. Pawlak, A. Cavitation during tensile deformation of high-density polyethylene. Polymer (Guildf). 48, 1397-1409 (2007).
6. Galeski, A. Strength and toughness of crystalline polymer systems. Progress in Polymer Science (Oxford) 28, (2003).
7 Elias, H.-G. Polymerization of Organized Systems. (Gordon & Breach Science Publishers Ltd, 1977).
8. A. Cuculo, John & Chen, G. Extrusion, fiber formation, and characterization of thermotropic copolyesters. J. Polym. Sci. 26, 179-200 (1988).
9. Li, D. & Xia, Y. Electrospinning of Nanofibers: Reinventing the Wheel? Adv. Mater. 16, 1151-1170 (2004).
10. Snyder, J. Notch tip damage zone in biaxially oriented polypropylene at low temperature. Appl. Polym. Sci. 52, 217 (1994).
11. Qiu, J., Murata, T., Wu, X., Kudo, M. & Sakai, E. Plastic deformation mechanism of crystalline polymer materials during the rolling process. J. Mater. Sci. 48, 1920-1931 (2013).
12. Beslikas, T., Ioannis, G., Goulios, V. & Bikiaris, D. Crystallization Study and Comparative in vitro-in vivo hydrolysis of PLA reinforcement ligament. Int. J. Mol. Sci. 12, (2011).
13. LAWRENCE, M. J. Biodegradable Polymers as Drug Delivery Systems. (Drugs in the Pharmaceutical Sciences Series/45): Edited by Mark Chasin and Robert Langer. J. Pharm. Pharmacol. 44, 72-72 (1992).
14. Thornton, A. W., Nairn, K. M., Hill, A. J. & Hill, J. M. New relation between diffusion and free volume: I. Predicting gas diffusion. 338, 29-37 (2009).
15. Crisóstomo, J. et al. Increasing levels of insulin secretion in bioartificial pancreas technology: co-encapsulation of beta cells and nanoparticles containing GLP-1 in alginate hydrogels. Health Technol. (Berl). 10, 885-890 (2020).
16. Uhl, P. file:///C:/Users/cbaptist/Documents/Solubilit. P. 1039@c6nr00294. G.-1 P. double emulsion. pd. et al. Coating of PLA-nanoparticles with cyclic, arginine-rich cell penetrating peptides enables oral delivery of liraglutide. Nanomedicine Nanotechnology, Biol. Med. 24, 102132 (2020).
17. Tomoda, K., Yabuki, N., Terada, H. & Makino, K. Surfactant free preparation of PLGA nanoparticles: The combination of antisolvent diffusion with preferential solvation. Colloids Surfaces A Physicochem. Eng. Asp. 457, 88-93 (2014).
18. Zhang, L. et al. Tf ligand-receptor-mediated exenatide-Zn2+ complex oral-delivery system for penetration enhancement of exenatide. Journal of Drug Targeting 26, (Taylor & Francis, 2018).
19. Zhang, L. et al. The use of low molecular weight protamine to enhance oral absorption of exenatide. International Journal of Pharmaceutics 547, (2018).
20. Araújo, F. et al. In vivo dual-delivery of glucagon like peptide-1 (GLP-1) and dipeptidyl peptidase-4 (DPP4) inhibitor through composites prepared by microfluidics for diabetes therapy. Nanoscale 8, 10706-10713 (2016).
21. Dong, N. et al. Development of composite PLGA microspheres containing exenatide-encapsulated lecithin nanoparticles for sustained drug release. Asian J. Pharm. Sci. 15, 347-355 (2020).
22. Eissa, N. G., Elsabahy, M. & Allam, A. Engineering of smart nanoconstructs for delivery of glucagon-like peptide-1 analogs. Int. J. Pharm. 597, 120317 (2021).
23. Ismail, R. et al. Encapsulation in polymeric nanoparticles enhances the enzymatic stability and the permeability of the glp-1 analog, liraglutide, across a culture model of intestinal permeability. Pharmaceutics 11, 1-13 (2019).
24. Kankala, R. K. et al. Supercritical Fluid-Assisted Decoration of Nanoparticles on Porous Microcontainers for Codelivery of Therapeutics and Inhalation Therapy of Diabetes. ACS Biomater. Sci. Eng. 4, 4225-4235 (2018).
25. Senduran, N. et al. Orally deliverable nanoformulation of liraglutide against type 2 diabetic rat model. J. Drug Deliv. Sci. Technol. 56, 101513 (2020).
26. Shi, Y. et al. Fc-modified exenatide-loaded nanoparticles for oral delivery to improve hypoglycemic effects in mice. Sci. Rep. 8, 1-9 (2018).
27. Song, Y. et al. Synthesis of CSK-DEX-PLGA Nanoparticles for the Oral Delivery of Exenatide to Improve Its Mucus Penetration and Intestinal Absorption. Mol. Pharm. 16, 518-532 (2019).
28. Yu, M. et al. Battle of GLP-1 delivery technologies. Adv. Drug Deliv. Rev. 130, 113-130 (2018).
29. Li, T. et al. Characterization of attributes and in vitro performance of exenatide-loaded PLGA long-acting release microspheres. Eur. J. Pharm. Biopharm. 158, 401-409 (2021).
30. Lewis, A. Formulation strategies for sustained release of proteins. Ther. Deliv. 457-479 (2010).
31. Wang, J., Wang, B. M. & Schwendeman, S. P. Characterization of the initial burst release of a model peptide from poly(D,L-lactide-co-glycolide) microspheres. J. Control. Release 82, 289-307 (2002).
32. Tracy, M. A. et al. Factors affecting the degradation rate of poly(lactide-co-glycolide) microspheres in vivo and in vitro. Biomaterials 20, 1057-1062 (1999).
33. Mathiowitz, E. Encyclopedia of Controlled Drug Delivery. (Wiley, 1999).
34. Alexis, F., Pridgen, E., Molnar, L. K. & Farokhzad, O. C. Factors affecting the clearance and biodistribution of polymeric nanoparticles. Mol. Pharm. 5, 505-515 (2008).
35. Siepmann, J., Faisant, N., Akiki, J., Richard, J. & Benoit, J. P. Effect of the size of biodegradable microparticles on drug release: experiment and theory. 96, 123-134 (2004).
36. Tsoi, K. M. et al. Mechanism of hard nanomaterial clearance by the liver. Nat. Mater. 15, 1212-1221 (2016).
37. Wang, H. et al. Improved Oral Bioavailability and Liver Targeting of Sorafenib Solid Lipid Nanoparticles in Rats. AAPS PharmSciTech (2017). doi: 10.1208/s12249-017-0901-3
38. Mathiowitz, E. Oral delivery of proteins by biodegradable nanoparticles. Adv. Drug Deliv. Rev. 65, 811-821 (2013).
39. Klose, D., Siepmann, F., Elkharraz, K. & Siepmann, J. PLGA-based drug delivery systems: Importance of the type of drug and device geometry. 354, 95-103 (2008).
40. Operti, M. C. et al. PLGA-based nanomedicines manufacturing: Technologies overview and challenges in industrial scale-up. Int. J. Pharm. 605, 120807 (2021).
41. Richard, J. Challenges in the delivery of peptide drugs: an industry perspective. 6, 149-163 (2015).
42. Pace, C. N. et al. Protein structure, stability and solubility in water and other solvents. 42. Philos. Trans. R. Soc. B Biol. Sci. 359, 1225-1235 (2004).
43. Cicerone, M. T., Pikal, M. J. & Qian, K. K. Stabilization of proteins in solid form. Adv. Drug Deliv. Rev. 93, 14-24 (2015).
44. Aggarwal, V. & Richardson, J. Improving enzymes by using them in organic solvents. Heteroat. Analog. Aldehydes Ketones 409, 1 (2004).
45. Jacob, J. S. & Mathiowitz, E. A Novel Mechanism for Spontaneous Encapsulation of Active Agents: Phase Inversion Nanoencapsulation. Carr. Based Drug Deliv. 214-223 (2004). doi: 10.1021/bk-2004-0879.ch015
46. Martínez Rivas, C. J. et al. Nanoprecipitation process: From encapsulation to drug delivery. Int. J. Pharm. 532, 66-81 (2017).
47. Shibaev, V. & Lam, L. Liquid Crystalline and Mesomorphic Polymers. (1994).
48. Wunderlich, B. A classification of molecules, phases, and transitions as recognized by thermal analysis. Thermochim. Acta 340, 37-52 (1999).
49. Hanna, S., Windle, A. & Donald, A. Liquid Crystalline Polymers. (Cambridge University Press, 2006).
50. Peters, S. T. Handbook of Composites. (Springer-Verlag, 1998).
51. Gordon, M. Liquid Crystal Polymers II/III. (Springer, 1984).
52. Hirte, R. No Title. Acta Polym. 36, 242-242 (1985).
53. Gennes, P. G. de. Physics of Liquid Crystals. (Oxford Press, 1974).
54. Baker, C., Azagury, A. & Mathiowitz, E. Effect of pressure on poly-L-Lactic Acid morphology. Polymer (Guildf). 99, 250-262 (2016).
55. PerkinElmer. Differential Scanning calorimetry: A Beginner's Guide. 1-9 (2014).
56. Cassel, B. & Packer, R. Modulated Temperature DSC: A Step Up in Performanc. PerkinElmer
57. Post, B. X-ray diffraction methods in polymer science, Leroy E. Alexander, wiley-interscience, new york, 1970. xv+582 pp. J. Polym. Sci. Part B Polym. Lett. 9, 635-636 (1971).
58. Weber, M. F. Giant Birefringent Optics in Multilayer Polymer Mirrors. Science (80-.). 287, 2451-2456 (2000).
59. Lorenz, M., Evers, A. & Wagner, M. Recent progress and future options in the development of GLP-1 receptor agonists for the treatment of diabesity. Bioorganic Med. Chem. Lett. 23, 4011-4018 (2013).
60. Inzucchi, S. E. et al. Management of Hyperglycemia in Type 2 Diabetes: A Patient-Centered Approach. Diabetes Care 35, 1364-1379 (2012).
61. Collins L, C. R. Glucagon-like Peptide-1 Receptor Agonists. StatPearls Publishing (2021). Available at: https://www.ncbi.nlm.nih.gov/books/NBK551568/.
62. Anderson, J. M. & Shive, M. S. Biodegradation and biocompatibility of PLA and PLGA microspheres. 28, 5-24 (1997).
63. Hoffman, A. S. The origins and evolution of “controlled” drug delivery systems. J. Control. Release 132, 153-163 (2008).
64. Wischke, C. & Schwendeman, S. P. Principles of encapsulating hydrophobic drugs in PLA/PLGA microparticles. 364, 298-327 (2008).
65. Carino, G. P., Jacob, J. S. & Mathiowitz, E. Nanosphere based oral insulin delivery. J. Control. Release 65, 261-269 (2000).
66. Azagury, A. et al. Single Step Double-walled Nanoencapsulation (SSDN). J. Control. Release 280, 11-19 (2018).
67. Sovan Lal Pal, Utpal Jana, P. K. Manna, G. P. Mohanta, R. M. Nanoparticle: An overview of preparation and characterization. J. Appl. Pharm. Sci. 6, 228-234 (2011).
68. Stetefeld, J., McKenna, S. A. & Patel, T. R. Dynamic light scattering: a practical guide and applications in biomedical sciences. Biophys. Rev. 8, 409-427 (2016).
So, P. & Broutman, L. J. Residual stresses in polymers and their effect on mechanical 69. behavior. Polym. Eng. Sci. 16, 785-791 (1976).
70. ZOETELIEF, W. F., DOUVEN, L. F. A. & INGEN HOUSZ, A. J. Residual Thermal Stresses in Injection Molded Products. Polym. Eng. Sci. 36, 1866-1896 (1996).
71. Schreiber, T., Schultz, H. & Schmidt, O. Stress-induced birefringence in large-mode-area micro-structured optical fibers. Opt. Express 13, (2005).
72. Hammack, T. J. & Andrews, R. D. Temperature Dependence of Orientation Birefringence of Polymers in the Glassy and Rubbery States. II. J. Appl. Phys. 36, 3574-3581 (1965).
73. Gent, A. Stress-induced birefringence of swollen polymer networks. Macromolecules 2, 262-265 (1969).
74. La Mantia, F. P., Ceraulo, M., Mistretta, M. C. & Morreale, M. Effect of cold drawing on mechanical properties of biodegradable fibers. J. Appl. Biomater. Funct. Mater. 0-0 (2016). doi: 10.5301/jabfm.5000328
75. Stoclet, G., Seguela, R., Lefebvre, J. M., Elkoun, S. & Vanmansart, C. Strain-induced molecular ordering in polylactide upon uniaxial stretching. Macromolecules 43, 1488-1498 (2010).
76. Lan, Q., Li, Y. & Chi, H. Highly Enhanced Mesophase Formation in Glassy Poly(l-lactide) at Low Temperatures by Low-Pressure CO2That Provides Moderately Increased Molecular Mobility. Macromolecules 49, 2262-2271 (2016).
77. Liang, Y. Y., Tang, H., Zhong, G. J. & Li, Z. M. Formation of poly(L-lactide) mesophase and its chain mobility dependent kinetics. Chinese J. Polym. Sci. (English Ed. 32, 1176-1187 (2014).
78. Stoclet, G., Seguela, R., Lefebvre, J. M. & Rochas, C. New insights on the strain-induced mesophase of poly(d, 1-lactide): In situ WAXS and DSC study of the thermo-mechanical stability. Macromolecules 43, 7228-7237 (2010).
79. Lv, R. et al. Mesophase formation and its thermal transition in the stretched glassy polylactide revealed by infrared spectroscopy. Polymer (Guildf). 52, 4979-4984 (2011).
80. Zhou, C., Li, H., Zhang, W., Li, J. & Huang, S. Direct investigations on strain-induced cold crystallization behavior and structure evolutions in amorphous poly(lactic acid) with SAXS and WAXS measurements. Polymer (Guildf). 90, 111-121 (2016).
81. Androsch, R., Laura, M., Lorenzo, D., Schick, C. & Wunderlich, B. Mesophases in polyethylene, polypropylene, and poly (1-butene). Polymer (Guildf). 51, 4639-4662 (2010).
82. Garlotta, D. A Literature Review of Poly (Lactic Acid). 9, (2002).
83. Woodruff, M. A. & Hutmacher, D. W. The return of a forgotten polymer-Polycaprolactone in the 21st century. Prog. Polym. Sci. 35, 1217-1256 (2010).
84. Langer, R. & Chasin, M. Biodegradable polymers as drug delivery systemsitle. (1990).
85. Dash, T. K. & Konkimalla, V. B. Poly-&-caprolactone based formulations for drug delivery and tissue engineering: A review. J. Control. Release 158, 15-33 (2012).
86. Jame, M. Polymer Data Handbook. (2009).
87. Labet, M. & Thielemans, W. Synthesis of polycaprolactone: a review. Chem. Soc. Rev. 38, 3484 (2009).
88. Chandra, R. Biodegradable polymers. Prog. Polym. Sci. 23, 1273-1335 (1998).
89. Middleton, J. C. & Tipton, J. Synthetic biodegradable polymers as orthopedic devices. Biomaterials 21, (2000).
90. Woodward, S. C., Brewer, P. S., Moatamed, F., Schindler, A. & Pitt, C. G. The intracellular degradation of poly(?-caprolactone). J. Biomed. Mater. Res. 19, 437-444 (1985).
91. McEachin, Z. & Lozano, K. Production and characterization of polycaprolactone nanofibers via Forcespinning™ technology. J. Appl. Polym. Sci. 126, 473-479 (2012).
92. Hermanová, S. et al. The Effect of Processing of Polycaprolactone Films on Degradation Process Initiated by Aspergillus Oryzae Lipase. Int. J. Polym. Anal. Charact. 17, 465-475 (2012).
93. Agarwal, S. & Speyerer, C. Degradable blends of semi-crystalline and amorphous branched poly(caprolactone): Effect of microstructure on blend properties. Polymer (Guildf). 51, 1024-1032 (2010).
94. Wang, Y., Rodriguez-Perez, M. A., Reis, R. L. & Mano, J. F. Thermal and Thermomechanical Behaviour of Polycaprolactone and Starch/Polycaprolactone Blends for Biomedical Applications. Macromol. Mater. Eng. 290, 792-801 (2005).
95. McKeen, L. Renewable Resource and Biodegradable Polymers. in The Effect of Sterilization on Plastics and Elastomers 305-317 (Elsevier, 2012). doi: 10.1016/B978-1-4557-2598-4.00012-5
96. Hu, H. & Dorset, D. L. Crystal structure of poly(iε-caprolactone). Macromolecules 23, 4604-4607 (1990).
97. Allegra, G. Interphases and Mesophases in Polymer Crystallization II. 181, (Springer Berlin Heidelberg, 2005).
98. De Rosa, C. et al. Crystalline Ethylene-Norbornene Copolymers: Plastic Crystals from Macromolecules. Macromolecules 36, 3789-3792 (2003).
99. Keith, H. D. & Passaglia, E. Dislocations in Polymer Crystals. J. Res. Natl. Bur. Stand. (1934). 68A, 513-518 (1964).
100. Tani, Y. C. H. A., Okita, Y., Adokoro, H. T. & Amashita, Y. Structural Studies of Polyesters. III. Crystal Structure of Poly-e-caprolactone. 1, 555-562 (1970).
101. Bassett, D. C., Block, S. & Piermarini, G. J. A high-pressure phase of polyethylene and chain-extended growth. J. Appl. Phys. 45, 4146-4150 (1974).
102. Wunderlich, B. & Arakawa, T. Polyethylene crystallized from the melt under elevated pressure. J. Polym. Sci. Part A Gen. Pap. 2, 3697-3706 (1964).
103. Cheng, J. et al. Identification and quantitative analysis of the intermediate phase in a linear high-density polyethylene. J. Polym. Sci. Part B Polym. Phys. 32, 2683-2693 (1994).
104. Chatani, Y., Okita, Y., Tadokoro, H. & Yamashita, Y. Structural Studies of Polyesters. III. Crystal Structure of Poly-8-caprolactone. Polym. J. 1, 555-562 (1970).
105. Yamamoto, T., Miyaji, H. & Asai, K. Structure and Properties of High Pressure Phase of Polyethylene. Jpn. J. Appl. Phys. 16, 1891-1898 (1977).
106. Wunderlich, B., Möller, M., Grebowicz, J. & Baur, H. Conformational Motion and Disorder in Low and High Molecular Mass Crystals. 87, (Springer Berlin Heidelberg, 1988).
107. Zhuravlev, E., Schmelzer, J. W. P., Wunderlich, B. & Schick, C. Kinetics of nucleation and crystallization in poly(¿-caprolactone) (PCL). Polymer (Guildf). 52, 1983-1997 (2011).
108. Liu, X., Zhao, Y., Fan, X. & Chen, E. Crystal orientation and melting behavior of poly(ε-Caprolactone) under one-dimensionally “hard” confined microenvironment. Chinese J. Polym. Sci. 31, 946-958 (2013).
109. Wang, Y. et al. Unusual structural evolution of poly(lactic acid) upon annealing in the presence of an initially oriented mesophase. Soft Matter 10, 1512-1518 (2014).
110. Wang, Y. et al. Spectroscopic analysis of post drawing relaxation in poly(lactic acid) with oriented mesophase. Polym. Test. 43, 103-107 (2015).
111. Puchalski, M., Kwolek, S., Szparaga, G., Chrzanowski, M. & Krucińska, I. Investigation of the Influence of PLA Molecular Structure on the Crystalline Forms (α′ and α) and Mechanical Properties of Wet Spinning Fibres. Polymers (Basel). 9, 18 (2017).
112. Grassia, L., Pastore Carbone, M. G., Mensitieri, G. & D′Amore, A. Modeling of density evolution of PLA under ultra-high pressure/temperature histories. Polymer (Guildf). 52, 4011-4020 (2011).
113. Baptista, C. et al. The effect of temperature and pressure on polycaprolactone morphology. Polymer (Guildf). 191, 122227 (2020).
114. Wasanasuk, K. et al. Crystal Structure Analysis of Poly(1-lactic Acid) a Form On the basis of the 2-Dimensional Wide-Angle Synchrotron X-ray and Neutron Diffraction Measurements. Macromolecules 44, 6441-6452 (2011).
115. John, S. Structural Studies of Liquid Crystals by X-ray Diffraction. in Handbook of Liquid Crystals 635-655 (1998). doi: 10.1002/9783527671403
116. Mulligan, J. & Cakmak, M. Nonlinear Mechanooptical Behavior of Uniaxially Stretched Poly (lactic acid): Dynamic Phase Behavior. 2333-2344 (2005). doi: 10.1021/ma048794f
117. Monnier, X. et al. Molecular dynamics in electrospun amorphous plasticized polylactide fibers. Polymer (Guildf). 73, 68-78 (2015).
118. Na, B. et al. Unusual Cold Crystallization Behavior in Physically Aged Poly(l-lactide). J. Phys. Chem. B 115, 10844-10848 (2011).
119. Park, J. B. & Uhlmann, D. R. Recovery of deformed polymers. III. Thermal properties. J. Appl. Phys. 44, 201-206 (1973).
120. Hasan, O. & Boyce, M. Energy storage during inelastic deformation of glassy polymers. Polymer (Guildf). 34, 5085-5092 (1993).
121. Pawlak, A. & Gałeski, A. Cavitation during tensile drawing of semicrystalline polymers. Polimery/Polymers 56, 627-636 (2011).
122. Galeski, A. Strength and toughness of crystalline polymer systems. Prog. Polym. Sci. 28, 1643-1699 (2003).
123. Z.-Q.HuA.-M.WangH.-F.Zhang. Modern Inorganic Synthetic Chemistry (Second Edition). (2017). doi: https://doi.org/10.1016/B978-0-444-63591-4.00022-7
124. Spathis, G. & Kontou, E. Nonlinear viscoelastic and viscoplastic response of glassy polymers. Polym. Eng. Sci. 41, 1337-1344 (2001).
125. Hasan, O. A. & Boyce, M. C. A constitutive model for the nonlinear viscoelastic viscoplastic behavior of glassy polymers. Polym. Eng. Sci. 35, 331-344 (1995).
126. Di Vona, M. L. Annealing of Polymer Membranes. in Encyclopedia of Membranes 1-2 (Springer Berlin Heidelberg, 2014). doi: 10.1007/978-3-642-40872-4_627-1
127. Parker, M. J. & Warton, B. A. E. S. Test Methods for Physical Properties.
128. Chung, Y. G., Lacks, D. J. & Chung, Y. G. Sheared polymer glass and the question of mechanical rejuvenation Sheared polymer glass and the question of mechanical rejuvenation. 124907, (2012).
129. Kulinski, Z. & Piorkowska, E. Crystallization, structure and properties of plasticized poly(l-lactide). Polymer (Guildf). 46, 10290-10300 (2005).
130. Solarski, S., Ferreira, M. & Devaux, E. Characterization of the thermal properties of PLA fibers by modulated differential scanning calorimetry. Polymer (Guildf). 46, 11187-11192 (2005).
131. Pluta, M. & Galeski, A. Crystalline and supermolecular structure of polylactide in relation to the crystallization method. J. Appl. Polym. Sci. 86, 1386-1395 (2002).
132. Kim, M. S., Kim, J. C. & Kim, Y. H. Effects of take-up speed on the structure and properties of melt-spun poly(L-lactic acid) fibers. Polym. Adv. Technol. 19, 748-755 (2008).
133. Pan, P., Zhu, B. & Inoue, Y. Enthalpy Relaxation and Embrittlement of Poly(<scp>1</scp>-lactide) during Physical Aging. Macromolecules 40, 9664-9671 (2007).
134. Lee, S. C., Han, J. Il, Jeong, Y. G. & Kwon, M. Strain-Induced Enthalpy Relaxation in Poly (lactic acid). Macromolecules 43, 25-28 (2010).
135. Na, B. et al. Spectroscopic Evidence of Melting of Ordered Structures in the Aged Glassy Poly(l-lactide). Macromolecules 43, 1702-1705 (2010).
136. Kovarova, M. et al. Ultra-long-acting removable drug delivery system for HIV treatment and prevention. Nat. Commun. 9, 4156 (2018).
137. Abdelkader, H. et al. Polymeric long-acting drug delivery systems (LADDS) for treatment of chronic diseases: Inserts, patches, wafers, and implants. Adv. Drug Deliv. Rev. 177, 113957 (2021).
138. Siepmann, J. & Peppas, N. A. Higuchi equation: Derivation, applications, use and misuse. Int. J. Pharm. 418, 6-12 (2011).
139. PEPPAS, N. A. & LUSTIG, S. R. The Role of Cross-links, Entanglements, and Relaxations of the Macromolecular Carrier in the Diffusional Release of Biologically Active Materials. Ann. N. Y. Acad. Sci. 446, 26-40 (1985).
140. Baptista, C., Azagury, A., Baker, C. M. & Mathiowitz, E. The characterization and quantification of the induced mesophases of poly-1-lactic acid. Polymer (Guildf). 226, 123822 (2021).
141. Higuchi, T. Rate of Release of Medicaments from Ointment Bases Containing Drugs in Suspension. J. Pharm. Sci. 50, 874-875 (1961).
142. W.H.O. Diabetes (Fact sheet no. 312). World Health Organization Available at: https://www.who.int/en/news-room/fact-sheets/detail/diabetes. (Accessed: 15 Oct. 2021)
143. I.D.F. IDF Diabetes Atlast Update, 5th ed. International Diabetes Federation (2012). Available at: http://www.idf.org/diabetesatlas. (Accessed: 15th October 2021)
144. Evers, A. et al. Dual Glucagon-like Peptide 1 (GLP-1)/Glucagon Receptor Agonists Specifically Optimized for Multidose Formulations. J. Med. Chem. 61, 5580-5593 (2018).
145. Prausnitz, J., Lichtenthaler, R. & Gomes de Azevedo, E. Molecular Thermodynamics of Fluid-Phase Equilibria. (Prentice Hall PTR, 1999).
146. Fredenslund, A., Gmehling, J. & Rasmussen, P. Vapor-liquid Equilibria Using Unifac. (Elsevier, 1977). doi: 10.1016/B978-0-444-41621-6.X5001-7
147. Lira, C. & Elliott, R. Introductory Chemical Engineering Thermodynamics. (Pearson, 2012).
148. Lira, C. & Elliott, R. Activity Coefficient Excel File. Michigan State University (2021). Available at: https://www.egr.msu.edu/˜lira/computer/EXCEL/ACTCOEFF.XLS. (Accessed: 5th October 2021)
149. Daniel, J. & Renardy, Y. Fundamentals of Two-Fluid Dynamics. (Springer, 1993).
150. Donnelly, D. The structure and function of the glucagon-like peptide-1 receptor and its ligands. Br. J. Pharmacol. 166, 27-41 (2012).
151. Nelson, D. & Cox, M. Lehninger Principles of Biochemistry. (Macmillian Learning, 2021).
152. Lapanje, S. Physicochemical Aspects of Protein Denaturation. (Wiley, 1978).
153. Fitzpatrick, P. A., Steinmetz, A. C., Ringe, D. & Klibanov, A. M. Enzyme crystal structure in a neat organic solvent. Proc. Natl. Acad. Sci. 90, 8653-8657 (1993).
154. Yennawar, N. H., Yennawar, H. P. & Farber, G. K. X-ray Crystal Structure of .gamma.-Chymotrypsin in Hexane. Biochemistry 33, 7326-7336 (1994).
155. Schmitke, J. L., Stern, L. J. & Klibanov, A. M. The crystal structure of subtilisin Carlsberg in anhydrous dioxane and its comparison with those in water and acetonitrile. Proc. Natl. Acad. Sci. 94, 4250-4255 (1997).
156. Zhu, G. et al. X-ray studies on two forms of bovine β-trypsin crystals in neat cyclohexane. Biochim. Biophys. Acta-Protein Struct. Mol. Enzymol. 1429, 142-150 (1998).
157. Gao, X.-G. et al. Crystal structure of triosephosphate isomerase from Trypanosoma cruzi in hexane. Proc. Natl. Acad. Sci. 96, 10062-10067 (1999).
158. Klibanov, A. Enzymatic catalysis in anhydrous organic solvents. Trends Biochem. Sci. 14, 141-144 (1989).
159. Adlercreutz, P. Modes of using enzymes in organic media. in Enzymatic Reactions in Organic Media 9-42 (Springer Netherlands, 1996). doi: 10.1007/978-94-011-0611-5_2
160. Miller, L. M., Bourassa, M. W. & Smith, R. J. FTIR spectroscopic imaging of protein aggregation in living cells. Biochim. Biophys. Acta-Biomembr. 1828, 2339-2346 (2013).
161. Jackson, M. & Mantsch, H. H. The Use and Misuse of FTIR Spectroscopy in the Determination of Protein Structure. Crit. Rev. Biochem. Mol. Biol. 30, 95-120 (1995).
162. Bruce, D. G., Chisholm, D. J., Storlien, L. H., Kraegen, E. W. & Smythe, G. A. The effects of sympathetic nervous system activation and psychological stress on glucose metabolism and blood pressure in subjects with Type 2 (non-insulin-dependent) diabetes mellitus. Diabetologia 35, 835-843 (1992).
163. Minkin, V. I., Osipov, O. A. & Zhdanov, Y. A. Dipole Moments in Organic Chemistry. (Springer US, 1970). doi: 10.1007/978-1-4684-1770-8
164. Gentile, P., Chiono, V., Carmagnola, I. & Hatton, P. An Overview of Poly (lactic-co-glycolic) Acid (PLGA)-Based Biomaterials for Bone Tissue Engineering. Int. J. Mol. Sci. 15, 3640-3659 (2014).
165. Houchin, M. L. & Topp, E. M. Physical properties of PLGA films during polymer degradation. J. Appl. Polym. Sci. 114, 2848-2854 (2009).

1. A pharmaceutical composition for delivering a peptide comprising a micronized peptide encapsulated or dispersed in a particle,
wherein the particle comprises one or more polymers,
wherein the peptide has a molecular weight of up to 6,000 Da,
wherein the particle has a number average size of 10 microns or less, and
wherein the composition provides sustained release of the peptide with less than 20% of the peptide released initially (0 hour) following placement into a phosphate buffered saline at 37° C. and room pressure (i.e., 1 atm).

2. (canceled)

3. The pharmaceutical composition of claim 1, wherein less than 50% of the peptide is released at 24 hours.

4. The pharmaceutical composition of claim 1, wherein less than 50% of the peptide is released at 200 hours.

5. The pharmaceutical composition of claim 1, wherein ≥50% of the peptide is released at or after 400 hours.

6. The pharmaceutical composition of claim 1, wherein the particle has a number average size of 5 microns or less, 2 microns or less, 750 nm or less, 500 nm or less, or 300 nm or less.

7. The pharmaceutical composition of claim 1, wherein the polymer is a biodegradable polymer, such as a polymer selected from the group consisting of biodegradable polyesters (e.g., polyhydroxyesters), polyanhydrides, poly(lactic acid), poly(glycolic acid), and poly(lactic-co-glycolic acid), or a copolymer thereof, or a mixture thereof.

8. The pharmaceutical composition of claim 1, wherein the polymer is PLGA.

9. The pharmaceutical composition of claim 8, wherein the PLGA has a molecular weight in a range from about 2 kDa to about 20 kDa, from about 2 kDa to about 3 kDa, from about 4 kDa to about 15 kDa, or from about 7 kDa to about 17 kDa.

10. The pharmaceutical composition of claim 8, wherein the weight ratio of lactic acid to glycolic acid in the PLGA is in a range from 25:75 to 75:25, such as 50:50 or 75:25.

11. The pharmaceutical composition of claim 1, wherein the micronized peptide is a glucagon-like peptide-1 receptor agonist (“GLP-1 RA”).

12. A method for micronizing a peptide, comprising:
(i) dissolving the peptide in an effective amount of a peptide solvent to form a peptide solution,
(ii) introducing the peptide solution into a peptide non-solvent,
wherein the peptide solvent and the peptide non-solvent are miscible, and
wherein the Gibbs energy of mixing (ΔGₘᵢₓ/RT) for the peptide solvent and the peptide non-solvent is less than or equal to about −0.6.

13. The method of claim 12, wherein during step (ii), the peptide is precipitated to produce a composition comprising micronized nanoparticles of the peptide, and wherein the micronized nanoparticles have a number average size of 5 microns or less, 1 micron or less, 300 nm or less.

14. The method of claim 12, wherein the peptide solvent is methanol or water, or a combination thereof, and/or wherein the peptide non-solvent is selected from the group consisting of tert-butanol, 2-propanol, acetonitrile, acetone, ethyl acetate, tetrahydrofuran, dichloromethane, and chloroform, or a combination thereof.

15. The method of claim 12, wherein the peptide is a glucagon-like peptide-1 receptor agonist (“GLP-1 RA”).

16. A method for forming particles comprising a solid, micronized peptide, comprising:
(a) dissolving a polymer in a first suspension to form a second suspension, wherein the first suspension comprises the solid micronized peptide and a polymer solvent, and
(b) introducing the second suspension into a polymer non-solvent to spontaneously form the particles,
wherein the polymer solvent and the polymer non-solvent are miscible, and
wherein the Gibbs energy of mixing (ΔGₘᵢₓ/RT) for the polymer solvent and the polymer non-solvent is less than or equal to about −0.6.

17. The method of claim 16, wherein the method further comprises, prior to step (a),
(i) micronizing a peptide to form the first suspension comprising the micronized peptide.

18. The method of claim 16, wherein step (b) does not include emulsification, agitation, and/or stirring.

19. The method of claim 16, wherein the polymer solvent is dichloromethane or chloroform, or a combination thereof, and/or wherein the polymer non-solvent is 2-propanol or heptane, or a combination thereof.

20.-30. (canceled)

31. The pharmaceutical composition of claim 1, wherein the micronized peptide has a number average size of 5 microns or less, 1 micron or less, 300 nm or less.

32. The pharmaceutical composition of claim 1, wherein the micronized peptide is glucagon-like peptide-1 or a glucagon-like peptide-1 analogue.


SEQUENCE LISTING (10 sequences)

SEQ ID NO: 1 - protein, 37 aa, Homo sapiens
HDEFERHAEGTFTSDVSSYLEGQAAKEFIAWLVKGRG

SEQ ID NO: 2 - protein, 31 aa, Homo sapiens
HAEGTFTSDVSSYLEGQAAKEFIAWLVKGRG

SEQ ID NO: 3 - protein, 30 aa, Homo sapiens
HAEGTFTSDVSSYLEGQAAKEFIAWLVKGR

SEQ ID NO: 4 - protein, 39 aa, synthetic construct
HGEGTFTSDLSKQMEEEAVRLFIEWLKNGGPSSGAPPPS

SEQ ID NO: 5 - protein, 31 aa, synthetic construct
HAEGTFTSDVSSYLEGQAAKEFIAWLVRGRG

SEQ ID NO: 6 - protein, 44 aa, synthetic construct
HGEGTFTSDLSKQMEEEAVRLFIEWLKNGGPSSGAPPSKKKKKK

SEQ ID NO: 7 - protein, 645 aa, synthetic construct
HGEGTFTSDVSSYLEGQAAKEFIAWLVKGRHGEGTFTSDVSSYLEGQAAKEFIAWLVKGRDAHKSEVAHRFKDLGEENFKALVLIAFAQYLQQCPFEDHVKLVNEVTEFAKTCVADESAENCDKSLHTLFGDKLCTVATLRETYGEMADCCAKQEPERNECFLQHKDDNPNLPRLVRPEVDVMCTAFHDNEETFLKKYLYEIARRHPYFYAPELLFFAKRYKAAFTECCQAADKAACLLPKLDELRDEGKASSAKQRLKCASLQKFGERAFKAWAVARLSQRFPKAEFAEVSKLVTDLTKVHTECCHGDLLECADDRADLAKYICENQDSISSKLKECCEKPLLEKSHCIAEVENDEMPADLPSLAADFVESKDVCKNYAEAKDVFLGMFLYEYARRHPDYSVVLLLRLAKTYETTLEKCCAAADPHECYAKVFDEFKPLVEEPQNLIKQNCELFEQLGEYKFQNALLVRYTKKVPQVSTPTLVEVSRNLGKVGSKCCKHPEAKRMPCAEDYLSVVLNQLCVLHEKTPVSDRVTKCCTESLVNRRPCFSALEVDETYVPKEFNAETFTFHADICTLSEKERQIKKQTALVELVKHKPKATKEQLKAVMDDFAAFVEKCCKADDKETCFAEEGKKLVAASQAALGL

SEQ ID NO: 8 - protein, 275 aa, synthetic construct
HGEGTFTSDVSSYLEEQAAKEFIAWLVKGGGGGGGSGGGGSGGGGSAESKYGPPCPPCPAPEAAGGPSVFLFPPKPKDTLMISRTPEVTCVVVDVSQEDPEVQFNWYVDGVEVHNAKTKPREEQFNSTYRVVSVLTVLHQDWLNGKEYKCKVSNKGLPSSIEKTISKAKGQPREPQVYTLPPSQEEMTKNQVSLTCLVKGFYPSDIAVEWESNGQPENNYKTTPPVLDSDGSFFLYSRLTVDKSRWQEGNVFSCSVMHEALHNHYTQKSLSLSLG

SEQ ID NO: 9 - protein, 31 aa, synthetic construct
  MOD_RES 2: note = X = alpha-aminoisobutyric acid
  SITE 20: note = Lys20 is acylated with C-18 stearic diacid
HXEGTFTSDVSSYLEGQAAKEFIAWLVRGRG

SEQ ID NO: 10 - protein, 30 aa, synthetic construct
  MOD_RES 29: note = x= 2-methylalanine
  MOD_RES 2: note = X = 2-methylalanine
  MOD_RES 30: note = X is L-argininamide
HXEGTFTSDVSSYLEGQAAKEFIAWLVKXX